(19) United States
(12) Patent Application Publication
(10) Pub. No.: US 2024/0158510 A1
(43) Pub. Date: May 16, 2024

(54) ANTIBODIES AGAINST INTEGRIN HETERODIMERS AND USES THEREOF

(71) Applicant: The Governing Council of the University of Toronto, Toronto (CA)

(72) Inventors: Sachdev S. SIDHU, Toronto (CA); Eugenio GALLO, Toronto (CA); Jarrett J. ADAMS, Toronto (CA); Levi Lynn BLAZER, Toronto (CA); Rodilia CARDARELLI, Toronto (CA); Abdellali KELIL, Toronto (CA)

(73) Assignee: The Governing Council of the University of Toronto, Toronto, ON (CA)

(21) Appl. No.: 18/549,837

(22) PCT Filed: Mar. 8, 2022
(86) PCT No.: PCT/IB2022/052070

§ 371 Date: Sep. 8, 2023

Related U.S. Application Data
(60) Provisional application No. 63/158,769, filed on Mar. 9, 2021.

(51) Int. Cl. C07K 16/28; A61P 35/00

(52) U.S. Cl. CPC C07K 16/2842; A61P 35/00; C07K 16/2818; C07K 2317/31; C07K 2317/76

(57) ABSTRACT

The disclosure provides antibodies that bind human ITGAv/B1 and methods of use thereof. In some aspects, the disclosure is directed to methods of treating a cancer in a subject, comprising administering to the subject an anti-ITGAv/B1 antibody.

### CROSS-REFERENCE TO RELATED APPLICATIONS

This application claims priority benefit of U.S. Provisional Application No. 63/158,769, filed Mar. 9, 2021, which is herein incorporated by reference in its entirety.

### REFERENCE TO SEQUENCE LISTING SUBMITTED ELECTRONICALLY VIA EFS-WEB

The content of the electronically submitted sequence listing (Name: 4756_001PC01_Seqlisting_ST25.txt; Size: 68,219 Bytes; and Date of Creation: Mar. 7, 2022) submitted in this application is incorporated herein by reference in its entirety.

### BACKGROUND OF THE DISCLOSURE

The αv family of RGD-binding integrins have been identified as regulators of transforming growth factor β1 (TGFβ1), a pleiotropic cytokine that can function as a tumour promoter. αv family integrins are able to activate TGFβ1 in vivo and modulate tumour progression indirectly via the local production of active TGFβ1. TGFβ1 is a powerful activator of the trans-differentiation of fibroblasts into myofibroblasts. Thus, αv family integrins activate TGFβ1 and induce myofibroblast formation resulting in myofibroblast-dependent activities that include matrix-remodelling, matrix-stiffening and cancer promotion. Here, TGFβ1 induces fibroblasts to adopt a contractile wound-repair phenotype leading to trans-differentiation in the tumour microenvironment that result in cancer-associated fibroblasts (CAFs) or tumour-associated fibroblasts (TAFs). Moreover, TGFβ1 can induce some non-fibroblast cells, including adipocytes and circulating bone-marrow-derived suppressor cells, to trans-differentiate into cancer-associated myofibroblasts.

By increasing the contraction of the extracellular matrix, myofibroblasts also increase the likelihood of activating latent TGFβ1. For instance, myofibroblasts secrete a huge number of proteins that further enhance cancer progression that include ECM proteases, growth factors, cytokines and chemokines. Similarly, TGFβ1-activated fibroblasts can secrete osteopontin, an RGD-containing integrin ligand implicated in the promotion of tumour growth, EMT and metastasis. Thus, increased osteopontin correlates with increased metastasis and often poor survival in multiple types of cancer including laryngeal squamous cell carcinoma, melanoma, nasopharyngeal carcinoma and breast cancer.

TGFβ1 further promotes angiogenesis, where local activation of TGFβ by αv integrins promote the development of blood vessels in tumors. In addition, different αv integrins are also upregulated on endothelial cells of new blood vessels that promote their migration, with integrins αvβ3, αvβ5, and αvβ8 all regulating angiogenesis.

Activation of TGFβ by any αv integrin can influence local inflammatory and immune cells, promoting immunosuppressive effects on various effector T-cells and inducing tumour-promoting phenotypes in both neutrophils and macrophages. Here, TGFβ1 promotes the formation of tumour-promoting M2 tumour-associated macrophages and N2 tumour-associated neutrophils. Macrophages present as having “M1” show anti-tumour properties; however, “M2” display tumour-promoting characteristics that increase the transcription of TGFβ1, TGFBRI and TGFBRII in carcinoma cells.

TGFβ1 also stimulates monocyte recruitment and alters the inflammatory gene expression profile of macrophages by increasing metastasis-associated interleukin-6 (IL-6) and suppressing cytokines such as IL-10 and chemoattractants CCL3 and CCL4. TGFβ1 stimulation of macrophages also promotes angiogenesis under hypoxic conditions by the elevated production of VEGF, MMP-9 and VEGF receptor Flk-1 expression. Moreover, high levels of M2 macrophages also correlate with poor survival from different cancers. These include pancreatic and cervical cancer, gastric cancer spread and relapse after chemotherapy. Furthermore, multiple studies also suggest M2 cells promote metastasis.

Blockade of TGFβ receptors with inhibitory antibodies would promote whole body targeting with detrimental side effects. However, suitable targeted therapies that minimise off-target effects, and result in local control of TGFβ activation have not yet been developed. Described herein are therapeutic antibodies that specifically target αv integrins as a means of regulating TGFβ1 activity in cancer and fibrosis.

### SUMMARY OF THE DISCLOSURE

Some aspects of the present disclosure are directed to a bispecific antibody or an antigen-binding portion thereof that specifically binds an integrin-αv heterodimer and inhibits integrin-αv-mediated activation of TGFβ.

In some aspects, the bispecific antibody or antigen-binding portion comprises at least a first paratope and a second paratope, wherein the first paratope binds a first epitope on an integrin αvβ1 heterodimer. In some aspects, the second paratope binds a second epitope on the integrin αvβ1 heterodimer. In some aspects, the first epitope and the second epitope are not the same.

In some aspects, the bispecific antibody or antigen-binding portion thereof comprises a first heavy chain, a first light chain, a second heavy chain, and second light chain. In some aspects, the first heavy chain and the second heavy chain are different. In some aspects, the first heavy chain and the second heavy chain are different, and wherein the first light chain and the second light chain are the same.

In some aspects, the first heavy chain comprises a first variable heavy region (“VH1”), comprising a variable heavy complementarity determining region (VH1-CDR) 1, a VH1-CDR2, and a VH1-CDR3; wherein the VH1-CDR3 comprises an amino acid sequence selected from SEQ ID NOs: 5, 15, and 25. In some aspects, the VH1-CDR2 comprises an amino acid sequence selected from SEQ ID NOs: 4, 14, and 24. In some aspects, the VH1-CDR1 comprises an amino acid sequence selected from SEQ ID NOs: 3, 13, and 23. In some aspects, the first light chain comprises a first variable light region (“VL1”), comprising a VL1-CDR1, a VL1-CDR2, and a VL1-CDR3; wherein the VL1-CDR3 comprises an amino acid sequence selected from SEQ ID NOs: 10, 20, and 30. In some aspects, the VL1-CDR2 comprises an amino acid sequence selected from SEQ ID NOs: 9, 19, and 29. In some aspects, the VL1-CDR1 comprises an amino acid sequence selected from SEQ ID NOs: 8, 18, and 28.

In some aspects, the bispecific antibody or antigen-binding portion thereof of any one of claims 1 to 13, comprises: (i) a VH1-CDR1 comprising the amino acid sequence set forth in SEQ ID NO: 3; (ii) a VH1-CDR2 comprising the amino acid sequence set forth in SEQ ID NO: 4; (iii) a VH1-CDR3 comprising the amino acid sequence set forth in SEQ ID NO: 5; (iv) a VL1-CDR1 comprising the amino acid sequence set forth in SEQ ID NO: 18; (v) a VL1-CDR2 comprising the amino acid sequence set forth in SEQ ID NO: 19; and (vi) a VL1-CDR3 comprising the amino acid sequence set forth in SEQ ID NO: 20.

In some aspects, the bispecific antibody or antigen-binding portion thereof comprises: (i) a VH1-CDR1 comprising the amino acid sequence set forth in SEQ ID NO: 13; (ii) a VH1-CDR2 comprising the amino acid sequence set forth in SEQ ID NO: 14; (iii) a VH1-CDR3 comprising the amino acid sequence set forth in SEQ ID NO: 15; (iv) a VL1-CDR1 comprising the amino acid sequence set forth in SEQ ID NO: 18; (v) a VL1-CDR2 comprising the amino acid sequence set forth in SEQ ID NO: 19; and (vi) a VL1-CDR3 comprising the amino acid sequence set forth in SEQ ID NO: 20.

In some aspects, the bispecific antibody or antigen-binding portion thereof comprises: (i) a VH1-CDR1 comprising the amino acid sequence set forth in SEQ ID NO: 3; (ii) a VH1-CDR2 comprising the amino acid sequence set forth in SEQ ID NO: 4; (iii) a VH1-CDR3 comprising the amino acid sequence set forth in SEQ ID NO: 5; (iv) a VL1-CDR1 comprising the amino acid sequence set forth in SEQ ID NO: 28; (v) a VL1-CDR2 comprising the amino acid sequence set forth in SEQ ID NO: 29; and (vi) a VL1-CDR3 comprising the amino acid sequence set forth in SEQ ID NO: 30.

In some aspects, the bispecific antibody or antigen-binding portion thereof comprises: (i) a VH1-CDR1 comprising the amino acid sequence set forth in SEQ ID NO: 23; (ii) a VH1-CDR2 comprising the amino acid sequence set forth in SEQ ID NO: 24; (iii) a VH1-CDR3 comprising the amino acid sequence set forth in SEQ ID NO: 25; (iv) a VL1-CDR1 comprising the amino acid sequence set forth in SEQ ID NO: 28; (v) a VL1-CDR2 comprising the amino acid sequence set forth in SEQ ID NO: 29; and (vi) a VL1-CDR3 comprising the amino acid sequence set forth in SEQ ID NO: 30. In some aspects, the second heavy chain comprises a second variable heavy region (“VH2”), comprising a VH2-CDR1, a VH2-CDR2, and a VH2-CDR3; wherein the VH2-CDR3 comprises an amino acid sequence selected from SEQ ID NOs: 5, 15, and 25. In some aspects, the VH2-CDR2 comprises an amino acid sequence selected from SEQ ID NOs: 4, 14, and 24. In some aspects, the VH2-CDR1 comprises an amino acid sequence selected from SEQ ID NOs: 3, 13, and 23. In some aspects, the second light chain comprises a second variable light region (“VL1”), comprising a VL2-CDR1, a VL2-CDR2, and a VL2-CDR3; wherein the VL2-CDR3 comprises an amino acid sequence selected from SEQ ID NOs: 20 and 30. In some aspects, the VL2-CDR2 comprises an amino acid sequence selected from SEQ ID NOs: 19 and 29. In some aspects, the VH2-CDR1 comprises an amino acid sequence selected from SEQ ID NOs: 18 and 28.

In some aspects, the bispecific antibody or antigen-binding portion thereof comprises: (a) a first variable heavy region (VH1), comprising (i) a VH1-CDR1 comprising the amino acid sequence set forth in SEQ ID NO: 3, (ii) a VH1-CDR2 comprising the amino acid sequence set forth in SEQ ID NO: 4, and (iii) a VH1-CDR3 comprising the amino acid sequence set forth in SEQ ID NO: 5; (b) a first variable light region (VL1), comprising (i) a VL1-CDR1 comprising the amino acid sequence set forth in SEQ ID NO: 18, (ii) a VL1-CDR2 comprising the amino acid sequence set forth in SEQ ID NO: 19, and (iii) a VL1-CDR3 comprising the amino acid sequence set forth in SEQ ID NO: 20; (c) a second variable heavy region (VH2), comprising (i) a VH2-CDR1 comprising the amino acid sequence set forth in SEQ ID NO: 13; (ii) a VH2-CDR2 comprising the amino acid sequence set forth in SEQ ID NO: 14; (iii) a VH2-CDR3 comprising the amino acid sequence set forth in SEQ ID NO: 15; and (d) a second variable light region (VL2), comprising (i) a VL2-CDR1 comprising the amino acid sequence set forth in SEQ ID NO: 18; (ii) a VL2-CDR2 comprising the amino acid sequence set forth in SEQ ID NO: 19; and (iii) a VL2-CDR3 comprising the amino acid sequence set forth in SEQ ID NO: 20.

In some aspects, the bispecific antibody or antigen-binding portion thereof comprises: (a) a first variable heavy region (VH1), comprising (i) a VH1-CDR1 comprising the amino acid sequence set forth in SEQ ID NO: 3, (ii) a VH1-CDR2 comprising the amino acid sequence set forth in SEQ ID NO: 4, and (iii) a VH1-CDR3 comprising the amino acid sequence set forth in SEQ ID NO: 5; (b) a first variable light region (VL1), comprising (i) a VL1-CDR1 comprising the amino acid sequence set forth in SEQ ID NO: 28, (ii) a VL1-CDR2 comprising the amino acid sequence set forth in SEQ ID NO: 29, and (iii) a VL1-CDR3 comprising the amino acid sequence set forth in SEQ ID NO: 30; (c) a second variable heavy region (VH2), comprising (i) a VH2-CDR1 comprising the amino acid sequence set forth in SEQ ID NO: 23; (ii) a VH2-CDR2 comprising the amino acid sequence set forth in SEQ ID NO: 24; (iii) a VH2-CDR3 comprising the amino acid sequence set forth in SEQ ID NO: 25; and (d) a second variable light region (VL2), comprising (i) a VL2-CDR1 comprising the amino acid sequence set forth in SEQ ID NO: 28; (ii) a VL2-CDR2 comprising the amino acid sequence set forth in SEQ ID NO: 29; and (iii) a VL2-CDR3 comprising the amino acid sequence set forth in SEQ ID NO: 30.

In some aspects, the bispecific antibody or antigen-binding portion thereof comprises a first variable heavy region (VH1) and a first variable light region (VL1), wherein the VH1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22.

In some aspects, the VL1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

In some aspects, the bispecific antibody or antigen-binding portion thereof comprises a second variable heavy region (VH2) and a second variable light region (VL2), wherein the VH2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22.

In some aspects, the VL2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

In some aspects, (a) the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; (b) the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17; (c) the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12; and (d) the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17.

In some aspects, (a) the VH1 comprises the amino acid sequence set forth in SEQ ID NO: 2; (b) the VL1 comprises the amino acid sequence set forth in SEQ ID NO: 17; (c) the VH2 comprises the amino acid sequence set forth in SEQ ID NO: 12; and (d) the VL2 comprises the amino acid sequence set forth in SEQ ID NO: 17.

In some aspects, (a) the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; (b) the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27; (c) the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 22; and (d) the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

In some aspects, (a) the VH1 comprises the amino acid sequence set forth in SEQ ID NO: 2; (b) the VL1 comprises the amino acid sequence set forth in SEQ ID NO: 27; (c) the VH2 comprises the amino acid sequence set forth in SEQ ID NO: 22; and (d) the VL2 comprises the amino acid sequence set forth in SEQ ID NO: 27.

In some aspects, bispecific antibody or antigen-binding portion thereof comprises a first heavy chain (H1) and a first light chain (L1), wherein the H1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37.

In some aspects, the L1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

In some aspects, the bispecific antibody or antigen-binding portion thereof comprises a second heavy chain (H2) and a second light chain (L2), wherein the H2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37.

In some aspects, the L2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

In some aspects, the first heavy chain is associated with the second heavy chain. In some aspects, the first heavy chain is associated with the second heavy chain by a covalent bond. In some aspects, each of the first heavy chain and the second heavy chain comprises an IgG constant region or an IgG constant region comprising one or more amino acid substitutions. In some aspects, the one or more amino acid substitutions promotes heterodimerization of the first heavy chain and the second heavy chain.

In some aspects, the first heavy chain comprises a substitution of one or more amino acids in a constant region of the first heavy chain, creating a knob; wherein the second heavy chain comprises a substitution or antigen-binding portion thereof of one or more amino acids in a constant region of the second heavy chain, creating a hole; wherein the knob of the first heavy chain associates with the hole of the second heavy chain.

In some aspects, the first heavy chain comprises a substitution of one or more amino acids in a constant region of the first heavy chain, creating a hole; wherein the second heavy chain comprises a substitution of one or more amino acids in a constant region of the second heavy chain, creating a knob; wherein the hole of the first heavy chain associates with the knob of the second heavy chain.

In some aspects, (a) the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31; (b) the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16; (c) the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 34; and (d) the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, (a) the H1 comprises the amino acid sequence set forth in SEQ ID NO: 31; (b) the L1 comprises the amino acid sequence set forth in SEQ ID NO: 16; (c) the H2 comprises the amino acid sequence set forth in SEQ ID NO: 34; and (d) the L2 comprises the amino acid sequence set forth in SEQ ID NO: 16.

In some aspects, (a) the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31; (b) the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26; (c) the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 37; and (d) the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26. In some aspects, (a) the H1 comprises the amino acid sequence set forth in SEQ ID NO: 31; (b) the L1 comprises the amino acid sequence set forth in SEQ ID NO: 26; (c) the H2 comprises the amino acid sequence set forth in SEQ ID NO: 37; and (d) the L2 comprises the amino acid sequence set forth in SEQ ID NO: 26.

In some aspects, the bispecific antibody has one or more properties selected from the group consisting of: (a) the bispecific antibody inhibits binding of integrin-αvβ1 to LAP-TGFβ1; (b) the bispecific antibody is capable of binding an integrin αv selected from αvβ1, αvβ3, αvβ5, αvβ6, αvβ8, and any combination thereof, (c) the bispecific antibody inhibits cell adhesion; (d) the bispecific antibody inhibits tumor growth and/or metastasis; (e) the bispecific antibody increases progression-free survival; (f) the bispecific antibody increases overall survival; and (g) any combination thereof. In some aspects, the bispecific antibody or antigen-binding portion thereof is capable of (i) inhibiting the binding of integrin-αvβ1 to LAP-TGFβ1 and (ii) inhibiting cell adhesion. In some aspects, the bispecific antibody or antigen-binding portion thereof binds integrin-αv with a K_{D} of 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 25. In some aspects, the VH-CDR2 comprises an amino acid sequence of SEQ ID NO: 4, 14, or 24. In some aspects, the VH-CDR1 comprises an amino acid sequence of SEQ ID NO: 3, 13, or 23. In some aspects, the VL-CDR3 comprises an amino acid sequence SEQ ID NO: 10, 20, or 30. In some aspects, the VL-CDR2 comprises an amino acid sequence of SEQ ID NO: 9, 19, or 29. In some aspects, the VL-CDR1 comprises an amino acid sequence of SEQ ID NOs: 8, 18, or 28.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein (i) the VH-CDR1 comprises the amino acid sequence set forth in SEQ ID NO: 3; (ii) the VH-CDR2 comprises the amino acid sequence set forth in SEQ ID NO: 4; (iii) the VH-CDR3 comprises the amino acid sequence set forth in SEQ ID NO: 5; (iv) the VL-CDR1 comprises the amino acid sequence set forth in SEQ ID NO: 8; (v) the a VL-CDR2 comprises the amino acid sequence set forth in SEQ ID NO: 9; and (vi) the VL-CDR3 comprises the amino acid sequence set forth in SEQ ID NO: 10.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein (i) the VH-CDR1 comprises the amino acid sequence set forth in SEQ ID NO: 13; (ii) the VH-CDR2 comprises the amino acid sequence set forth in SEQ ID NO: 14; (iii) the VH-CDR3 comprises the amino acid sequence set forth in SEQ ID NO: 15; (iv) the VL-CDR1 comprises the amino acid sequence set forth in SEQ ID NO: 18; (v) the VL-CDR2 comprises the amino acid sequence set forth in SEQ ID NO: 19; and (vi) the a VL-CDR3 comprises the amino acid sequence set forth in SEQ ID NO: 20.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein (i) the VH-CDR1 comprises the amino acid sequence set forth in SEQ ID NO: 23; (ii) the VH-CDR2 comprises the amino acid sequence set forth in SEQ ID NO: 24; (iii) the VH-CDR3 comprises the amino acid sequence set forth in SEQ ID NO: 25; (iv) the VL-CDR1 comprises the amino acid sequence set forth in SEQ ID NO: 28; (v) the VL-CDR2 comprises the amino acid sequence set forth in SEQ ID NO: 29; and (vi) the VL-CDR3 comprises the amino acid sequence set forth in SEQ ID NO: 30.

In some aspects, the antibody or antigen binding portion thereof comprises a heavy chain variable region (“VH”) and a light chain variable region (“VL”); wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 2, 12, and 22.

In some aspects, the antibody or antigen binding portion thereof comprises a heavy chain variable region (“VH”) and a light chain variable region (“VL”); wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, and 27.

In some aspects, (a) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7; (b) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 12; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17; or (c) the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 22; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

In some aspects, (a) the VH comprises the amino acid sequence set forth in SEQ ID NO: 2, and wherein the VL comprises the amino acid sequence set forth in SEQ ID NO: 7; (b) the VH comprises the amino acid sequence set forth in SEQ ID NO: 12, and wherein the VL comprises the amino acid sequence set forth in SEQ ID NO: 17; or (c) the VH comprises the amino acid sequence set forth in SEQ ID NO: 22, and wherein the VL comprises the amino acid sequence set forth in SEQ ID NO: 27.

In some aspects, the antibody or antigen binding portion thereof comprises a heavy chain (“HC”) and a light chain (“LC”); wherein the HC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 11, and 21.

In some aspects, the LC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 6, 16, and 16. In some aspects, the antibody or antigen binding portion thereof comprises a heavy chain (“HC”) and a light chain (“LC”); wherein the HC comprises an amino acid sequence selected from SEQ ID NOs: 1, 11, and 21. In some aspects, the LC comprises an amino acid sequence selected from SEQ ID NOs: 6, 16, and 26.

In some aspects, the antibody or antigen binding portion thereof comprises a heavy chain (“HC”) and a light chain (“LC”); wherein: (a) the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 1; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 6; (b) the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16; or (c) the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 21; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26.

In some aspects, the antibody or antigen binding portion thereof comprises a heavy chain (“HC”) and a light chain (“LC”); wherein the HC comprises the amino acid sequence set forth in SEQ ID NO: 1; and the LC comprises the amino acid sequence set forth in SEQ ID NO: 6. In some aspects, the antibody or antigen binding portion thereof comprises a heavy chain (“HC”) and a light chain (“LC”); wherein the HC comprises the amino acid sequence set forth in SEQ ID NO: 11; and the LC comprises the amino acid sequence set forth in SEQ ID NO: 16. In some aspects, the antibody or antigen binding portion thereof comprises a heavy chain (“HC”) and a light chain (“LC”); wherein the HC comprises the amino acid sequence set forth in SEQ ID NO: 21; and the LC comprises the amino acid sequence set forth in SEQ ID NO: 26.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to an amino acid sequence of SEQ ID NO: 2, 12, or 22. In some aspects, the VL comprises an amino acid sequence having at least about 80%, at least about 85%, at least about 90%, at least about 95%, at least about 96%, at least about 97%, at least about 98%, or at least about 99% sequence identity to an amino acid sequence of SEQ ID NO: 7, 17, or 27.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 22; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 2; and the VL comprises the amino acid sequence set forth in SEQ ID NO: 7.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 12; and the VL comprises the amino acid sequence set forth in SEQ ID NO: 17.

Some aspects of the present disclosure are directed to an isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 22; and the VL comprises the amino acid sequence set forth in SEQ ID NO: 27.

In some aspects, the antibody or antigen-binding portion thereof is an antigen-binding portion. In some aspects, the antigen-binding portion is a Fab, Fab′, F(ab′)2, single chain Fv (scFv), disulfide linked Fv, IgNar, intrabody, IgGΔCH2, minibody, F(ab′)3, tetrabody, triabody, diabody, single-domain antibody, DVD-Ig, Fcab, mAb2, (scFv)2, or scFv-Fc.

Some aspects of the present disclosure are directed to a bispecific antibody comprising an antibody or antigen-binding portion thereof disclosed herein, wherein the bispecific antibody is capable of inhibiting integrin-αv-mediated activation of TGFβ.

Some aspects of the present disclosure are directed to a multispecific antibody comprising a bispecific antibody disclosed herein or an antibody or antigen-binding portion thereof disclosed herein, wherein the multispecific antibody is capable of inhibiting integrin-αv-mediated activation of TGFβ.

Some aspects of the present disclosure are directed to an antibody drug conjugate comprising a bispecific antibody or antigen-binding portion thereof disclosed herein, an antibody or antigen-binding portion thereof disclosed herein, or a multispecific antibody disclosed herein. In some aspects, the bispecific antibody or antigen-binding portion thereof, the antibody or antigen-binding portion thereof, or the multispecific antibody is fused to a detectable marker.

Some aspects of the present disclosure are directed to a polynucleotide or a set of polynucleotides encoding a bispecific antibody disclosed herein, an antibody or antigen-binding portion thereof disclosed herein, or a multispecific antibody disclosed herein.

Some aspects of the present disclosure are directed to a set of polynucleotides comprising: (i) a first polynucleotide encoding a first heavy chain disclosed herein, (ii) a second polynucleotide encoding a second heavy chain disclosed herein, and (iii) a third polynucleotide encoding a first light chain disclosed herein.

Some aspects of the present disclosure are directed to a vector or a set of vectors comprising a polynucleotide of a set of polynucleotides disclosed herein.

Some aspects of the present disclosure are directed to a cell comprising a bispecific antibody disclosed herein, an antibody or antigen-binding portion thereof of any one disclosed herein, a multispecific antibody disclosed herein, or a polynucleotide of a set of polynucleotides disclosed herein.

Some aspects of the present disclosure are directed to a pharmaceutical composition comprising a bispecific antibody disclosed herein, an antibody or antigen-binding portion thereof disclosed herein, a multispecific antibody disclosed herein, or a polynucleotide of a set of polynucleotides disclosed herein, and a pharmaceutically acceptable excipient.

Some aspects of the present disclosure are directed to a method of making a bispecific antibody or an antigen-binding portion thereof, an antibody or an antigen-binding portion thereof, or a multispecific antibody or an antigen-binding portion thereof, comprising culturing a host cell disclosed herein under suitable conditions and isolating the bispecific antibody.

Some aspects of the present disclosure are directed to a method of treating a cancer in a subject in need thereof, comprising administering to the subject a bispecific antibody or antigen-binding portion thereof disclosed herein, an antibody or antigen-binding portion thereof disclosed herein, a multispecific antibody or antigen-binding portion thereof disclosed herein, an antibody drug conjugate disclosed herein, a polynucleotide of a set of polynucleotides disclosed herein, a vector or set of vectors disclosed herein, a cell disclosed herein, or a pharmaceutical composition disclosed herein. In some aspects, administration of the bispecific antibody reduces or inhibits metastasis of the cancer in the subject.

Some aspects of the present disclosure are directed to a method of reducing or inhibiting cancer metastasis in a subject in need thereof, comprising administering to the subject a bispecific antibody or antigen-binding portion thereof disclosed herein, an antibody or antigen-binding portion thereof disclosed herein, a multispecific antibody or antigen-binding portion thereof disclosed herein, an antibody drug conjugate disclosed herein, a polynucleotide of a set of polynucleotides disclosed herein, a vector or set of vectors disclosed herein, a cell disclosed herein, or a pharmaceutical composition disclosed herein.

Some aspects of the present disclosure are directed to a method of inhibiting integrin-αvβ1-mediated activation of TGFβ in a subject in need thereof, comprising administering to the subject a bispecific antibody or antigen-binding portion thereof disclosed herein, an antibody or antigen-binding portion thereof of disclosed herein, a multispecific antibody or antigen-binding portion thereof disclosed herein, an antibody drug conjugate disclosed herein, a polynucleotide of a set of polynucleotides disclosed herein, a set of polynucleotides disclosed herein, a vector or set of vectors disclosed herein, a cell disclosed herein, or a pharmaceutical composition disclosed herein.

In some aspects, the subject is afflicted with a cancer. In some aspects, the cancer comprises a tumor. In some aspects, the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

In some aspects, the method further comprises administering to the subject an additional anti-cancer therapy. In some aspects, the additional anti-cancer therapy comprises a chemotherapy, an immunotherapy, a surgery, a radiotherapy, or any combination thereof. In some aspects, the additional anti-cancer therapy comprises a standard of care therapy.

In some aspects, the additional anti-cancer therapy comprises a checkpoint inhibitor. In some aspects, the additional anti-cancer therapy comprises an antibody or an antigen binding portion thereof that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof. In some aspects, the anti-PD-1 antibody comprises nivolumab or pembrolizumab.

### BRIEF DESCRIPTION OF THE DRAWINGS/FIGURES

FIGS. 1A-1B are graphical representations of titration of anti-integrin-αvβ1 clones 10392 (FIG. 1A) and clone 10404 (FIG. 1B).

FIGS. 2A-2B are bar graphs summarizing epitope binning of the anti-integrin-αvβ1 Fab 10404 (FIG. 2A) and Fab 10392 (FIG. 2B). All numbers on top of bar graphs represent percent values, MFI indicates mean fluorescence from the mean, and error bars indicate SD.

FIGS. 3A-3B are scatter plots showing IgG affinity titration validations of the anti-integrin-αvβ1 Fab 10392 (FIG. 3A) and Fab 10404 (FIG. 3B) for the different integrin CHO cell lines. MFI indicates mean fluorescence from the mean, and error bars indicate SD.

FIG. 4 is a scatter plot showing IgG affinity titration validations of the different modalities of Ab clones for recombinant human integrin-αvβ1.

FIG. 5 is a scatter plot showing IgG affinity titration validations of the different modalities of Ab clones for recombinant mouse integrin-αvβ1.

FIGS. 6A-6C present size exclusion chromatography graphs of IgG clones 10392 (FIG. 6A), 10404 (FIG. 6B), and 11867 (FIG. 6C).

FIGS. 7A-7B are PAGE-SDS gel images showing assessment of IgG purity post expression and purification for IgG clones 10404 (FIG. 7A), 10392 (FIG. 7B).

FIGS. 8A-8B are bar graphs illustrating freeze-thaw analysis of clone 11867 and different IgG modalities. FIG. 8A provides a graph showing concentration values determined by spectrophotometry at absorption 280 wavelength. FIG. 8B provides a graph showing flow-cytometry mean fluorescent intensities (MFI) values. IgG binding activity was measured using AvB1 CHO cells, and all Samples Normalized to “No Treatment” of each group.

FIG. 9 is a Bar graph summary of integrin-αvβ1 CHO cellular adhesion for LAP-TGFβ in the presence of clone IgGs 10404, 10392, and 10404/10392.

FIG. 10A is an illustration of the different bi-specific IgGs, where null indicates a non-selective (random CDRs) bi-IgG arm. FIG. 10B is a bar graph summary of Fab binding activity for integrin-αvβ1 CHO cells in the presence of bi-specific IgG 10404/10392. FIG. 10C is a bar graph summary of bi-specific IgGs binding activity for integrin-αvβ1 CHO cells in the presence of different Fab clones. FIG. 10D is a bar graph summary of integrin-αvβ1 CHO cellular adhesion for LAP-TGFβ in the presence of different clone IgGs. FIG. 10E is a titration graph for integrin-αvβ1 CHO cells by flow-cytometry measurements. FIG. 10F is a graph of inhibitory titration of integrin-αvβ1 CHO cellular for adhesion for LAP-TGFβ. MFI indicates mean fluorescence from the mean, and error bars indicate SD.

FIGS. 11A-11F show data of comparison of inhibitory properties from the different 10404 and 10392 clone IgG modalities. FIG. 11A is a table summary of inhibitory values for cellular adhesion to LAP-TGFβ of different clone IgGs using different integrin CHO cell lines. FIGS. 11B-11F are graph summaries of inhibitory titration curves for cellular adhesion to LAP-TGFβ of clone IgGs αvβ1 (FIG. 11B), αvβ6 (FIG. 11C), αvβ3 (FIG. 11D), αvβ8 (FIG. 11E), and αvβ5 (FIG. 11F) using different integrin CHO cell lines. Error bars indicate SD.

FIG. 12A is a bar graph summary showing epitope binding competition of the different IgG Abs in the presence of 10404 Fab protein using integrin-αvβ1 CHO cells. FIG. 12B is a bar graph summary of IgG inhibition for cellular adhesion to LAP-TGFβ of different using different integrin CHO cell lines. For each cell line, the different IgG Abs used in the experiments (11883, 11855, 11857, 11874, 11914, and 11867) are shown from left to right. FIGS. 12C-12I are size exclusion chromatography graphs of traztuzumab (FIG. 12C), 10404 (FIG. 12D), 11855 (FIG. 12E), 11857 (FIG. 12F), 11867 (FIG. 12G), 11883 (FIG. 12H), and 11914 (FIG. 12I) IgG clones. FIG. 12J is a table summary of affinity and inhibitory values the different clone IgGs. FIG. 12K is a titration graph for integrin-αvβ1 CHO cells by flow-cytometry measurements. FIG. 12L is a graph of inhibitory titration of integrin-αvβ1 CHO cellular for adhesion for LAP-TGFβ using different clone IgGs. MFI indicates mean fluorescence from the mean, and error bars indicate SD.

FIGS. 13A and 13C-13F are graph summaries show titration curves for the 11867 and 10392 IgG modalities against the integrin CHO cell lines αvβ1 (FIG. 13A), αvβ3 (FIG. 13C), αvβ5 (FIG. 13D), αvβ6 (FIG. 13E), and αvβ8 (FIG. 13F). MFI indicates mean fluorescence from the mean, and error bars indicate SD. FIG. 13B is a table summary of affinity values for different integrin CHO cell lines.

FIGS. 14A and 14C-14F are graph summaries of inhibitory titration curves for cellular adhesion to LAP-TGFβ of different clone IgGs using the integrin CHO cell lines cell lines αvβ1 (FIG. 14A), αvβ3 (FIG. 14C), αvβ5 (FIG. 14D), αvβ6 (FIG. 14E), and αvβ8 (FIG. 14F). Error bars indicate SD. FIG. 14B is a table summary of inhibitory values for cellular adhesion to LAP-TGFβ of different clone IgGs using different integrin CHO cell lines.

FIG. 15A is a graph of flow-cytometry summary of αv- and β1-subunit expression levels using 11876/10392 bi-IgG, showing log 2 values of fold-change MFI signals of subunit selective versus isotype control antibodies. FIG. 15B is a bar graph summary of lung cellular proliferation at endpoint day 4 in the presence of 11876/10392 bi-IgG. FIGS. 15C-15G are time course graphs showing cellular proliferation of lung cancer cell lines A549 (FIG. 15C), H292 (FIG. 15D), H661 (FIG. 15E), H460 (FIG. 15F), and H1563 (FIG. 15G) in the presence of 11876/10392 bi-IgG. Error bars indicate SD.

FIG. 16A is a titration curve graph illustrating inhibitory titration assessment on different lung cancer cell lines using 11867/10392 bi-IgG. Error bars indicate SD. FIG. 16B Table summary of IC50 values from bi-IgG 11867/10392 on different lung cancer cell lines.

FIG. 17A-17P are graphical representations of assessment of cell migration inhibition using bi-IgG 11867/10392. FIGS. 17A (A549), 17E (H460), 17I (H661), and 17M (H1563) are time lapse graphs of wound healing assay using different lung cancer lines, and error bars indicate SD. FIGS. 17B, 17F, 17J, and 17N show representative micrographs of untreated cells; FIGS. 17C, 17G, 17K, and 17O show representative micrographs of control cells; and FIGS. 17D, 17H, 17L, and 17P show representative micrographs of bi-IgG 11867/10392 treated cells for each experiment.

### DETAILED DESCRIPTION OF DISCLOSURE

The present disclosure relates to antibodies, bispecific antibodies, multispecific antibodies, and antigen binding fragments thereof that specifically bind an integrin-αv heterodimer and inhibits integrin-αv-mediated activation of TGFβ. Some aspects of the present disclosure are directed to an antibody or an antigen-binding portion thereof that specifically binds an integrin-αv heterodimer and inhibits integrin-αv-mediated activation of TGFβ. Some aspects of the present disclosure are directed to a bispecific antibody or an antigen-binding portion thereof that specifically binds an integrin-αv heterodimer and inhibits integrin-αv-mediated activation of TGFβ. In some aspects, the bispecific antibody or the antigen-biding portion thereof comprises at least a first paratope and a second paratope, wherein the first paratope binds a first epitope on an integrin αv heterodimer (e.g., integrin-αvβ1) heterodimer, and wherein the second paratope binds a second epitope on the integrin αv heterodimer (e.g., integrin-αvβ1) heterodimer. Other aspects of the present disclosure relate to methods of treating a subject in need thereof, comprising administering to the subject an antibody, a bispecific antibody, a multispecific antibody, or antigen-binding fragment thereof that specifically binds an integrin-αv heterodimer and inhibits integrin-αv-mediated activation of TGFβ. In some aspects, the subject has a cancer, and the antibody, a bispecific antibody, a multispecific antibody, or antigen-binding fragment thereof treats the cancer in the subject.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). The term “bispecific antibody,” as used herein, refers to an antibody comprising at least two antigen-binding domains, i.e., at least two paratopes. As such, in some aspects, a bispecific antibody comprises at least two heavy chain variable regions (VH1 and VH2) and at least two light chain variable regions (VL1 and VL2. In some aspects, the at least two heavy chain variable regions are the same or different. In some aspects, the at least two light chain variable regions are the same or different. A “multispecific antibody,” as used herein, refers to an antibody comprising at least three antigen-binding domains, i.e., at least three paratopes.

In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an anti-integrin-αv heterodimer IgG1, IgG2, IgG3 or IgG4 antibody consists of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷M or less, 10⁻⁸ M or less, 5×10⁻⁹M or less, or between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to human integrin αv heterodimer (e.g., integrin-αvβ1) can, in some aspects, also have cross-reactivity with integrin αv heterodimer (e.g., integrin-αvβ1) antigens from certain primate species (e.g., cynomolgus integrin αv heterodimer (e.g., integrin-αvβ1)), but cannot cross-react with integrin αv heterodimer (e.g., integrin-αvβ1) antigens from other species or with an antigen other than integrin αv heterodimer (e.g., integrin-αvβ1).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the anti-integrin-αv heterodimer antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human integrin αv heterodimer (e.g., integrin-αvβ1)). It has been shown that the antigen-binding function of an antibody can be performed by fragments of a full-length antibody. Examples of binding fragments encompassed within the term “antigen-binding portion” of an antibody, e.g., an anti-integrin-αv heterodimer antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). In some aspects, a bispecific antibody disclosed herein is produced by modifying a first heavy chain in a way that increases the affinity of the first heavy chain for a second heavy chain, e.g., through alteration of the amino acid sequence or one or more amino acid side chains in the constant region of a first heavy chain. In some aspects, modifications are made to both the first heavy chain and the second heavy chain. In some aspects, the modifications facilitate heterodimerization of the first and second heavy chains. Any modification that increases the affinity of the first and second heavy chains can be used. In certain aspects, the first heavy chain is modified to comprise a knob motif, and the second heavy chain is modified to comprise a hole motif.

The term “monoclonal antibody,” as used herein, refers to an antibody from a population of substantially homogeneous antibodies, i.e., the individual antibodies comprised in the population are substantially similar and bind the same epitope(s) (e.g., the antibodies display a single binding specificity and affinity), except for possible variants that may arise during production of the monoclonal antibody, such variants generally being present in minor amounts. The modifier “monoclonal” indicates the character of the antibody as being obtained from a substantially homogeneous population of antibodies, and is not to be construed as requiring production of the antibody by any particular method. The term “human monoclonal antibody” refers to an antibody from a population of substantially homogeneous antibodies that display(s) a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences encoded by the germline genes, but include subsequent rearrangements and mutations which occur, for example, during antibody maturation. As known in the art (see, e.g., Lonberg (2005) Nature Biotech. 23(9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The anti-integrin-αv heterodimer antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In some aspects of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally-occurring variants within a specific isotype group, which variants differ in a few amino acids (see, e.g., Jefferis et al. (2009) mAbs 1:1). Anti-integrin-αv heterodimer antibodies described herein can be of any allotype. As used herein, antibodies referred to as “IgG1f,” “IgG1.1f,” or “IgG1.3f” isotype are IgG1, effectorless IgG1.1, and effectorless IgG1.3 antibodies, respectively, of the allotype “f,” i.e., having 214R, 356E and 358M according to the EU index as in Kabat.

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other proteins and cellular material.

As used herein, an antibody that “binds integrin αv heterodimer (e.g., integrin-αvβ1)” is intended to refer to an antibody that interacts with integrin αv heterodimer (e.g., integrin-αvβ1), e.g., in binding assays using CHO cells transfected with human integrin αv heterodimer (e.g., integrin-αvβ1) or integrin αv heterodimer (e.g., integrin-αvβ1) expressing tumor cells, with an EC₅₀ of about 25 μg/mL or less, about 23 μg/mL or less, about 20 μg/mL or less, about 15 μg/mL or less, about 10 μg/mL or less, about 5 μg/mL or less, about 3 μg/mL or less, about 2 μg/mL or less, about 1 μg/mL or less, about 0.5 μg/mL or less, about 0.45 μg/mL or less, about 0.4 μg/mL or less, about 0.35 μg/mL or less, or about 0.3 μg/mL or less, in art-recognized methods. In some aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of about 200 nM or less, about 175 nM or less, about 160 nM or less, about 150 nM or less, about 125 nM or less, about 110 nM or less, about 100 nM or less about 80 nM or less, about 75 nM or less, about 60 nM or less, about 50 nM or less, about 40 nM or less, about 35 nM or less, about 30 nM or less, about 25 nM or less, about 20 nM or less, about 15 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1.9 nM or less, about 1.8 nM or less, about 1.7 nM or less, about 1.6 nM or less, about 1.5 nM or less, about 1.4 nM or less, about 1.3 nM or less, about 1.2 nM or less, about 1.1 nM or less, about 1.0 nM or less, about 0.9 nM or less, about 0.8 nM or less, about 0.7 nM or less, about 0.6 nM or less, about 0.5 nM or less, about 0.4 nM or less, about 0.3 nM or less, about 0.2 nM or less, or about 0.1 nM or less. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 10 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 5 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 4 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 3.5 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 3 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 2.5 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 2.4 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 2.3 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 2.1 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 2.0 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.9 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.8 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.7 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.6 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.5 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.4 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.3 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.2 nM. In certain aspects, the anti-integrin αv heterodimer (e.g., integrin-αvβ1) antibody binds human-integrin αv heterodimer (e.g., integrin-αvβ1) expressed on, e.g., CHO cells, with an EC₅₀ of less than about 1.0.

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

The term “epitope” or “antigenic determinant” refers to a site on an antigen (e.g., integrin αv heterodimer (e.g., integrin-αvβ1)) to which an immunoglobulin or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from integrin αv heterodimer (e.g., integrin-αvβ1)) are tested for reactivity with a given antibody (e.g., anti-integrin αv heterodimer (e.g., integrin-αvβ1) antibody). Methods of determining spatial conformation of epitopes include techniques in the art and those described herein, for example, x-ray crystallography, x-ray co-crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on integrin αv heterodimer (e.g., integrin-αvβ1)” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some aspects, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include: competition for binding to cells expressing integrin αv heterodimer (e.g., integrin-αvβ1), e.g., by flow cytometry, such as described in the Examples. Other methods include: SPR (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J Immunol. 32:77 (1990)).

As used herein, the term “paratope” refers to the amino acid or amino acids present in an antibody or an antigen-binding fragment thereof that interact with the epitope on the antigen.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., recombinant human integrin αv heterodimer (e.g., integrin-αvβ1), as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human integrin αv heterodimer (e.g., integrin-αvβ1)” refers to an antibody that binds to human integrin αv heterodimer (e.g., integrin-αvβ1) with a K_{D} of 10⁻⁷ M or less, such as approximately less than 10⁻⁸ M, 10⁻⁹ M or 10⁻¹⁰ M or even lower.

The term “k_{assoc}” or “kₐ”, as used herein, is intended to refer to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e., k_{d}/kₐ) and is expressed as a molar concentration (M). K_{D} values for antibodies can be determined using methods well established in the art. Available methods for determining the K_{D} of an antibody include surface plasmon resonance, a biosensor system such as a BIACORE® system or flow cytometry and Scatchard analysis.

As used herein, the term “high affinity” for an IgG antibody refers to an antibody having a K_{D} of 10⁻⁸M or less, 10⁻⁹M or less, or 10⁻¹⁰M or less for a target antigen. However, “high affinity” binding can vary for other antibody isotypes. For example, “high affinity” binding for an IgM isotype refers to an antibody having a K_{D} of 10⁻¹⁰ M or less, or 10⁻⁸ M or less.

The term “EC₅₀” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an anti-integrin αv heterodimer (e.g., integrin-αvβ1) antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some aspects, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response.

As used herein, the term “linked” refers to the association of two or more molecules. The linkage can be covalent or non-covalent. The linkage also can be genetic (i.e., recombinantly fused). Such linkages can be achieved using a wide variety of art-recognized techniques, such as chemical conjugation and recombinant protein production.

As used herein, “administering” refers to the physical introduction of a composition comprising a therapeutic agent to a subject, using any of the various methods and delivery systems. Different routes of administration for the anti-integrin αv heterodimer (e.g., integrin-αvβ1) antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the phrase “inhibits growth of a tumor” includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%. In some aspects, inhibition of tumor growth is measured as the percent tumor growth inhibition (TGI %). TGI % can be determined by calculating the TGI at dat “t” calculated from all treatment animals according to the formula: [1−((Tₜ/T₀)/(Cₜ/C₀))]/[(Cₜ−C₀)/Cₜ]*100 [Formula 1], where Tₜ=individual tumor size of treated animal at time ‘t’, T₀=individual tumor size of treated animal at first measurement, Cₜ=median tumors size of control animals at time ‘t’, C₀=median tumor size of control animals at first measurement.

As used herein, “cancer” refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods, such as in human subjects during clinical trials, in animal model systems predictive of efficacy in humans, or by assaying the activity of the agent in in vitro assays.

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using the assays described infra. Alternatively, this property of a composition can be evaluated by examining the ability of the compound to inhibit cell growth, such inhibition can be measured in vitro by any assays. In some aspects described herein, tumor regression can be observed and continue for a period of at least about 20 days, at least about 40 days, or at least about 60 days.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “μg” and “μM,” respectively.

Various aspects described herein are described in further detail in the following subsections.

### II. Compositions of the Disclosure

Certain aspects of the present disclosure are directed to isolated antibodies or antigen-binding portions thereof that specifically bind to an integrin-αv heterodimer (e.g., integrin-αvβ1), and inhibit, prevent or reduce integrin-αv-mediated activation of TGFβ. Some aspects of the present disclosure are directed to bispecific antibodies that specifically bind to an integrin-αv heterodimer (e.g., integrin-αvβ1), and inhibit, prevent or reduce integrin-αv-mediated activation of TGFβ. Some aspects of the present disclosure are directed to methods of treating a disease or condition in a subject in need thereof, comprising administering to the subject an antibody or an antigen-binding portion thereof or a bispecific antibody that specifically binds to an integrin-αv heterodimer (e.g., integrin-αvβ1), and inhibits, prevents, or reduces integrin-αv-mediated activation of TGFβ.

In some aspects, integrin-αv-mediated activation of TGFβ is reduced by at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold relative to the level of integrin-αv-mediated activation of TGFβ prior to contact with the antibody or an antigen-binding portion thereof or a bispecific antibody of the disclosure. In some aspects, integrin-αv-mediated activation of TGFβ is completely ablated following contact with an antibody or an antigen-binding portion thereof or a bispecific antibody of the disclosure.

II.A. Anti-Integrin-αv Heterodimer Antibodies

Certain aspects of the present disclosure are directed to isolated antibodies or antigen-binding protions thereof that specifically bind to an integrin-αv heterodimer (e.g., integrin-αvβ1), and inhibiting, preventing or reducing integrin-αv-mediated activation of TGFβ. In some aspects, the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 15. In some aspects, the VH-CDR2 comprises an amino acid sequence of SEQ ID NO: 4, 14, or 24. In some aspects, the VH-CDR1 comprises an amino acid sequence of SEQ ID NO: 3, 13, or 23. In some aspects, the VL-CDR3 comprises an amino acid sequence SEQ ID NO: 10, 20, or 30. In some aspects, the VL-CDR2 comprises an amino acid sequence of SEQ ID NO: 9, 19, or 29. In some aspects, the VL-CDR1 comprises an amino acid sequence of SEQ ID NOs: 8, 18, or 28.

TABLE 1
Anti-integrin-αv heterodimer Antibody Sequences

| SEQ | | |
|---|---|---|
| ID | | |
| NO: | Name | Sequence |
| 1 | pSCSTa-hG1 | EVQLVESGGGLVQPGGSLRLSCAASGFNIYYSSIHWVRQAPGKGLEWVASISSSSG |
| | (10392) heavy | STSTYADSVKGRFTISADTSKNTAYLQMNSLRAEDTAVYYCARSYYGGGYPYAL |
| | chain | DYWGQGTLVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| 2 | pSCSTa-hG1 | EVQLVESGGGLVQPGGSLRLSCAASGFDIYYSSIHWVRQAPGKGLEWVASISSSSG |
| | (10392) VH | STSTYADSVKGRFTISADTSKNTAYLQMNSLRAEDTAVYYCARSYYGGGYPYAL |
| | | DYWGQGTLVTVSS |
| 3 | pSCSTa-hG1 | IYYSSI |
| | (10392) VH- | |
| | CDR1 | |
| 4 | pSCSTa-hG1 | SISSSSGSTST |
| | (10392) VH- | |
| | CDR2 | |
| 5 | pSCSTa-hG1 | SYYGGGYPYAL |
| | (10392) VH- | |
| | CDR3 | |
| 6 | pSCSTa-hK | DIQMTQSPSSLSASVGDRVTITCRASQSVSSAVAWYQQKPGKAPKLLIYSASSLYS |
| | (10392) light | GVPSRFSGSRSGTDFTLTISSLQPEDFATYYCQQYFGGLITFGQGTKVEIKRTVAAP |
| | chain | SVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 7 | pSCSTa-hK | DIQMTQSPSSLSASVGDRVTITCRASQSVSSAVAWYQQKPGKAPKLLIYSASSLYS |
| | (10392) VL | GVPSRFSGSRSGTDFTLTISSLQPEDFATYYCQQYFGGLITFGQGTKVEIK |
| 8 | pSCSTa-hK | SVSSA |
| | (10392) VL- | |
| | CDR1 | |
| 9 | pSCSTa-hK | SASSLYS |
| | (10392) VL- | |
| | CDR2 | |
| 10 | pSCSTa-hK | YFGGLI |
| | (10392) VL- | |
| | CDR3 | |
| 11 | pSCSTa-hG1 | EVQLVESGGGLVQPGGSLRLSCAASGFNLYSSSIHWVRQAPGKGLEWVAYIYPSY |
| | (10404) heavy | GYTSTYADSVKGRFTISADTSKNTAYLQMNSLRAEDTAVYYCARPAPYYSWYPA |
| | chain | MDYWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| 12 | pSCSTa-hG1 | EVQLVESGGGLVQPGGSLRLSCAASGFNLYSSSIHWVRQAPGKGLEWVAYIYPSY |
| | (10404) VH | GYTSTYADSVKGRFTISADTSKNTAYLQMNSLRAEDTAVYYCARPAPYYSWYPA |
| | | MDYWGQGTLVTVSS |
| 13 | pSCSTa-hG1 | LYSSSI |
| | (10404) VH- | |
| | CDR1 | |
| 14 | pSCSTa-hG1 | YIYPSYGYTST |
| | (10404) VH- | |
| | CDR2 | |
| 15 | pSCSTa-hG1 | PAPYYSWYPAM |
| | (10404) VH- | |
| | CDR3 | |
| 16 | pSCSTa-hK | DIQMTQSPSSLSASVGDRVTITCRASQSVSSAVAWYQQKPGKAPKLLIYSASSLYS |
| | (10404) light | GVPSRFSGSRSGTDFTLTISSLQPEDFATYYCQQASGSPFTFGQGTKVEIKRTVAAP |
| | chain | SVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 17 | pSCSTa-hK | DIQMTQSPSSLSASVGDRVTITCRASQSVSSAVAWYQQKPGKAPKLLIYSASSLYS |
| | (10404) VL | GVPSRFSGSRSGTDFTLTISSLQPEDFATYYCQQASGSPFTFGQGTKVEIK |
| 18 | pSCSTa-hK | SVSSA |
| | (10404) VL- | |
| | CDR1 | |
| 19 | pSCSTa-hK | SASSLYS |
| | (10404) VL- | |
| | CDR2 | |
| 20 | pSCSTa-hK | ASGSPF |
| | (10404) VL- | |
| | CDR3 | |
| 21 | pSCSTa-hG1 | EVQLVESGGGLVQPGGSLRLSCAASGFDFDSYAIHWVRQAPGKGLEWVAYFYPG |
| | (11867) heavy | HDYSTYADSVKGRFTISADTSKNTAYLQMNSLRAEDTAVYYCARPSPYFSWHQA |
| | chain | MDYWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| 22 | pSCSTa-hG1 | EVQLVESGGGLVQPGGSLRLSCAASGFDFDSYAIHWVRQAPGKGLEWVAYFYPG |
| | (11867) VH | HDYSTYADSVKGRFTISADTSKNTAYLQMNSLRAEDTAVYYCARPSPYFSWHQA |
| | | MDYWGQGTLVTVSS |
| 23 | pSCSTa-hG1 | FDSYAI |
| | (11867) VH- | |
| | CDR1 | |
| 24 | pSCSTa-hG1 | YFYPGHDYST |
| | (11867) VH- | |
| | CDR2 | |
| 25 | pSCSTa-hG1 | PSPYFSWHQAM |
| | (11867) VH- | |
| | CDR3 | |
| 26 | pSCSTa-hK | DIQMTQSPSSLSASVGDRVTITCRASQSVSSAVAWYQQKPGKAPKLLIYSASSLYS |
| | (11867) light | GVPSRFSGSRSGTDFTLTISSLQPEDFATYYCQQATGSEVTFGQGTKVEIKRTVAAP |
| | chain | SVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 27 | pSCSTa-hK | DIQMTQSPSSLSASVGDRVTITCRASQSVSSAVAWYQQKPGKAPKLLIYSASSLYS |
| | (11867) VL | GVPSRFSGSRSGT |
| 28 | pSCSTa-hK | SVSSA |
| | (11867) VL- | |
| | CDR1 | |
| 29 | pSCSTa-hK | SASSLYS |
| | (11867) VL- | |
| | CDR2 | |
| 30 | pSCSTa-hK | ATGSEV |
| | (11867) VL- | |
| | CDR3 | |
| 31 | pSCSTa- | EVQLVESGGGLVQPGGSLRLSCAASGFNIYYSSIHWVRQAPGKGLEWVASISSSSG |
| | Fck79 (10392) | STSYADSVKGRFTISADTSKNTAYLQMNSLRAEDTAVYYCARSYYGGGYPYALD |
| | knob heavy | YWGQGTLVTVSSASTKGPSVFPLAPSSKSTSGGTAALGCLVKDYFPEPVTVSWNS |
| | chain | GALTSGVHTFPAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSN |
| | | KALPAPIEKTISKAKGQPREPMVFDLPPSREEMTKNQVSLWCMVKGFYPSDIAVE |
| | | WESNGQPENNYKTTPPVLDSDGSFFLYSKLTVDKSRWQQGNVFSCSVMHEALHN |
| | | HYTQKSLSLSPGK |
| 32 | pSCSTa- | EVQLVESGGGLVQPGGSLRLSCAASGFNIYYSSIHWVRQAPGKGLEWVASISSSSG |
| | Fck79 (10392) | STSYADSVKGRFTISADTSKNTAYLQMNSLRAEDTAVYYCARSYYGGGYPYALD |
| | knob VH | YWGQGTLVTVSS |
| 33 | pSCSTa- | ASTKGPSVFPLAPSSKSTSGGTAALGCLVKDYFPEPVTVSWNSGALTSGVHTFPAV |
| | Fck79 (10392) | LQSSGLYSLSSVVTVPSSSLGTQTYICNVNHKPSNTKVDKKVEPKSCDKTHTCPPC |
| | knob Constant | PAPELLGGPSVFLFPPKPKDTLMISRTPEVTCVVVDVSHEDPEVKFNWYVDGVEV |
| | Heavy | HNAKTKPREEQYNSTYRVVSVLTVLHQDWLNGKEYKCKVSNKALPAPIEKTISKA |
| | | KGQPREPMVFDLPPSREEMTKNQVSLWCMVKGFYPSDIAVEWESNGQPENNYKT |
| | | TPPVLDSDGSFFLYSKLTVDKSRWQQGNVFSCSVMHEALHNHYTQKSLSLSPGK |
| 34 | pSCSTa Fch8c | EVQLVESGGGLVQPGGSLRLSCAASGFNLYSSSIHWVRQAPGKGLEWVAYIYPSY |
| | (10404) hole | GYTSTYADSVKGRFTISADTSKNTAYLQMNSLRAEDTAVYYCARPAPYYSWYPA |
| | heavy chain | MDYWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKEYKCKVS |
| | | NKALPAPIEKTISKAKGQPREPQVYTLPPIRELMTSNQVSLSCAVKGFYPSDIAVEW |
| | | ESNGQPENNYKTTPPVLDSDGSFFLVSKLTVDKSRWQQGNVFSCSVMHEALHNH |
| | | YTQKSLSLSPGK |
| 35 | pSCSTa Fch8c | EVQLVESGGGLVQPGGSLRLSCAASGFNLYSSSIHWVRQAPGKGLEWVAYIYPSY |
| | (10404) hole | GYTSTYADSVKGRFTISADTSKNTAYLQMNSLRAEDTAVYYCARPAPYYSWYPA |
| | VH | MDYWGQGTLVTVSS |
| 36 | pSCSTa Fch8c | ASTKGPSVFPLAPSSKSTSGGTAALGCLVKDYFPEPVTVSWNSGALTSGVHTFPAV |
| | (10404) hole | LQSSGLYSLSSVVTVPSSSLGTQTYICNVNHKPSNTKVDKKVEPKSCDKTHTCPPC |
| | Constant | PAPELLGGPSVFLFPPKPKDTLMISRTPEVTCVVVDVSHEDPEVKFNWYVDGVEV |
| | Heavy | HNAKTKPREEQYNSTYRVVSVLTVLHQDWLNGKEYKCKVSNKALPAPIEKTISKA |
| | | KGQPREPQVYTLPPIRELMTSNQVSLSCAVKGFYPSDIAVEWESNGQPENNYKTTP |
| | | PVLDSDGSFFLVSKLTVDKSRWQQGNVFSCSVMHEALHNHYTQKSLSLSPGK |
| 37 | pSCSTa Fch8c | EVQLVESGGGLVQPGGSLRLSCAASGFDFDSYAIHWVRQAPGKGLEWVAYFYPG |
| | (11867) hole | HDYSTYADSVKGRFTISADTSKNTAYLQMNSLRAEDTAVYYCARPSPYFSWHQA |
| | heavy chain | MDYWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKEYKCKVS |
| | | NKALPAPIEKTISKAKGQPREPQVYTLPPIRELMTSNQVSLSCAVKGFYPSDIAVEW |
| | | ESNGQPENNYKTTPPVLDSDGSFFLVSKLTVDKSRWQQGNVFSCSVMHEALHNH |
| | | YTQKSLSLSPGK |
| 38 | pSCSTa Fch8c | EVQLVESGGGLVQPGGSLRLSCAASGFDFDSYAIHWVRQAPGKGLEWVAYFYPG |
| | (11867) hole | HDYSTYADSVKGRFTISADTSKNTAYLQMNSLRAEDTAVYYCARPSPYFSWHQA |
| | VH | MDYWGQGTLVTVSS |
| 39 | pSCSTa Fch8c | ASTKGPSVFPLAPSSKSTSGGTAALGCLVKDYFPEPVTVSWNSGALTSGVHTFPAV |
| | (11867) hole | LQSSGLYSLSSVVTVPSSSLGTQTYICNVNHKPSNTKVDKKVEPKSCDKTHTCPPC |
| | Constant | PAPELLGGPSVFLFPPKPKDTLMISRTPEVTCVVVDVSHEDPEVKFNWYVDGVEV |
| | Heavy | HNAKTKPREEQYNSTYRVVSVLTVLHQDWLNGKEYKCKVSNKALPAPIEKTISKA |
| | | KGQPREPQVYTLPPIRELMTSNQVSLSCAVKGFYPSDIAVEWESNGQPENNYKTTP |
| | | PVLDSDGSFFLVSKLTVDKSRWQQGNVFSCSVMHEALHNHYTQKSLSLSPGK |

Some aspects of the present disclosure are directed to an antibody or antigen binding portion thereof that cross competes with a reference antibody or antigen-binding portion thereof for binding to an integrin-αv heterodimer, wherein the reference antibody comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 15.

Some aspects of the present disclosure are directed to an antibody or antigen binding portion thereof that binds the same epitope on an integrin-αv heterodimer as a reference antibody or antigen-binding portion thereof, wherein the reference antibody comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 15.

Some aspects of the present disclosure are directed to an antibody or antigen binding portion thereof that binds an epitope on an integrin-αv heterodimer that has at least one amino acid residue that overlaps with an epitope that is recognized by a reference antibody or antigen-binding portion thereof, wherein the reference antibody comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 15.

In some aspects, the VH-CDR2 of the reference antibody comprises an amino acid sequence of SEQ ID NO: 4, 14, or 24. In some aspects, the VH-CDR1 of the reference antibody comprises an amino acid sequence of SEQ ID NO: 3, 13, or 23. In some aspects, the VL-CDR3 of the reference antibody comprises an amino acid sequence SEQ ID NO: 10, 20, or 30. In some aspects, the VL-CDR2 of the reference antibody comprises an amino acid sequence of SEQ ID NO: 9, 19, or 29. In some aspects, the VL-CDR1 of the reference antibody comprises an amino acid sequence of SEQ ID NOs: 8, 18, or 28.

In some aspects, the reference antibody or antigen-binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR1 comprises the amino acid sequence set forth in SEQ ID NO: 3; the VH-CDR2 comprises the amino acid sequence set forth in SEQ ID NO: 4; the VH-CDR3 comprises the amino acid sequence set forth in SEQ ID NO: 5; the VL-CDR1 comprises the amino acid sequence set forth in SEQ ID NO: 8; the VL-CDR2 comprises the amino acid sequence set forth in SEQ ID NO: 9; and the VL-CDR3 comprises the amino acid sequence set forth in SEQ ID NO: 10.

In some aspects, the reference antibody or antigen-binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR1 comprises the amino acid sequence set forth in SEQ ID NO: 13; the VH-CDR2 comprises the amino acid sequence set forth in SEQ ID NO: 14; the VH-CDR3 comprises the amino acid sequence set forth in SEQ ID NO: 15; the VL-CDR1 comprises the amino acid sequence set forth in SEQ ID NO: 18; the VL-CDR2 comprises the amino acid sequence set forth in SEQ ID NO: 19; and the VL-CDR3 comprises the amino acid sequence set forth in SEQ ID NO: 20.

In some aspects, the reference antibody or antigen-binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR1 comprises the amino acid sequence set forth in SEQ ID NO: 23; the VH-CDR2 comprises the amino acid sequence set forth in SEQ ID NO: 24; the VH-CDR3 comprises the amino acid sequence set forth in SEQ ID NO: 25; the VL-CDR1 comprises the amino acid sequence set forth in SEQ ID NO: 28; the VL-CDR2 comprises the amino acid sequence set forth in SEQ ID NO: 29; and the VL-CDR3 comprises the amino acid sequence set forth in SEQ ID NO: 30.

In some aspects, the reference antibody or antigen-binding portion thereof comprises a heavy chain variable region (“VH”) and a light chain variable region (“VL”); wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 2, 12, and 22. In some aspects, the reference antibody comprises an amino acid sequence selected from SEQ ID NOs: 2, 12, and 22. In some aspects, the reference antibody or antigen-binding portion thereof comprises a heavy chain variable region (“VH”) and a light chain variable region (“VL”); wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, and 27. In some aspects, the reference antibody or antigen-binding portion thereof comprises an amino acid sequence selected from SEQ ID NOs: 7, 17, and 27.

In some aspects, the reference antibody or antigen-binding portion thereof comprises a VH comprising the amino acid sequence set forth in SEQ ID NO: 2, and a VL comprising the amino acid sequence set forth in SEQ ID NO: 7. In some aspects, the reference antibody or antigen-binding portion thereof comprises a VH comprising the amino acid sequence set forth in SEQ ID NO: 12, and a VL comprising the amino acid sequence set forth in SEQ ID NO: 17. In some aspects, the reference antibody or antigen-binding portion thereof comprises a VH comprising the amino acid sequence set forth in SEQ ID NO: 22, and a VL comprising the amino acid sequence set forth in SEQ ID NO: 27.

Some aspects of the present disclosure are directed to an isolated antibody or antigen-binding portion thereof that specifically binds to an integrin-αv heterodimer, wherein the antibody or antigen binding portion thereof comprises a VH comprising a VH-CDR 1, a VH-CDR2, and a VH-CDR3 and a VL comprising VL-CDR1, VL-CDR2, and VL-CDR3; wherein the VH-CDR1 comprises the amino acid sequence set forth in SEQ ID NO: 3; the VH-CDR2 comprises the amino acid sequence set forth in SEQ ID NO: 4; the VH-CDR3 comprises the amino acid sequence set forth in SEQ ID NO: 5; the VL-CDR1 comprises the amino acid sequence set forth in SEQ ID NO: 8; the VL-CDR2 comprises the amino acid sequence set forth in SEQ ID NO: 9; and the VL-CDR3 comprises the amino acid sequence set forth in SEQ ID NO: 10.

Some aspects of the present disclosure are directed to an isolated antibody or antigen-binding portion thereof that specifically binds to an integrin-αv heterodimer, wherein the antibody or antigen binding portion thereof comprises a VH comprising a VH-CDR 1, a VH-CDR2, and a VH-CDR3 and a VL comprising VL-CDR1, VL-CDR2, and VL-CDR3; wherein the VH-CDR1 comprises the amino acid sequence set forth in SEQ ID NO: 13; the VH-CDR2 comprises the amino acid sequence set forth in SEQ ID NO: 14; the VH-CDR3 comprises the amino acid sequence set forth in SEQ ID NO: 15; the VL-CDR1 comprises the amino acid sequence set forth in SEQ ID NO: 18; the VL-CDR2 comprises the amino acid sequence set forth in SEQ ID NO: 19; and the VL-CDR3 comprises the amino acid sequence set forth in SEQ ID NO: 20.

Some aspects of the present disclosure are directed to an isolated antibody or antigen-binding portion thereof that specifically binds to an integrin-αv heterodimer, wherein the antibody or antigen binding portion thereof comprises a VH comprising a VH-CDR 1, a VH-CDR2, and a VH-CDR3 and a VL comprising VL-CDR1, VL-CDR2, and VL-CDR3; wherein the VH-CDR1 comprises the amino acid sequence set forth in SEQ ID NO: 23; the VH-CDR2 comprises the amino acid sequence set forth in SEQ ID NO: 24; the VH-CDR3 comprises the amino acid sequence set forth in SEQ ID NO: 25; the VL-CDR1 comprises the amino acid sequence set forth in SEQ ID NO: 28; the VL-CDR2 comprises the amino acid sequence set forth in SEQ ID NO: 29; and the VL-CDR3 comprises the amino acid sequence set forth in SEQ ID NO: 30.

In some aspects, the antibody or antigen-binding portion thereof comprises a VH comprising the amino acid sequence set forth in SEQ ID NO: 2, and a VL comprising the amino acid sequence set forth in SEQ ID NO: 7. In some aspects, the antibody or antigen-binding portion thereof comprises a VH comprising the amino acid sequence set forth in SEQ ID NO: 12, and a VL comprising the amino acid sequence set forth in SEQ ID NO: 17. In some aspects, the antibody or antigen-binding portion thereof comprises a VH comprising the amino acid sequence set forth in SEQ ID NO: 22, and a VL comprising the amino acid sequence set forth in SEQ ID NO: 27.

In some aspects, the antibody or antigen-binding portion thereof comprises a heavy chain (“HC”) and a light chain (“LC”); wherein the HC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 11, and 21. In some aspects, the HC comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, or at least about 50 point mutations relative to the amino acid sequence set forth in SEQ ID NO: 1, 11, or 21. In some aspects, the HC comprises 1 point mutation relative to the amino acid sequence set forth in SEQ ID NO: 1, 11, or 21. In some aspects, the HC comprises 2 point mutations relative to the amino acid sequence set forth in SEQ ID NO: 1, 11, or 21. In some aspects, the HC comprises 3 point mutations relative to the amino acid sequence set forth in SEQ ID NO: 1, 11, or 21. In some aspects, the HC comprises 4 point mutations relative to the amino acid sequence set forth in SEQ ID NO: 1, 11, or 21. In some aspects, the HC comprises 5 point mutations relative to the amino acid sequence set forth in SEQ ID NO: 1, 11, or 21. In some aspects, the HC comprises 10 point mutations relative to the amino acid sequence set forth in SEQ ID NO: 1, 11, or 21.

In some aspects, the HC comprises an amino acid sequence selected from SEQ ID NOs: 1, 11, and 21. In certain aspects, the HC comprises the amino acid sequence set forth in SEQ ID NO: 1. In certain aspects, the HC comprises the amino acid sequence set forth in SEQ ID NO: 11. In certain aspects, the HC comprises the amino acid sequence set forth in SEQ ID NO: 21.

In some aspects, the antibody or antigen-binding portion thereof comprises a HC and a LC; wherein the LC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 6, 16, and 26. In some aspects, the LC comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, or at least about 50 point mutations relative to the amino acid sequence set forth in SEQ ID NO: 6, 16, or 26. In some aspects, the LC comprises 1 point mutation relative to the amino acid sequence set forth in SEQ ID NO: 6, 16, or 26. In some aspects, the LC comprises 2 point mutations relative to the amino acid sequence set forth in SEQ ID NO: 6, 16, or 26. In some aspects, the LC comprises 3 point mutations relative to the amino acid sequence set forth in SEQ ID NO: 6, 16, or 26. In some aspects, the LC comprises 4 point mutations relative to the amino acid sequence set forth in SEQ ID NO: 6, 16, or 26. In some aspects, the LC comprises 5 point mutations relative to the amino acid sequence set forth in SEQ ID NO: 6, 16, or 26. In some aspects, the LC comprises 10 point mutations relative to the amino acid sequence set forth in SEQ ID NO: 6, 16, or 26.

In some aspects, the LC comprises an amino acid sequence selected from SEQ ID NOs: 1, 11, and 21. In certain aspects, the LC comprises the amino acid sequence set forth in SEQ ID NO: 6. In certain aspects, the LC comprises the amino acid sequence set forth in SEQ ID NO: 16. In certain aspects, the LC comprises the amino acid sequence set forth in SEQ ID NO: 26.

In some aspects, the antibody or antigen-binding portion thereof comprises a HC and a LC; wherein the HC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 11, and 21; and wherein the LC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 6, 16, and 26. In some aspects, the antibody or antigen-binding portion thereof comprises a HC and a LC; wherein the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 1; and wherein the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 6. In some aspects, the antibody or antigen-binding portion thereof comprises a HC and a LC; wherein the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11; and wherein the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the antibody or antigen-binding portion thereof comprises a HC and a LC; wherein the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 21; and wherein the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26.

In some aspects, the antibody or antigen-binding portion thereof comprises a HC and a LC; wherein the HC comprises the amino acid sequence set forth in SEQ ID NO: 1; and wherein the LC comprises the amino acid sequence set forth in SEQ ID NO: 6. In some aspects, the antibody or antigen-binding portion thereof comprises a HC and a LC; wherein the HC comprises the amino acid sequence set forth in SEQ ID NO: 11; and wherein the LC comprises the amino acid sequence set forth in SEQ ID NO: 16. In some aspects, the antibody or antigen-binding portion thereof comprises a HC and a LC; wherein the HC comprises the amino acid sequence set forth in SEQ ID NO: 21; and wherein the LC comprises the amino acid sequence set forth in SEQ ID NO: 26.

In some aspects, the antibody is an antigen-binding portion of an antibody. In some aspects, the antigen-binding portion is a Fab, Fab′, F(ab′)2, single chain Fv (scFv), disulfide linked Fv, IgNar, intrabody, IgGΔCH2, minibody, F(ab′)3, tetrabody, triabody, diabody, single-domain antibody, DVD-Ig, Fcab, mAb2, (scFv)2, or scFv-Fc.

In some aspects, the antibody or antigen-binding portion thereof reduces or inhibits tumor cell proliferation. In some aspects, the antibody or antigen-binding portion thereof reduces or inhibits tumor cell migration. In some aspects, the antibody or antigen-binding portion thereof reduces or inhibits tumor cell metastasis. In some aspects, the antibody or antigen-binding portion thereof increases tumor cell death. In some aspects, the antibody or antigen-binding portion thereof is capable of inhibiting tumor growth in a subject in need thereof. In some aspects, the antibody or antigen-binding portion thereof is capable of reducing tumor volume in a subject in need thereof. In some aspects, the antibody or antigen-binding portion thereof is capable of increasing progression-free survival in a subject in need thereof. In some aspects, the antibody or antigen-binding portion thereof is capable of increasing overall survival in a subject in need thereof.

In some aspects, anti-integrin αv heterodimer antibodies described herein bind to human integrin αv heterodimer (e.g., integrin-αvβ1) with high affinity, for example, with a K_{D} of 10⁻⁶ M or less, 10⁻⁷ M or less, 10⁻⁸ M or less, 10⁻⁹ M or less, 10⁻¹⁰ M or less, 10⁻¹¹ M or less, 10⁻¹²M or less, 10⁻¹²M to 10⁻⁷M, 10⁻¹¹ M to 10⁻⁷M, 10⁻¹⁰ M to 10⁻⁷M, or 10⁻⁹ M to 10⁻⁷ M. In some aspects, the anti-integrin αv heterodimer antibody binds to human integrin αv heterodimer (e.g., integrin-αvβ1), e.g., as determined by Surface Plasmon Resonance, e.g. using BIACORE™ (e.g., as described in the Examples), with a K_{D} of 10⁻⁶ M or less, 10⁻⁷ M or less, 10⁻⁸ M or less, 10⁻⁹ M (1 nM) or less, 10⁻¹⁰ M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, or 10⁻⁸ M to 10⁻⁷ M. In some aspects, an anti-integrin αv heterodimer (e.g., integrin-αvβ1) antibody binds to human integrin αv heterodimer (e.g., integrin-αvβ1), e.g., as determined by ELISA, e.g., an integrin αv heterodimer (e.g., integrin-αvβ1) ELISA kit, with an EC₅₀ of EC₅₀ of 100 nM or less, 10 nM or less, 1 nM or less, 100 nM to 0.01 nM, 100 nM to 0.1 nM, 100 nM to 1 nM, or 10 nM to 1 nM, or 10 ug/mL or less, 5 ug/mL or less, 1 ug/mL or less, 0.9 ug/mL or less, 0.8 ug/mL or less, 0.7 ug/mL or less, 0.6 ug/mL or less, 0.5 ug/mL or less, 0.4 ug/mL or less, 0.3 ug/mL or less, 0.2 ug/mL or less, 0.1 ug/mL or less, 0.05 ug/mL or less, or 0.01 ug/mL or less.

In some aspects, the anti-integrin αv heterodimer antibody specifically binds to human integrin αv heterodimer (e.g., integrin-αvβ1) with a K_{D} of about 5×10⁻⁴ M or less, about 1×10⁻⁴ M or less, 5×10⁻⁵ M or less, about 1×10⁻⁵ M or less, about 1×10⁻⁶ M or less, about 1×10⁻⁷ M or less, or about 1×10⁻⁸ M or less, wherein K_{D} is measured by surface plasmon resonance (Biacore) analysis. In some aspects, the anti-integrin αv heterodimer antibody specifically binds human integrin αv heterodimer (e.g., integrin-αvβ1) with an association constant (kₐ) rate of at least about 1×10³ ms⁻¹, at least about 5×10³ ms⁻¹, at least about 1×10⁴ ms⁻¹, at least about 5×10⁴ ms⁻¹, at least about 1×10⁵ ms⁻¹, at least about 5×10⁵ ms⁻¹, or at least about 1×10⁶ ms⁻¹, wherein kₐ is measured by surface plasmon resonance (Biacore) analysis.

In some aspects, the anti-integrin αv heterodimer antibody specifically binds human integrin αv heterodimer (e.g., integrin-αvβ1) with a dissociation constant (k_{d}) rate of about 0.1 s⁻¹ or less, 0.05 s⁻¹ or less, 0.01 s⁻¹ or less, 5×10⁻³ s⁻¹ or less, 1×10⁻³ s⁻¹ or less, 5×10⁻⁴ s⁻¹ or less, 1×10⁻⁴ s⁻¹ or less, 5×10⁻⁵ s⁻¹ or less, or 1×10⁻⁵ s⁻¹ or less, wherein K_{D} is measured by surface plasmon resonance (Biacore) analysis.

A VH domain, or one or more CDRs thereof, described herein can be linked to a constant domain for forming a heavy chain, e.g., a full length heavy chain. Similarly, a VL domain, or one or more CDRs thereof, described herein can be linked to a constant domain for forming a light chain, e.g., a full length light chain. A full length heavy chain (optionally with the exception of the C-terminal lysine (K) or with the exception of the C-terminal glycine and lysine (GK), which can be absent) and full length light chain may combine to form a full length antibody.

A VH domain described herein can be fused to the constant domain of a human IgG, e.g., IgG1, IgG2, IgG3 or IgG4, which are either naturally-occurring or modified, e.g., as further described herein. For example, a VH domain can comprise the amino acid sequence of any VH domain described herein fused to a human IgG, e.g., an IgG1, constant region, such as the following wild-type human IgG1 constant domain amino acid sequence:

(SEQ ID NO: 40)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

or that of an allotypic variant of SEQ ID NO: 40 and have the following amino acid sequences:

(SEQ ID NO: 41; allotype specific amino acid
residues are in bold and underlined)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

A VH domain of an anti-integrin αv heterodimer antibody can comprise the amino acid sequence of any VH domain described herein fused to an effectorless constant region, e.g., the following effectorless human IgG1 constant domain amino acid sequences:

(SEQ ID NO: 42; “IgG1.1f,” comprising
substitutions L234A, L235E, G237A, A330S and
P331S, which are underlined)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPEAEGAPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPSSIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
or
(SEQ ID NO: 43; “IgG1.3f”, comprising
substitutions L234A, L235E and G237A, which are
underlined)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPEAEGAPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

For example, an allotypic variant of IgG1 comprises K97R, D239E, and/or L241M (underlined and bolded above) as numbered in SEQ ID NOs: 40. In some aspects, the constant region of an anti-integrin αv heterodimer (e.g., integrin-αvβ1) antibody can further comprises one or more mutations or substitutions at amino acids L117, A118, G120, A213, and P214 (underlined above) as numbered in SEQ ID NO: 41, 42, and 43, or L234, A235, G237, A330 and P331, per EU numbering. In some aspects, the constant region of an anti-integrin αv heterodimer (e.g., integrin-αvβ1) antibody comprises one or more mutations or substitutions at amino acids L117A, A118E, G120A, A213S, and P214S of SEQ ID NO: 40, or L234A, L235E, G237A, A330S and P331S, per EU numbering. The constant region of an anti-integrin αv heterodimer (e.g., integrin-αvβ1) antibody may also comprise one or more mutations or substitutions L117A, A118E and G120A of SEQ ID NO: 40, or L234A, L235E and G237A, per EU numbering

Alternatively, a VH domain of an anti-integrin αv heterodimer (e.g., integrin-αvβ1) antibody can comprise the amino acid sequence of any VH domain described herein fused to a human IgG4 constant region, e.g., the following human IgG4 amino acid sequence or variants thereof:

(SEQ ID NO: 44, comprising S228P)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A VL domain described herein can be fused to the constant domain of a human Kappa or Lambda light chain. For example, a VL domain of an anti-integrin αv heterodimer (e.g., integrin-αvβ1) antibody can comprise the amino acid sequence of any VL domain described herein fused to the following human IgG1 kappa light chain amino acid sequence: RTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 45)

In some aspects, the heavy chain constant region comprises a lysine or another amino acid at the C-terminus, e.g., it comprises the following last amino acids: LSPGK (SEQ ID NO: 46) in the heavy chain. In some aspects, the heavy chain constant region is lacking one or more amino acids at the C-terminus, and has, e.g., the C-terminal sequence LSPG (SEQ ID NO: 47) or LSP (SEQ ID NO: 48).

Anti-integrin αv heterodimer (e.g., integrin-αvβ1) antibodies can comprise a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the anti-integrin αv heterodimer (e.g., integrin-αvβ1) antibodies described herein, or conservative modifications thereof, and wherein the antibodies retain the desired functional properties of the anti-integrin αv heterodimer (e.g., integrin-αvβ1) antibodies described herein.

Conservative amino acid substitutions can be made in portions of the antibodies other than, or in addition to, the CDRs. For example, conservative amino acid modifications can be made in a framework region or in the Fc region. A variable region or a heavy or light chain can comprise 1, 2, 3, 4, 5, 1-2, 1-3, 1-4, 1-5, 1-10, 1-15, 1-20, 1-25, or 1-50 conservative amino acid substitutions relative to the anti-integrin αv heterodimer (e.g., integrin-αvβ1) antibody sequences provided herein. In some aspects, an anti-integrin αv heterodimer (e.g., integrin-αvβ1) antibody comprises a combination of conservative and non-conservative amino acid modification.

Also provided are engineered and modified antibodies that can be prepared using an antibody having one or more of the VH and/or VL sequences disclosed herein as starting material to engineer a modified antibody, which modified antibody can have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally-occurring antibodies by constructing expression vectors that include CDR sequences from the specific naturally-occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. Sci. U.S.A. 86: 10029-10033; U.S. Pat. No. 5,225,539 to Winter, and U.S. Pat. Nos. 5,530,101; 5,585,089; 5,693,762 and 6,180,370 to Queen et al.).

Accordingly, some aspects described herein pertain to an isolated monoclonal antibody, or antigen-binding portion thereof, comprising a heavy chain variable region comprising CDR1, CDR2, and CDR3 sequences described herein, and a light chain variable region comprising CDR1, CDR2, and CDR3 sequences described herein. Thus, such antibodies contain the VH and VL CDR sequences of monoclonal antibodies described herein yet can contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops”/. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

In some aspects, the framework sequences for use in the anti-integrin αv heterodimer (e.g., integrin-αvβ1) antibodies described herein are those that are structurally similar to the framework sequences used by the anti-integrin αv heterodimer (e.g., integrin-αvβ1) antibodies described herein. The VH CDR1, CDR2 and CDR3 sequences, and the VL CDR1, CDR2 and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see, e.g., U.S. Pat. Nos. 5,530,101; 5,585,089; 5,693,762; and 6,180,370 to Queen et al.).

Engineered anti-integrin αv heterodimer (e.g., integrin-αvβ1) antibodies described herein include those in which modifications have been made to framework residues within VH and/or VL, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed. Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Another type of variable region modification is to mutate amino acid residues within the VH and/or VL CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. In some aspects, conservative modifications (as discussed above) are introduced. The mutations can be amino acid substitutions, additions or deletions. Moreover, typically no more than one, two, three, four or five residues within a CDR region are altered.

Methionine residues in CDRs of antibodies can be oxidized, resulting in potential chemical degradation and consequent reduction in potency of the antibody. Accordingly, also provided are anti-integrin αv heterodimer (e.g., integrin-αvβ1) antibodies which have one or more methionine residues in the heavy and/or light chain CDRs replaced with amino acid residues which do not undergo oxidative degradation.

Similarly, deamidation sites can be removed from anti-integrin αv heterodimer (e.g., integrin-αvβ1) antibodies, particularly in the CDRs.

Anti-integrin αv heterodimer (e.g., integrin-αvβ1) variable regions described herein can be linked (e.g., covalently linked or fused) to an Fc, e.g., an IgG1, IgG2, IgG3 or IgG4 Fc, which can be of any allotype or isoallotype, e.g., for IgG1: G1m, G1m1(a), G1m2(x), G1m3(f), G1m17(z); for IgG2: G2m, G2m23(n); for IgG3: G3m, G3m21(g1), G3m28(g5), G3m11(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1:1).

Generally, variable regions described herein can be linked to an Fc comprising one or more modification, typically to alter one or more functional properties of the antibody, such as serum half-life, complement fixation, Fc receptor binding, antigen-dependent cellular cytotoxicity, and/or antibody-dependent cellular phagocytosis. Furthermore, an antibody described herein can be chemically modified (e.g., one or more chemical moieties can be attached to the antibody) or be modified to alter its glycosylation, to alter one or more functional properties of the antibody. Each of these aspects is described in further detail below. The numbering of residues in the Fc region is that of the EU index of Kabat.

The Fc region encompasses domains derived from the constant region of an immunoglobulin, including a fragment, analog, variant, mutant or derivative of the constant region. Suitable immunoglobulins include IgG1, IgG2, IgG3, IgG4, and other classes such as IgA, IgD, IgE and IgM, The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

Ig molecules interact with multiple classes of cellular receptors. For example, IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains. The serum half-life of an antibody is influenced by the ability of that antibody to bind to an Fc receptor (FcR).

In some aspects, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity,

Generally, variants of the constant region or portions thereof, e.g., CH1, CL, hinge, CH2 or CH3 domains can comprise 1, 2, 3, 4, 5, 6, 7, 8, 9, 10 or more mutations, and/or at most 10, 9, 8, 7, 6, 5, 4, 3, 2 or 1 mutation, or 1-10 or 1-5 mutations, or comprise an amino acid sequence that is at least about 75%, 80%, 85%, 90%, 95%, 96%, 97%, 98%, or 99% identical to that of the corresponding wild-type region or domain (CH1, CL, hinge, CH2, or CH3 domain, respectively), provided that the heavy chain constant region comprising the specific variant retains the necessary biological activity.

For example, one can make modifications in the Fc region in order to generate an Fc variant that (a) mediates increased or decreased antibody-dependent cell-mediated cytotoxicity (ADCC) and/or antibody-dependent cellular phagocytosis (ADCP), (b) mediates increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region can include two, three, four, five, etc. substitutions therein, e.g., of the specific Fc region positions identified herein.

A variant Fc region can also comprise a sequence alteration wherein amino acids involved in disulfide bond formation are removed or replaced with other amino acids. Such removal can avoid reaction with other cysteine-containing proteins present in the host cell used to produce the anti-integrin αv heterodimer (e.g., integrin-αvβ1) antibodies described herein. Even when cysteine residues are removed, single chain Fc domains can still form a dimeric Fc domain that is held together non-covalently. In some aspects, the Fc region can be modified to make it more compatible with a selected host cell. For example, one can remove the PA sequence near the N-terminus of a typical native Fc region, which can be recognized by a digestive enzyme in E. coli such as proline iminopeptidase. In some aspects, one or more glycosylation sites within the Fc domain can be removed. Residues that are typically glycosylated (e.g., asparagine) can confer cytolytic response. Such residues can be deleted or substituted with unglycosylated residues (e.g., alanine). In some aspects, sites involved in interaction with complement, such as the C1q binding site, can be removed from the Fc region. For example, one can delete or substitute the EKK sequence of human IgG1. In some aspects, sites that affect binding to Fc receptors can be removed, preferably sites other than salvage receptor binding sites. In some aspects, an Fc region can be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631, WO 96/32478 and WO07/041635.

In some aspects, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In some aspects, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some aspects, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In another example, the Fc region can be modified to enhance affinity for an Fcγ and increase macrophage-mediated phagocytosis. See, e.g., Richard et al., Mo. Cancer. Ther. 7(8):2517-27 (2008), which is incorporated by reference herein in its entirety. In certain aspects, the Fc region can be modified to increase affinity for FcγRIIa relative to inhibitory FcγRIIb. One particular point mutation, G236A (whose numbering is according to the EU index), has been identified as having increased affinity for FcγRIIa relative to inhibitory FcγRIIb. This increased affinity for FcRIIa correlated with increased macrophage-mediated phagocytosis, relative to native IgG1. In some aspects, the Fc region of the anti-integrin αv heterodimer (e.g., integrin-αvβ1) antibody comprises one or more mutation or combination of mutations selected from G236A, I332E, S239/I332E, I332E/G236A, and S239D/I332E/G236A. Other modifications to the Fc region can increase antibody dependent cellular cytotoxicity (ADCC), e.g., by increasing affinity for activating receptors such as FcγRI and/or FcγRIIIa. For example, the G236A substitution, and combination of the G236A substitution with modifications that improve affinity for activating receptors (e.g., FcγRI and/or FcγRIIIa), for example including but not limited to substitutions at 332 and 239, provide substantially improved ADCC relative to the parent WT antibody. See U.S. Pat. No. 9,040,041, which is incorporated by reference herein in its entirety.

In another example, the Fc region can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Optionally, the Fc region can comprise a non-naturally-occurring amino acid residue at additional and/or alternative positions (see, e.g., U.S. Pat. Nos. 5,624,821; 6,277,375; 6,737,056; 6,194,551; 7,317,091; 8,101,720; 9,040,041; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217; WO 05/092925; and WO 06/0201 14).

The affinities and binding properties of an Fc region for its ligand can be determined by a variety of in vitro assay methods (biochemical or immunological based assays) known in the art including but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In some aspects, the antibody is modified to increase its biological half-life. Various approaches are possible. For example, this can be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

In some aspects, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus a hybrid variant IgG antibody can be constructed that comprises one or more substitutions, e.g., 274Q, 276K, 300F, 339T, 356E, 358M, 384S, 392N, 397M, 4221, 435R, and 436F. In some aspects described herein, an IgG1/IgG2 hybrid variant can be constructed by substituting IgG2 positions in the CH2 and/or CH3 region with amino acids from IgG1 at positions where the two isotypes differ. Thus a hybrid variant IgG antibody can be constructed that comprises one or more substitutions, e.g., one or more of the following amino acid substitutions: 233E, 234L, 235L, −236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/I332E, S239D/I332E/A330L, L235V/F243L/R292P/Y300L/P396L, and M428L/N434S.

Specific mutations at positions 234, 235, 236, 239, 267, 268, 293, 295, 324, 327, 328, 330, and 332 were shown to improve binding to FcγRIIa and/or reduce binding to FcγRIIb, resulting in enhanced ADCC and/or ADCP activity (Richards et al., Mol. Cancer Ther. 7(8):2517-2527; U.S. Pat. No. 9,040,041). In particular, Fc variants that selectively improve binding to one or more human activating receptors relative to FcγRIIb, or selectively improve binding to FcγRIIb relative to one or more activating receptors, may comprise a substitution selected from the group consisting of 234G, 234I, 235D, 235E, 235I, 235Y, 236A, 236S, 239D, 267D, 267E, 267Q, 268D, 268E, 293R, 295E, 324G, 324I, 327H, 328A, 328F, 328I, 330I, 330L, 330Y, 332D, and 332E. Additional substitutions that may also be combined include other substitutions that modulate FcγR affinity and complement activity, including but not limited to 298A, 298T, 326A, 326D, 326E, 326W, 326Y, 333A, 333S, 334L, and 334A (U.S. Pat. No. 6,737,056; Shields et al, Journal of Biological Chemistry, 2001, 276(9):6591-6604; U.S. Pat. No. 6,528,624; Idusogie et al., 2001, J. Immunology 166:2571-2572). Preferred variants that may be particularly useful to combine with other Fc variants include those that comprise the substitutions 298A, 326A, 333A, and 334A. Additional substitutions that may be combined with the FcγR selective variants include 247L, 255L, 270E, 392T, 396L, and 421K (U.S. Ser. No. 10/754,922; U.S. Ser. No. 10/902,588); and 280H, 280Q, and 280Y (U.S. Ser. No. 10/370,749).

When using an IgG4 constant domain, it can include the substitution S228P, which mimics the hinge sequence in IgG1 and thereby stabilizes IgG4 molecules.

II.B. Bispecific and Multispecific Antibodies

Some aspects of the present disclosure are directed to bispecific or multispecific antibodies that specifically bind an integrin-αv heterodimer (e.g., integrin-αvβ1). In some aspects, the bispecific or multispecific antibody binds an integrin-αv heterodimer (e.g., integrin-αvβ1) and a second antigen. In some aspects, the second antigen is not an integrin-αv heterodimer (e.g., integrin-αvβ1). In some aspects, the bispecific or multispecific antibody binds a first epitope on an integrin-αv heterodimer (e.g., integrin-αvβ1) and a second epitope on an integrin-αv heterodimer (e.g., integrin-αvβ1). In some aspects, the first epitope and the second epitope are not overlapping.

Any anti-integrin αv heterodimer (e.g., integrin-αvβ1) antibodies described herein can be used for forming bispecific or multispecific antibodies. An anti-integrin αv heterodimer (e.g., integrin-αvβ1) antibody, or antigen-binding portions thereof, can be derivatized or linked to another functional molecule, e.g., another peptide or protein (e.g., another antibody or ligand for a receptor) to generate a bispecific or multispecific molecule that binds to at least two different binding sites or target molecules. In some aspects, an anti-integrin αv heterodimer (e.g., integrin-αvβ1) antibody is linked to an antibody or scFv that binds specifically to any protein that can be used as potential targets for combination treatments, such as the proteins described herein (e.g., antibodies to PD-1, PD-L1, CTLA-4, LAG3, TIGIT, TIM3, NKG2a, OX40, ICOS, CD137, KIR, TGFβ, IL-10, IL-2, IL-8, B7-H4, Fas ligand, CXCR4, mesothelin, CD27, CD96, VISTA, or GITR, or pegylated IL-2 or pegylated IL-10). The antibody described herein can in fact be derived or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for integrin αv heterodimer (e.g., integrin-αvβ1) and a second binding specificity for a second target epitope. In some aspects described herein in which the bispecific molecule is multispecific, the molecule can further include a third binding specificity.

In some aspects, the bispecific molecules described herein comprise as a binding specificity at least one antibody, or an antibody fragment thereof, including, e.g., an Fab, Fab′, F(ab′)2, Fv, or a single chain Fv (scFv). The antibody can also be a light chain or heavy chain dimer, or any minimal fragment thereof such as a Fv or a single chain construct as described in Ladner et al. U.S. Pat. No. 4,946,778.

While human monoclonal antibodies are preferred, other antibodies which can be employed in the bispecific molecules described herein are murine, chimeric and humanized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see, e.g., Karpovsky et al. (1984) J. Exp. Med. 160: 1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Some conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In some aspects, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, mAb×(scFv)₂, Fab×F(ab′)₂ or ligand x Fab fusion protein. A bispecific antibody can comprise an antibody comprising an scFv at the C-terminus of each heavy chain. A bispecific molecule described herein can be a single chain molecule comprising one single chain antibody and a binding determinant, or a single chain bispecific molecule comprising two binding determinants. Bispecific molecules can comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In some aspects, the bispecific antibody or an antigen-binding portion thereof comprises at least a first paratope and a second paratope, wherein the first paratope binds a first epitope on an integrin αv heterodimer (e.g., integrin-αvβ1) heterodimer. In some aspects, the second paratope binds a second epitope on the integrin αv heterodimer (e.g., integrin-αvβ1) heterodimer. In some aspects, the first epitope and the second epitope are not the same. In some aspects, the first epitope and the second epitope are not overlapping.

In some aspects, bispecific antibody or antigen-binding portion thereof comprises a first heavy chain, a first light chain, a second heavy chain, and second light chain. In some aspects, the first heavy chain and the second heavy chain are different. In some aspects, the first light chain and the second light chain are different. In some aspects, the first heavy chain and the second heavy chain are different, and the first light chain and the second light chain are the same. In some aspects, the first heavy chain and the second heavy chain are the same, and the first light chain and the second light chain are different.

The first heavy chain, the second heavy chain, the first light chain, and the second light chain can comprise any heavy chain or light chain disclosed herein. In some aspects, the first heavy chain comprises a first variable heavy region (“VH1”), comprising a variable heavy complementarity determining region (VH1-CDR) 1, a VH1-CDR2, and a VH1-CDR3; wherein the VH1-CDR3 comprises an amino acid sequence selected from SEQ ID NOs: 5, 15, and 25. In some aspects, the VH1-CDR2 comprises an amino acid sequence selected from SEQ ID NOs: 4, 14, and 24. In some aspects, the VH1-CDR1 comprises an amino acid sequence selected from SEQ ID NOs: 3, 13, and 23. In some aspects, the first light chain comprises a first variable light region (“VL1”), comprising a VL1-CDR1, a VL1-CDR2, and a VL1-CDR3; wherein the VL1-CDR3 comprises an amino acid sequence selected from SEQ ID NOs: 10, 20, and 30. In some aspects, the VL1-CDR2 comprises an amino acid sequence selected from SEQ ID NOs: 9, 19, and 29. In some aspects, the VL1-CDR1 comprises an amino acid sequence selected from SEQ ID NOs: 8, 18, and 28.

In certain aspects, the bispecific antibody or antigen-binding portion thereof comprises a VH1-CDR1 comprising the amino acid sequence set forth in SEQ ID NO: 3; a VH1-CDR2 comprising the amino acid sequence set forth in SEQ ID NO: 4; a VH1-CDR3 comprising the amino acid sequence set forth in SEQ ID NO: 5; a VL1-CDR1 comprising the amino acid sequence set forth in SEQ ID NO: 18; a VL1-CDR2 comprising the amino acid sequence set forth in SEQ ID NO: 19; and a VL1-CDR3 comprising the amino acid sequence set forth in SEQ ID NO: 20.

In certain aspects, the bispecific antibody or antigen-binding portion thereof comprises a VH1-CDR1 comprising the amino acid sequence set forth in SEQ ID NO: 13; a VH1-CDR2 comprising the amino acid sequence set forth in SEQ ID NO: 14; a VH1-CDR3 comprising the amino acid sequence set forth in SEQ ID NO: 15; a VL1-CDR1 comprising the amino acid sequence set forth in SEQ ID NO: 18; a VL1-CDR2 comprising the amino acid sequence set forth in SEQ ID NO: 19; and a VL1-CDR3 comprising the amino acid sequence set forth in SEQ ID NO: 20.

In certain aspects, the bispecific antibody or antigen-binding portion thereof comprises a VH1-CDR1 comprising the amino acid sequence set forth in SEQ ID NO: 3; a VH1-CDR2 comprising the amino acid sequence set forth in SEQ ID NO: 4; a VH1-CDR3 comprising the amino acid sequence set forth in SEQ ID NO: 5; a VL1-CDR1 comprising the amino acid sequence set forth in SEQ ID NO: 28; a VL1-CDR2 comprising the amino acid sequence set forth in SEQ ID NO: 29; and a VL1-CDR3 comprising the amino acid sequence set forth in SEQ ID NO: 30.

In certain aspects, the bispecific antibody or antigen-binding portion thereof comprises a VH1-CDR1 comprising the amino acid sequence set forth in SEQ ID NO: 23; a VH1-CDR2 comprising the amino acid sequence set forth in SEQ ID NO: 24; a VH1-CDR3 comprising the amino acid sequence set forth in SEQ ID NO: 25; a VL1-CDR1 comprising the amino acid sequence set forth in SEQ ID NO: 28; a VL1-CDR2 comprising the amino acid sequence set forth in SEQ ID NO: 29; and a VL1-CDR3 comprising the amino acid sequence set forth in SEQ ID NO: 30.

In some aspects, the second heavy chain comprises a second variable heavy region (“VH2”), comprising a VH2-CDR1, a VH2-CDR2, and a VH2-CDR3; wherein the VH2-CDR3 comprises an amino acid sequence selected from SEQ ID NOs: 5, 15, and 25. In some aspects, the VH2-CDR2 comprises an amino acid sequence selected from SEQ ID NOs: 4, 14, and 24. In some aspects, the VH2-CDR1 comprises an amino acid sequence selected from SEQ ID NOs: 3, 13, and 23. In some aspects, the second light chain comprises a second variable light region (“VL1”), comprising a VL2-CDR1, a VL2-CDR2, and a VL2-CDR3; wherein the VL2-CDR3 comprises an amino acid sequence selected from SEQ ID NOs: 20 and 30. In some aspects, the VL2-CDR2 comprises an amino acid sequence selected from SEQ ID NOs: 19 and 29. In some aspects, the VH2-CDR1 comprises an amino acid sequence selected from SEQ ID NOs: 18 and 28.

In certain aspects, the bispecific antibody or antigen-binding portion thereof comprises a VH1-CDR1 comprising the amino acid sequence set forth in SEQ ID NO: 3; a VH1-CDR2 comprising the amino acid sequence set forth in SEQ ID NO: 4; a VH1-CDR3 comprising the amino acid sequence set forth in SEQ ID NO: 5; a VL1-CDR1 comprising the amino acid sequence set forth in SEQ ID NO: 18; a VL1-CDR2 comprising the amino acid sequence set forth in SEQ ID NO: 19; a VL1-CDR3 comprising the amino acid sequence set forth in SEQ ID NO: 20; a VH2-CDR1 comprising the amino acid sequence set forth in SEQ ID NO: 13; a VH2-CDR2 comprising the amino acid sequence set forth in SEQ ID NO: 14; a VH2-CDR3 comprising the amino acid sequence set forth in SEQ ID NO: 15; a VL2-CDR1 comprising the amino acid sequence set forth in SEQ ID NO: 18; a VL2-CDR2 comprising the amino acid sequence set forth in SEQ ID NO: 19; and a VL2-CDR3 comprising the amino acid sequence set forth in SEQ ID NO: 20.

In certain aspects, the bispecific antibody or antigen-binding portion thereof comprises a VH1-CDR1 comprising the amino acid sequence set forth in SEQ ID NO: 3; a VH1-CDR2 comprising the amino acid sequence set forth in SEQ ID NO: 4; a VH1-CDR3 comprising the amino acid sequence set forth in SEQ ID NO: 5; a VL1-CDR1 comprising the amino acid sequence set forth in SEQ ID NO: 28; a VL1-CDR2 comprising the amino acid sequence set forth in SEQ ID NO: 29; a VL1-CDR3 comprising the amino acid sequence set forth in SEQ ID NO: 30; a VH2-CDR1 comprising the amino acid sequence set forth in SEQ ID NO: 23; a VH2-CDR2 comprising the amino acid sequence set forth in SEQ ID NO: 24; a VH2-CDR3 comprising the amino acid sequence set forth in SEQ ID NO: 25; a VL2-CDR1 comprising the amino acid sequence set forth in SEQ ID NO: 28; a VL2-CDR2 comprising the amino acid sequence set forth in SEQ ID NO: 29; and a VL2-CDR3 comprising the amino acid sequence set forth in SEQ ID NO: 30.

In some aspects, the bispecific antibody or antigen-binding portion thereof comprises a first variable heavy region (VH1) and a first variable light region (VL1), wherein the VH1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22. In some aspects, the VL1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

In some aspects, the bispecific antibody or antigen-binding portion thereof comprises a second variable heavy region (VH2) and a second variable light region (VL2), wherein the VH2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22. In some aspects, the VL2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

In some aspects, (a) the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; (b) the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17; (c) the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12; and (d) the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the VH1 comprises the amino acid sequence set forth in SEQ ID NO: 2; the VL1 comprises the amino acid sequence set forth in SEQ ID NO: 17; the VH2 comprises the amino acid sequence set forth in SEQ ID NO: 12; and the VL2 comprises the amino acid sequence set forth in SEQ ID NO: 17.

In some aspects, (a) the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; (b) the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27; (c) the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 22; and (d) the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27. In some aspects, the VH1 comprises the amino acid sequence set forth in SEQ ID NO: 2; the VL1 comprises the amino acid sequence set forth in SEQ ID NO: 27; the VH2 comprises the amino acid sequence set forth in SEQ ID NO: 22; and the VL2 comprises the amino acid sequence set forth in SEQ ID NO: 27.

In some aspects, the bispecific antibody or antigen-binding portion thereof comprises a first heavy chain (H1) and a first light chain (L1), wherein the H1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37. In some aspects, the L1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

In some aspects, the bispecific antibody or antigen-binding portion thereof comprises a second heavy chain (H2) and a second light chain (L2), wherein the H2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37. In some aspects, the L2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

In some aspects, the first heavy chain is associated with the second heavy chain. In some aspects, the first heavy chain is associated with the second heavy chain by one or more covalent bond. In some aspects, each of the first heavy chain and the second heavy chain comprises an IgG constant region or an IgG constant region comprising one or more amino acid substitutions. In some aspects, the one or more amino acid substitutions promotes heterodimerization of the first heavy chain and the second heavy chain. Any amino acid substitutions can be used to facilitate heterodimerization. In some aspects, the first heavy chain comprises a substitution of one or more amino acids in a constant region of the first heavy chain, creating a knob; wherein the second heavy chain comprises a substitution or antigen-binding portion thereof of one or more amino acids in a constant region of the second heavy chain, creating a hole; wherein the knob of the first heavy chain associates with the hole of the second heavy chain. In some aspects, the first heavy chain comprises a substitution of one or more amino acids in a constant region of the first heavy chain, creating a hole; wherein the second heavy chain comprises a substitution of one or more amino acids in a constant region of the second heavy chain, creating a knob; wherein the hole of the first heavy chain associates with the knob of the second heavy chain.

In some aspects, (a) the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31; (b) the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16; (c) the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 34; and (d) the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16.

In some aspects, the H1 comprises the amino acid sequence set forth in SEQ ID NO: 31; the L1 comprises the amino acid sequence set forth in SEQ ID NO: 16; the H2 comprises the amino acid sequence set forth in SEQ ID NO: 34; and the L2 comprises the amino acid sequence set forth in SEQ ID NO: 16.

In some aspects, (a) the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31; (b) the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26; (c) the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 37; and (d) the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26.

In some aspects, the H1 comprises the amino acid sequence set forth in SEQ ID NO: 31; the L1 comprises the amino acid sequence set forth in SEQ ID NO: 26; the H2 comprises the amino acid sequence set forth in SEQ ID NO: 37; and the L2 comprises the amino acid sequence set forth in SEQ ID NO: 26.

In some aspects, the bispecific antibody inhibits binding of integrin αv heterodimer (e.g., integrin-αvβ1) to LAP-TGFβ1. In some aspects, binding of integrin αv heterodimer (e.g., integrin-αvβ1) to LAP-TGFβ1 is reduced by to less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1% relative to the level of binding of integrin αv heterodimer (e.g., integrin-αvβ1) to LAP-TGFβ1 in the absence of the bispecific antibody.

In some aspects, the bispecific antibody is capable of binding an integrin αv heterodimer selected from αvβ1, αvβ3, αvβ5, αvβ6, αvβ8, and any combination thereof.

In some aspects, the bispecific antibody inhibits cell adhesion. In some aspects, the bispecific antibody inhibits tumor growth and/or metastasis. In some aspects, the bispecific antibody increases progression-free survival. In some aspects, the bispecific antibody increases overall survival.

II.C. Immunoconjugates, Antibody Derivatives and Diagnostics

Anti-integrin αv antibodies described herein can be used for diagnostic purposes, including sample testing and in vivo imaging, and for this purpose the antibody (or binding fragment thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing.

The detectable labels that can be linked to any anti-integrin αv heterodimer (e.g., integrin-αvβ1) antibody described herein can be any of the various types used currently in the field of in vitro diagnostics, including particulate labels including metal sols such as colloidal gold, isotopes such as I¹²⁵ or Tc⁹⁹ presented for instance with a peptidic chelating agent of the N₂S₂, N₃S or N₄ type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-STAR® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

In some aspects, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see e.g., Senter, P. D., Curr. Opin. Chem. Biol. 13 (2009) 235-244; WO 2009/059278; WO 95/17886).

Depending on the biochemical nature of the moiety and the antibody, different conjugation strategies can be employed. In case the moiety is naturally-occurring or recombinant of between 50 to 500 amino acids, there are standard procedures in text books describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some aspects, the reaction of a maleinimido moiety with a cysteine residue within the antibody or the moiety is used. This is an especially suited coupling chemistry in case e.g., a Fab or Fab′-fragment of an antibody is used. Alternatively, in some aspects, coupling to the C-terminal end of the antibody or moiety is performed. C-terminal modification of a protein, e.g., of a Fab-fragment, can be performed as described (Sunbul, M. and Yin, J., Org. Biomol. Chem. 7 (2009) 3361-3371).

In general, site-specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al. Chem. Biol. 15 (2008) 128-136; and Protease-catalyzed formation of C N bonds is used by Bordusa, F., Highlights in Bioorganic Chemistry (2004) 389-403). Site-specific reaction and covalent coupling can also be achieved by the selective reaction of terminal amino acids with appropriate modifying reagents.

The reactivity of an N-terminal cysteine with benzonitrils (see Ren, H. et al., Angew. Chem. Int. Ed. Engl. 48 (2009) 9658-9662) can be used to achieve a site-specific covalent coupling.

Native chemical ligation can also rely on C-terminal cysteine residues (Taylor, E. Vogel; Imperiali, B, Nucleic Acids and Molecular Biology (2009), 22 (Protein Engineering), 65-96).

U.S. Pat. No. 6,437,095 B1 describes a conjugation method which is based on the faster reaction of a cysteine within a stretch of negatively charged amino acids with a cysteine located in a stretch of positively charged amino acids.

The moiety can also be a synthetic peptide or peptide mimic. In case a polypeptide is chemically synthesized, amino acids with orthogonal chemical reactivity can be incorporated during such synthesis (see e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

In some aspects the moiety attached to an anti-integrin αv heterodimer (e.g., integrin-αvβ1) antibody is selected from the group consisting of a binding moiety, a labeling moiety, and a biologically active moiety.

Anti-integrin αv heterodimer (e.g., integrin-αvβ1) antibodies described herein can also be conjugated to a therapeutic agent to form an immunoconjugate such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. In some aspects, the linker is a peptidyl linker such as Val-Cit, Ala-Val, Val-Ala-Val, Lys-Lys, Pro-Val-Gly-Val-Val (SEQ ID NO: 108), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295.

In some aspects, the therapeutic agent is selected from the group consisting of a cytotoxin, a non-cytotoxic drug, a radioactive agent, a second antibody, an enzyme, an anti-neoplastic agent, and any combination thereof.

In some aspects, the immunoconjugate comprises an anti-integrin αv heterodimer (e.g., integrin-αvβ1) antibody and a cytotoxin. The cytotoxin can be selected from any cytotoxin known in the art. In some aspects, the cytotoxin is selected from the group consisting of dolastatin, monomethyl auristatin E (MMAE), maytansine, duocarmycin, calicheamicin, pyrrolobenzodiazepine, duocarmycin, centanamycin, SN38, doxorubicin, a derivative thereof, a synthetic analog thereof, and any combination thereof. In certain aspects, the immunoconjugate comprises an anti-integrin αv heterodimer (e.g., integrin-αvβ1) antibody and Cytotoxin A. In other aspects, the immunoconjugate comprises an anti-integrin αv heterodimer (e.g., integrin-αvβ1) antibody and a non-cytotoxic drug.

In some aspects, the immunoconjugate comprises an anti-integrin αv heterodimer (e.g., integrin-αvβ1) antibody and a radioactive agent. In some aspects, the radioactive agent is a radionucleotide. In certain aspects, the radioactive agent comprises radioactive iodine. In particular aspects, the radioactive agent comprises 131-iodine. In other aspects, the radioactive agent comprises the radioactive isotope Yttrium-90.

In some aspects, the immunoconjugate comprises an anti-integrin αv heterodimer (e.g., integrin-αvβ1) antibody and a second antibody. The second antibody can be any antibody described in the present disclosure, including, but not limited to, an antibody that specifically binds a protein selected from the group consisting of PD-1, PD-L1, CTLA-4, LAG3, TIGIT, TIM3, NKG2a, OX40, ICOS, CD137, KIR, TGFβ, IL-10, IL-2, IL-8, B7-H4, Fas ligand, CXCR4, mesothelin, VISTA, CD96, CD27, GITR, and any combination thereof. In one aspect, the immunoconjugate comprises an anti-integrin αv heterodimer (e.g., integrin-αvβ1) antibody and an anti-PD-1 antibody. In another aspect, the immunoconjugate comprises an anti-integrin αv heterodimer (e.g., integrin-αvβ1) antibody and nivolumab.

In one aspect, the immunoconjugate comprises an anti-integrin αv heterodimer (e.g., integrin-αvβ1) antibody and a pegylated IL-2 or pegylated IL-10.

In certain aspects, the immunoconjugate comprises an anti-integrin αv heterodimer (e.g., integrin-αvβ1) antibody and an enzyme. In some aspects, the enzyme comprises glucose oxidase. In some aspects, the enzyme comprises a peroxidase. In some aspects, the enzyme comprises myeloperoxidase. In some aspects, the enzyme comprises glucose oxidase. In some aspects, the enzyme comprises horseradish peroxidase.

In certain aspects, the immunoconjugate comprises an anti-integrin αv heterodimer (e.g., integrin-αvβ1) antibody and an anti-neoplastic agent. The anti-neoplastic agent can be any such agent known in the art. In some aspects, the anti-neoplastic agent is epirubicin. In some aspects, the anti-neoplastic agent is a super antigen. In certain aspects, the super antigen is staphylococcal enterotoxin A (SEA/E-120; estafenatox).

Anti-integrin αv heterodimer (e.g., integrin-αvβ1) antibodies, e.g., those described herein, can also be used for detecting integrin αv heterodimer (e.g., integrin-αvβ1). The antibodies can be used, e.g., in an ELISA assay or in flow cytometry.

Other uses for anti-integrin αv heterodimer (e.g., integrin-αvβ1) antibodies, e.g., as monotherapy or combination therapy, are provided elsewhere herein, e.g., in the section pertaining to combination treatments.

II.D. Nucleic Acid Molecules

Another aspect described herein pertains to nucleic acid molecules that encode the anti-integrin αv heterodimer (e.g., integrin-αvβ1) antibodies described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Some nucleic acids molecules described herein are those encoding the VH and VL sequences of the anti-integrin αv heterodimer antibodies.

The nucleic acid molecules described herein may be modified to delete specific sequences, e.g., restriction enzyme recognition sequences, or to optimize codons.

A method for making anti-integrin αv heterodimer (e.g., integrin-αvβ1) antibodies disclosed herein can comprise expressing the heavy chain and the light chains in a cell line comprising the nucleotide sequences encoding the heavy and light chains with a signal peptide. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2, and/or CH3). The sequences of human heavy chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see, e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see, e.g., Bird et al., (1988) Science 242:423-426; Huston et al., (1988) Proc. Natl. Acad. Sci. USA 85:5879-5883; McCafferty et al., (1990) Nature 348:552-554).

Also provided herein are nucleic acid molecules with conservative substitutions (i.e., substitutions that do not alter the resulting amino acid sequence upon translation of nucleic acid molecule), e.g., for codon optimization.

II.E. Pharmaceutical Compositions

Further provided are compositions, e.g., pharmaceutical compositions, containing one or a combination of anti-integrin-αv heterodimer (e.g., integrin-αvβ1) antibodies or combination with antibodies to other targets, or antigen-binding portion(s) thereof, described herein, formulated together with a pharmaceutically acceptable carrier. Such compositions can include one or a combination of (e.g., two or more different) antibodies, or immunoconjugates or bispecific molecules described herein. For example, a pharmaceutical composition described herein can comprise a combination of antibodies (inclusing immunoconjugates or bispecifics) that bind to different epitopes on the target antigen or that have complementary activities.

In some aspects, a composition comprises an anti-integrin-αv heterodimer (e.g., integrin-αvβ1) antibody at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

Pharmaceutical compositions described herein also can be administered in combination therapy, i.e., combined with other agents. For example, the combination therapy can include an anti-integrin-αv heterodimer (e.g., integrin-αvβ1) antibody described herein combined with at least one other anti-cancer and/or immunomodulating, e.g., T-cell stimulating (e.g., activating) agent. Examples of therapeutic agents that can be used in combination therapy are described in greater detail below in the section on uses of the anti-integrin-αv heterodimer (e.g., integrin-αvβ1) antibodies described herein.

In some aspects, the anti-integrin-αv heterodimer (e.g., integrin-αvβ1) antibody is combined with at least one other agent selected from chemotherapy drugs, small molecule drugs and antibodies that stimulate the immune response to a given cancer. In some instances, the anti-integrin-αv heterodimer (e.g., integrin-αvβ1) antibody is combined with, for example, one or more of an anti-CTLA-4 antibody, an anti-PD-1 antibody, an anti-PD-L1 antibody, an anti-OX40 (also known as CD134, TNFRSF4, ACT35 and/or TXGP1L) antibody (e.g., BMS986178, or MDX-1803), an anti-CD137 antibody, an anti-LAG-3 antibody, an anti-GITR antibody, an anti-KIR antibody, an anti-TGFβ antibody, an anti-IL-10 antibody, a long-acting IL-10 molecule (e.g. IL-10-Fc fusion, or Pegylated IL-10, such as AM0010 of ARMO BioSciences), a long-acting IL-2 (e.g., Pegylated IL-2 molecules, such as NKTR-214 of Nektar; see U.S. Pat. No. 8,252,275, WO12/065086 and WO15/125159), an anti-VISTA antibody, an anti-CD96 antibody, an anti-IL-8 antibody, an anti-B7-H4, an anti-Fas ligand antibody, an anti-CXCR4 antibody, an anti-mesothelin antibody, an anti-CD27 antibody, an anti-MICA/B antibody, or any combination thereof.

In other aspects, the anti-integrin-αv heterodimer (e.g., integrin-αvβ1) antibody is formulated with a second antibody. In some aspects, the second antibody specifically binds a protein selected from the group consisting of PD-1, PD-L1, CTLA-4, LAG3, TIGIT, TIM3, NKG2a, OX40, ICOS, CD137, KIR, TGFβ, IL-10, IL-2, VISTA, CD96, IL-8, B7-H4, Fas ligand, CXCR4, mesothelin, CD27, GITR, MICA/B, and any combination thereof.

In some aspects, the second antibody is an anti-PD-1 antibody. The anti-PD-1 antibody can be any antibody that binds PD-1 and inhibits the interaction of PD-1 and PD-L1. In some aspects, the anti-PD-1 antibody is any anti-PD-1 antibody disclosed herein. In some aspects, the second antibody is nivolumab. In some aspects, the second antibody is pembrolizumab.

In some aspects, the second antibody is an anti-PD-L1 antibody. The anti-PD-L1 antibody can be any antibody that binds PD-L1 and inhibits the interaction of PD-1 and PD-L1. In some aspects, the anti-PD-L1 antibody is any anti-PD-L1 antibody disclosed herein. In some aspects, the second antibody is atezolizumab. In some aspects, the second antibody is durvalumab. In some aspects, the second antibody is avelumab.

In some aspects, the second antibody is an anti-CTLA-4 antibody. The anti-CTLA-4 antibody can be any antibody that binds CTLA-4 and inhibits its activity. In some aspects, the anti-CTLA-4 antibody is any anti-CTLA-4 antibody disclosed herein. In some aspects, the second antibody is tremelimumab. In some aspects, the second antibody is ipilimumab.

In some aspects, the second antibody is an anti-LAG3 antibody. The anti-LAG3 antibody can be any antibody that binds LAG-3 and inhibits its activity. In some aspects, the anti-LAG3 antibody is any anti-LAG3 antibody disclosed herein. In some aspects, the second antibody is 25F7.

In some aspects, the second antibody is an anti-CD137 antibody. The anti-CD137 antibody can be any antibody that binds CD137 and inhibits its activity. In some aspects, the anti-CD137 antibody is any anti-CD137 antibody disclosed herein. In some aspects, the second antibody is urelumab.

In some aspects, the second antibody is an anti-KIR antibody. The anti-KIR antibody can be any antibody that binds KIR and inhibits its activity. In some aspects, the anti-KIR antibody is any anti-KIR antibody disclosed herein. In some aspects, the second antibody is lirilumab.

In some aspects, the second antibody is an anti-GITR antibody. The anti-GITR antibody can be any antibody that binds GITR and inhibits its activity. In some aspects, the anti-GITR antibody is any anti-GITR antibody disclosed herein. In some aspects, the second antibody is MK4166. In some aspects, the second antibody is TRX518.

In some aspects, the second antibody is an anti-CD96 antibody.

In some aspects, the second antibody is an anti-TIM3 antibody.

In some aspects, the second antibody is an anti-VISTA antibody.

In some aspects, the second antibody is an anti-NKG2a antibody.

In some aspects, the second antibody is an anti-ICOS antibody.

In some aspects, the second antibody is an anti-OX40 antibody.

In some aspects, the second antibody is an anti-IL8 antibody, such as HuMax®-IL8 (BMS-986253).

In some aspects, the anti-integrin-αv heterodimer (e.g., integrin-αvβ1) antibody is formulated with a long-acting IL-10 molecule. In some aspects, the anti-integrin-αv heterodimer (e.g., integrin-αvβ1) antibody is formulated with IL-10-Fc fusion molecule. In some aspects, the anti-integrin-αv heterodimer (e.g., integrin-αvβ1) antibody is formulated with Pegylated IL-10, such as AM0010 of ARMO BioSciences.

In some aspects, the anti-integrin-αv heterodimer (e.g., integrin-αvβ1) antibody is formulated with a long-acting IL-2. In some aspects, the anti-integrin-αv heterodimer (e.g., integrin-αvβ1) antibody is formulated with Pegylated IL-2 molecules, such as NKTR-214 of Nektar; see U.S. Pat. No. 8,252,275, WO12/065086 and WO15/125159.

In some aspects, the composition of the disclosure further comprises a bulking agent. A bulking agent can be selected from the group consisting of NaCl, mannitol, glycine, alanine, and any combination thereof. In other aspects, the composition of the disclosure comprises a stabilizing agent. The stabilizing agent can be selected from the group consisting of sucrose, trehalose, raffinose, arginine; or any combination thereof. In other aspects, the composition of the disclosure comprises a surfactant. The surfactant can be selected from the group consisting of polysorbate 80 (PS80), polysorbate 20 (PS20), and any combination thereof. In certain aspects, the composition further comprises a chelating agent. The chelating agent can be selected from the group consisting of diethylenetriaminepentaacetic acid (DTPA), ethylenediaminetetraacetic acid, nitrilotriacetic acid, and any combination thereof.

In other aspects, the composition comprises a third antibody. In some aspects, the third antibody is any antibody disclosed herein.

In one aspect, the composition further comprises NaCl, mannitol, pentetic acid (DTPA), sucrose, PS80, and any combination thereof.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). An option for subcutaneous injection is based on Halozyme Therapeutics' ENHANZE® drug-delivery technology, involving a co-formulation of an Ab with recombinant human hyaluronidase enzyme (rHuPH20) that removes traditional limitations on the volume of biologics and drugs that can be delivered subcutaneously due to the extracellular matrix (U.S. Pat. No. 7,767,429). Depending on the route of administration, the active compound, i.e., antibody, immunoconjugate, or bispecific molecule, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein can also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition can comprise a preservative or can be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-integrin-αv heterodimer (e.g., integrin-αvβ1) antibody described herein can result in a decrease in severity of disease symptoms, an increase in frequency and duration of disease symptom-free periods, or a prevention of impairment or disability due to the disease affliction. In the context of cancer, a therapeutically effective dose can result in increased survival, e.g., overall survival, and/or prevention of further deterioration of physical symptoms associated with cancer. Symptoms of cancer are well-known in the art and include, for example, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like.

A therapeutically effective dose can prevent or delay onset of cancer, such as can be desired when early or preliminary signs of the disease are present. Laboratory tests utilized in the diagnosis of cancer include chemistries (including the measurement of integrin-αv heterodimer (e.g., integrin-αvβ1) levels), hematology, serology and radiology. Accordingly, any clinical or biochemical assay that monitors any of the foregoing can be used to determine whether a particular treatment is a therapeutically effective dose for treating cancer.

A composition described herein can be administered via one or more routes of administration using one or more of a variety of methods known in the art. The route and/or mode of administration will vary depending upon the desired results. Routes of administration for the anti-integrin-αv heterodimer (e.g., integrin-αvβ1) antibodies described herein can include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein could potentially be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in some aspects, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-integrin-αv heterodimer (e.g., integrin-αvβ1) antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference.

In some aspects, the anti-integrin-αv heterodimer (e.g., integrin-αvβ1) antibodies described herein can be formulated to ensure proper distribution in vivo. For example, the blood-brain barrier (BBB) excludes many highly hydrophilic compounds. To ensure that the therapeutic compounds described herein cross the BBB (if desired, e.g., for brain cancers), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233: 134); pl20 (Schreier et al. (1994) J. Biol. Chem. 269:9090); see also K. Keinanen; M. L. Laukkanen (1994) FEBS Lett. 346: 123; J. J. Killion; I. J. Fidler (1994) Immunomethods 4:273.

### III. Methods of the Disclosure

III.A. Uses and Methods

Certain aspects of the present disclosure are directed to method of treating a subject, comprising administering to the subject an anti-integrin-αv heterodimer (e.g., integrin-αvβ1) antibody disclosed herein, a polynucleotide encoding the anti-integrin-αv heterodimer (e.g., integrin-αvβ1) antibody, a vector comprising the polynucleotide, a host cell comprising the polynucleotide, an immunoconjugate comprising an anti-integrin-αv heterodimer (e.g., integrin-αvβ1) antibody, or any combination thereof.

Certain aspects of the present disclosure are directed to a method of treating a cancer in a subject in need thereof, comprising administering to the subject an effective dose of a composition disclosed herein (e.g., an antibody, polynucleotide, vector, host cell, immunoconjugate, or pharmaceutical composition). In other aspects, the present disclosure is directed to a method of inhibiting shedding of integrin-αv heterodimer (e.g., integrin-αvβ1) by a tumor cell in a subject in need thereof, comprising administering to the subject an effective dose of a composition disclosed herein. In other aspects, the present disclosure is directed to a method of reducing shed integrin-αv heterodimer (e.g., integrin-αvβ1) in the serum and/or retaining integrin-αv heterodimer (e.g., integrin-αvβ1) on the cell surface in a subject in need thereof, comprising administering to the subject an effective dose of a composition disclosed herein. In other aspects, the present disclosure is directed to a method of killing a tumor cell in a subject in need thereof, comprising administering to the subject an effective dose of a composition disclosed herein. In other aspects, the present disclosure is directed to a method of reducing the size of a tumor in a subject in need thereof, comprising administering to the subject an effective dose of a composition disclosed herein. In other aspects, the present disclosure is directed to inhibiting metastasis of a tumor in a subject in need thereof, comprising administering to the subject an effective dose of a composition disclosed herein. In some aspects, the subject is a human.

The compositions of the present disclosure can be administered using any pharmaceutically acceptable route. In some aspects, the composition (e.g., antibody, polynucleotide, vector, host cell, immunoconjugate, or pharmaceutical composition) is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, topically, epidermally, mucosally, or any combination thereof. In some aspects, the composition is administered intravenously. In some aspects, the composition is administered subcutaneously.

In certain aspects, the method reduces the size of a cancer, e.g., the size of a tumor, in the subject. In some aspects, the size of the caner is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%.

In some aspects, the method increases the over survival of the subject. In some aspects, the overall survival is increased relative to the average overall survival of a subject having the same cancer but treated with a different therapy. In certain aspects, the overall survival is increased by at least about 10%, at least about 20%, at least about 25%, at least about 50%, at least about 2 fold, at least about 3 fold, at least about 5 fold. In some aspects, the overall survival is increased by at least about one month, at least about 2 months, at least about 3 months, at least about 4 months, at least about 5 months, at least about 6 months, at least about 7 months, at least about 8 months, at least about 9 months, at least about 10 months, at least about 1 months, at least about 12 months, at least about 15 months, at least about 18 months, at least about 21 months, at least about 2 years, at least about 3 years, at least about 4 years, at least about 5 years, or at least about 10 years.

In some aspects, the method increases the progression free survival of the subject. In some aspects, the overall survival is increased relative to the average progression free survival of a subject having the same cancer but treated with a different therapy. In certain aspects, the progression free survival is increased by at least about 10%, at least about 20%, at least about 25%, at least about 50%, at least about 2 fold, at least about 3 fold, at least about 5 fold. In some aspects, the overall survival is increased by at least about one month, at least about 2 months, at least about 3 months, at least about 4 months, at least about 5 months, at least about 6 months, at least about 7 months, at least about 8 months, at least about 9 months, at least about 10 months, at least about 1 months, at least about 12 months, at least about 15 months, at least about 18 months, at least about 21 months, at least about 2 years, at least about 3 years, at least about 4 years, at least about 5 years, or at least about 10 years.

In some aspects, the method increases the objective response rate of the subject. In certain aspects, the method induces a complete response in the subject. In some aspects, the method induces a partial response in the subject.

In some aspects, the method comprises administering an anti-integrin-αv heterodimer (e.g., integrin-αvβ1) antibody (or a polynucleotide, vector, host cell, or immunoconjugate) disclosed herein and a second therapy. In some aspects, the second therapy is administered prior to the anti-integrin-αv heterodimer (e.g., integrin-αvβ1) antibody. In some aspects, the second therapy is administered after the anti-integrin-αv heterodimer (e.g., integrin-αvβ1) antibody. In some aspects, the second therapy is administered concurrently with the anti-integrin-αv heterodimer (e.g., integrin-αvβ1) antibody. In certain aspects, the anti-integrin-αv heterodimer (e.g., integrin-αvβ1) antibody and the second therapy are administered separately. In other aspects, the anti-integrin-αv heterodimer (e.g., integrin-αvβ1) antibody and the second therapy are administered in a single formulation.

The second therapy can be any other therapy known in the art. In some aspects, the second therapy comprises an immunotherapy. In some aspects, the second therapy comprises a chemotherapy. In some aspects, the second therapy comprises a radiotherapy. In some aspects, the second therapy comprises a surgery. In some aspects, the second therapy comprises administering a second therapeutic agent.

In certain aspects, the second therapeutic agent comprises a second antibody. In some aspects, the second therapeutic agent comprises an effective amount of an antibody that specifically binds a protein selected from Inducible T cell Co-Stimulator (ICOS), CD137 (4-11B), CD134 (OX40), NKG2A, CD27, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), NKG2a, KIR, TGFβ, IL-10, IL-8, B7-H4, Fas ligand, CXCR4, mesothelin, CEACAM-1, CD96, CD52, HER2, and any combination thereof.

XI.A. Anti-PD-1 Antibodies

In some aspects, the second antibody is an anti-PD-1 antibody. Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

XI.B. Anti-PD-L1 Antibodies

In some aspects, the second antibody is an anti-PD-L1 antibody. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

In other aspects, the anti-PD-L1 monoclonal antibody is selected from the group consisting of 28-8, 28-1, 28-12, 29-8, 5H1, and any combination thereof.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

XI.C. Anti-CTLA-4 Antibodies

In some aspects, the second antibody is an anti-CTLA-4 antibody. Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab.

In particular aspects, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

In some aspects, the CTLA-4 antibody is nonfucosylated or hypofucosylated. In some aspects, the CTLA-4 antibody exhibits enhanced ADCC and/or ADCP activity. In some aspects, the CTLA-4 antibody is BMS-986218, as described in PCT/US18/19868.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding portion thereof cross-competes with ipilimumab and/or tremelimumab for binding to human CTLA-4.

XI.D. Anti-LAG-3 Antibodies

In some aspects, the second antibody is an anti-LAG-3 antibody. Anti-LAG-3 antibodies of the instant disclosure bind to human LAG-3. Antibodies that bind to LAG-3 have been disclosed in Int'l Publ. No. WO/2015/042246 and U.S. Publ. Nos. 2014/0093511 and 2011/0150892.

An exemplary LAG-3 antibody useful in the present disclosure is 25F7 (described in U.S. Publ. No. 2011/0150892). An additional exemplary LAG-3 antibody useful in the present disclosure is BMS-986016. In one aspect, an anti-LAG-3 antibody useful for the composition cross-competes with 25F7 or BMS-986016. In another aspect, an anti-LAG-3 antibody useful for the composition binds to the same epitope as 25F7 or BMS-986016. In other aspects, an anti-LAG-3 antibody comprises six CDRs of 25F7 or BMS-986016.

XI.E. Anti-CD137 Antibodies

In some aspects, the second antibody is an anti-CD137 antibody. Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669.

In some aspects, the anti-CD137 antibody is urelumab (BMS-663513), described in U.S. Pat. No. 7,288,638 (20H4.9-IgG4 [10C7 or BMS-663513]). In some aspects, the anti-CD137 antibody is BMS-663031 (20H4.9-IgG1), described in U.S. Pat. No. 7,288,638. In some aspects, the anti-CD137 antibody is 4E9 or BMS-554271, described in U.S. Pat. No. 6,887,673. In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer's PF-05082566 (PF-2566). In other aspects, an anti-CD137 antibody useful for the disclosure cross-competes with the anti-CD137 antibodies disclosed herein. In some aspects, an anti-CD137 antibody binds to the same epitope as the anti-CD137 antibody disclosed herein. In other aspects, an anti-CD137 antibody useful in the disclosure comprises six CDRs of the anti-CD137 antibodies disclosed herein.

XI.F. Anti-KIR Antibodies

In some aspects, the second antibody is an anti-KIR3 antibody. Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448.

One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In one aspect, an anti-KIR antibody for the present composition cross competes for binding to KIR with lirilumab or I-7F9. In another aspect, an anti-KIR antibody binds to the same epitope as lirilumab or I-7F9. In other aspects, an anti-KIR antibody comprises six CDRs of lirilumab or I-7F9.

XI.G. Anti-GITR Antibodies

In some aspects, the second antibody is an anti-GITR antibody. Anti-GITR antibodies may be any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841.

In one aspect, an anti-GITR antibody useful in the present disclosure is TRX518 (described in, for example, Schaer et al. Curr Opin Immunol. (2012) April; 24(2): 217-224, and WO/2006/105021). In another aspect, the anti-GITR antibody is selected from MK4166, MK1248, and antibodies described in WO 11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO 11/028683 or U.S. Pat. No. 8,709,424). In certain aspects, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain aspects, an anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979). In certain aspects, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain aspects, an anti-GITR antibody is an anti-GITR antibody that is described in PCT/US2015/033991 (e.g., an antibody comprising the variable regions of 28F3, 18E10 or 19D3). For example, an anti-GITR antibody is an antibody comprising the following VH and VL chains or the CDRs thereof:

VH:
(SEQ ID NO: 78)
QVQLVESGGGVVQPGRSLRLSCAASGFTFSSYGMHWVRQAPGKGLEWVA
VIWYEGSNKYYADSVKGRFTISRDNSKNTLYLQMNSLRAEDTAVYYCAR
GGSMVRGDYYYGMDVWGQGTTVTVS,
and
VL:
(SEQ ID NO: 79)
AIQLTQSPSSLSASVGDRVTITCRASQGISSALAWYQQKPGKAPKLLIY
DASSLESGVPSRFSGSGSGTDFTLTISSLQPEDFATYYCQQFNSYPYTF
GQGTKLEIK;
or
VH:
(SEQ ID NO: 80)
QVQLVESGGGVVQPGRSLRLSCAASGFTFSSYGFHWVRQAPGKGLEWVA
VIWYAGSNKFYADSVKGRFTISRDNSKNTLYLQMNSLRAEDTAVYYCAR
GGQLDYYYYYVMDVWGQGTTVTVSS,
and
VL:
(SEQ ID NO: 81)
DIQMTQSPSSLSASVGDRVTITCRASQGISSWLAWYQQKPEKAPKSLIY
AASSLQSGVPSRFSGSGSGTDFTLTISSLQPEDFATYYCQQYNSYPYTF
GQGTKLEIK;
or
VH:
(SEQ ID NO: 82)
VQLVESGGGVVQPGRSLRLSCAASGFTFSSYGMHWVRQAPGKGLEWVAV
IWYAGSNKYYADSVKGRFTISRDNSKNTLYLQMNSLRAEDTAVYYCARG
GRIAVAFYYSMDVWGQGTTVTVSS,
and
VL:
(SEQ ID NO: 83)
DIQMTQSPSSLSASVGDRVTITCRASQGISSWLAWYQQKPEKAPKSLIY
AASSLQSGVPSRFSGSGSGTDFTLTISSLQPEDFATYYCQQYNSYPYTF
GQGTKLEIK.

In certain aspects, an antibody comprising a pair of the above VH and VL light chains, or their CDRs, comprises a heavy chain constant region of an IgG1 isotype, either wild type or mutated, e.g., to be effectorless. In one aspect, an anti-GITR antibody comprises the following heavy and light chains amino acid sequences:

heavy chain:
(SEQ ID NO: 84)
QVQLVESGGGVVQPGRSLRLSCAASGFTFSSYGMHWVRQAPGKGLEWVA
VIWYEGSNKYYADSVKGRFTISRDNSKNTLYLQMNSLRAEDTAVYYCAR
GGSMVRGDYYYGMDVWGQGTTVTVSSASTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPEN
NYKTTPPMLDSDGSFFLYSKLTVDKSRWQQGNVFSCSVMHEALHNHYTQ
KSLSLSPG,
and
light chain:
(SEQ ID NO: 85)
AIQLTQSPSSLSASVGDRVTITCRASQGISSALAWYQQKPGKAPKLLIY
DASSLESGVPSRFSGSGSGTDFTLTISSLQPEDFATYYCQQFNSYPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC,
or
heavy chain:
(SEQ ID NO: 86)
qvqlvesgggvvqpgrslrlscaasgftfssygmhwvrqapgkglewva
viwyegsnkyyadsvkgrftisrdnskntlylqmnslraedtavyycar
ggsmvrgdyyygmdvwgqgttvtvssastkgpsvfplapsskstsggta
algclvkdyfpepvtvswnsgaltsgvhtfpavlqssglyslssvvtvp
ssslgtqtyicnvnhkpsntkvdkrvepkscdkthtcppcpapeaegap
svflfppkpkdtlmisrtpevtcvvvdvshedpevkfnwyvdgvevhna
ktkpreeqynstyrvvsvltvlhqdwlngkeykckvsnkalpssiekti
skakgqprepqvytlppsreemtknqvsltclvkgfypsdiavewesng
qpennykttppvldsdgsfflyskltvdksrwqqgnvfscsvmhealhn
hytqkslslspg,
and
light chain:
(SEQ ID NO: 87)
AIQLTQSPSSLSASVGDRVTITCRASQGISSALAWYQQKPGKAPKLLIY
DASSLESGVPSRFSGSGSGTDFTLTISSLQPEDFATYYCQQFNSYPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In certain aspects, the anti-GITR antibody cross-competes with an anti-GITR antibody described herein, e.g., TRX518, MK4166 or an antibody comprising a VH domain and a VL domain amino acid sequence described herein. In some aspects, the anti-GITR antibody binds the same epitope as that of an anti-GITR antibody described herein, e.g., TRX518, MK4166 or an antibody comprising a VH domain and a VL domain amino acid sequence described herein. In certain aspects, the anti-GITR antibody comprises the six CDRs of TRX518, MK4166 or those of an antibody comprising a VH domain and a VL domain amino acid sequence described herein.

XI.H. Anti-TIM3 Antibodies

In some aspects, the second antibody is an anti-TIM3 antibody. In some aspects, the anti-TIM3 antibody is selected from the anti-TIM3 antibodies disclosed in Int'l Publ. Nos. WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802 WO2016068803, WO2016/111947, WO/2017/031242.

XI.I Anti-OX40 Antibodies

In some aspects, the second antibody is an anti-OX40 (also known as CD134, TNFRSF4, ACT35 and/or TXGP1L) antibody. In some aspects, the anti-OX40 antibody is BMS-986178 (Bristol-Myers Squibb Company), described in Int'l Publ. No. WO20160196228. In some aspects, the anti-OX40 antibody is selected from the anti-OX40 antibodies described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182.

XI.J. Anti-NKG2A Antibodies

In some aspects, the second antibody is an anti-NKG2A antibody. NKG2A is a member of the C-type lectin receptor family that is expressed on natural killer (NK) cells and a subset of T lymphocytes. Specifically, NKG2A primarily expressed on tumor infiltrating innate immune effector NK cells, as well as on some CD8+ T cells. Its natural ligand human leukocyte antigen E (HLA-E) is expressed on solid and hematologic tumors. NKG2A is an inhibitory receptor that blinds HLA-E.

In some aspects, the anti-NKG2A antibody is BMS-986315, a human monoclonal antibody that blocks the interaction of NKG2A to its ligand HLA-E, thus allowing activation of an anti-tumor immune response. In some aspects, the anti-NKG2A antibody is a checkpoint inhibitor that activates T cells, NK cells, and/or tumor-infiltrating immune cells. In some aspects, the anti-NKG2A antibody is selected from the anti-NKG2A antibodies described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Pat. No. 9,447,185 (Innate Pharma S/A; Novo Nordisk A/S; University of Genova); WO 2008/009545 (Novo Nordisk A/S); U.S. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945 (Academisch Ziekenhuis Leiden H.O.D.N. LUMC); WO 2016/041947 (Innate Pharma); and WO 2016/041945 (Innate Pharma).

XI.K. Anti-ICOS ANTIBODIES

In some aspects, the second antibody is an anti-ICOS antibody. ICOS is an immune checkpoint protein that is a member of the CD28-superfamily. ICOS is a 55-60 kDa type I transmembrane protein that is expressed on T cells after T cell activation and co-stimulates T-cell activation after binding its ligand, ICOS-L (B7H2). ICOS is also known as inducible T-cell co-stimulator, CVID1, AILIM, inducible costimulator, CD278, activation-inducible lymphocyte immunomediatory molecule, and CD278 antigen.

In some aspects, the anti-ICOS antibody is BMS-986226, a humanized IgG monoclonal antibody that binds to and stimulates human ICOS. In some aspects, the anti-ICOS antibody is selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited).

XI.L. Anti-TIGIT Antibodies

In some aspects, the second antibody is an anti-TIGIT antibody. In some aspects, the anti-TIGIT antibody is BMS-986207. In some aspects, the anti-TIGIT antibody is clone 22C2, as described in WO 2016/106302. In some aspects, the anti-TIGIT antibody is MTIG7192A/RG6058/RO7092284, or clone 4.1D3, as described in WO 2017/053748. In some aspects, the anti-TIGIT antibody is selected from the anti-TIGIT antibodies described in, for example, WO 2016/106302 (Bristol-Myers Squibb Company) and WO 2017/053748 (Genentech).

XI.M. Additional Anti-Cancer Agents

In some aspects, the anti-integrin-αv heterodimer (e.g., integrin-αvβ1) antibody is used in combination with an anti-IL-10 antibody. In some aspects, the anti-integrin-αv heterodimer (e.g., integrin-αvβ1) antibody is used in combination with a long-acting IL-10 molecule. In some aspects, the long-acting IL-10 molecule may be an IL-10-Fc fusion molecule. In some aspects, the long-acting IL-10 molecule may be a Pegylated IL-10, such as AM0010 (ARMO BioSciences).

In some aspects, the anti-integrin-αv heterodimer (e.g., integrin-αvβ1) antibody is used in combination with an anti-IL-2 antibody. In some aspects, the anti-integrin-αv heterodimer (e.g., integrin-αvβ1) antibody is used in combination with a long-acting IL-2 molecule. In some aspects, the long-acting IL-2 may be a Pegylated IL-2, such as NKTR-214 (Nektar; see U.S. Pat. No. 8,252,275, WO12/065086 and WO15/125159).

In some aspects, the anti-integrin-αv heterodimer (e.g., integrin-αvβ1) antibody is used in combination with an anti-VISTA antibody.

In some aspects, the anti-integrin-αv heterodimer (e.g., integrin-αvβ1) antibody is used in combination with an anti-CD96 antibody.

In some aspects, the anti-integrin-αv heterodimer (e.g., integrin-αvβ1) antibody is used in combination with an anti-IL-8 antibody, e.g., with HuMax®-IL8.

In some aspects, the anti-integrin-αv heterodimer (e.g., integrin-αvβ1) antibody is used in combination with an anti-TGFβ antibody.

In some other aspects, the anti-integrin-αv heterodimer (e.g., integrin-αvβ1) antibody is used in combination with an anti-B7-H4 antibody. In certain aspects, the anti-B7-H4 antibody is an anti-B7-H4 disclosed in Int'l Publ. No. WO/2009/073533.

In certain aspects, the anti-integrin-αv heterodimer (e.g., integrin-αvβ1) antibody is used in combination with an anti-Fas ligand antibody. In certain aspects, the anti-Fas ligand antibody is an anti-Fas ligand disclosed in Int'l Publ. No. WO/2009/073533.

In some aspects, the anti-integrin-αv heterodimer (e.g., integrin-αvβ1) antibody is used in combination with an anti-CXCR4 antibody. In certain aspects, the anti-CXCR4 antibody is an anti-CXCR4 disclosed in U.S. Publ. No. 2014/0322208 (e.g., Ulocuplumab (BMS-936564)).

In some aspects, the anti-integrin-αv heterodimer (e.g., integrin-αvβ1) antibody is used in combination with an anti-mesothelin antibody. In certain aspects, the anti-mesothelin antibody is an anti-mesothelin disclosed in U.S. Pat. No. 8,399,623.

In some aspects, the anti-integrin-αv heterodimer (e.g., integrin-αvβ1) antibody is used in combination with an anti-HER2 antibody. In certain aspects, the anti-HER2 antibody is Herceptin (U.S. Pat. No. 5,821,337), trastuzumab, or ado-trastuzumab emtansine (Kadcyla, e.g., WO/2001/000244).

In aspects, the anti-integrin-αv heterodimer (e.g., integrin-αvβ1) antibody is used in combination with an anti-CD27 antibody. In aspects, the anti-CD-27 antibody is Varlilumab (also known as “CDX-1127” and “1F5”), which is a human IgG1 antibody that is an agonist for human CD27, as disclosed in, for example, U.S. Pat. No. 9,169,325.

In some aspects, the anti-integrin-αv heterodimer (e.g., integrin-αvβ1) antibody is used in combination with an anti-CD73 antibody. In certain aspects, the anti-CD73 antibody is CD73.4.IgG2C219S.IgG1. If.

In certain aspects, the second therapy comprises administering a chemotherapeutic agent. In some aspects, the chemotherapeutic agent induces integrin-αv heterodimer (e.g., integrin-αvβ1) expression on tumor cells. In some aspects, the chemotherapeutic agent is selected from a proteasome inhibitor, an immunomodulatory drug (IMiD), a Bet inhibitor, and any combination thereof. In some aspects, the proteasome inhibitor is selected from bortezomib, ixazomib, carfilzomib, oprozomib and marizomib. In certain aspects, the proteasome inhibitor comprises bortezomib.

In some aspects, the second therapy comprises a radiotherapy. Any radiotherapy known in the art can be used as the second therapy.

In some aspects, the second therapy comprises administering an agent that activates innate immune cells. In some aspects, the agent that activates innate immune cells comprises an NLRP3 agonist. In some aspects, the NLRP3 agonist comprises monosodium urate monohydrate (MSU) and/or the vaccine adjuvant alum. In some aspects, the agent that activates innate immune cells is a toll like receptor 7 (TLR7) agonist. In some aspects, the TLR7 agonist comprises imiquimod (R837), GS-9620 (see Tsai et al., J. Virology doi:10.1128/JVI.02166-16 (Feb. 8, 2017)), ORN R-2336 (Miltenyl Biotec), or any combination thereof.

In some aspects, the second therapy comprises administering an agent that enhances the survival of natural killer (NK) cells, CD8+ T cells, or both. In some aspects, the agent comprises IL-2. In certain aspects, the agent comprises pegylated IL-2.

In certain aspects, the second therapy comprises administering an agent selected from the group consisting of doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, vincristine (ONCOVIN®), fludarabine (FLUDARA®), thalidomide (THALOMID®), alemtuzumab (CAMPATH®), ofatumumab (ARZERRA®), everolimus (AFINITOR®, ZORTRESS®), carfilzomib (KYPROLIS™), and any combination thereof.

XI.N. Cancer

Anti-integrin-αv heterodimer (e.g., integrin-αvβ1) antibodies can enhance the immune response to cancerous cells in a patient having cancer. Provided herein are methods for treating a subject having cancer, comprising administering to the subject an anti-integrin-αv heterodimer (e.g., integrin-αvβ1) antibody described herein, such that the subject is treated, e.g., such that growth of cancerous tumors is inhibited or reduced and/or that the tumors regress and/or that prolonged survival is achieved. An anti-integrin-αv heterodimer (e.g., integrin-αvβ1) antibody can be used alone to inhibit the growth of cancerous tumors. Alternatively, an anti-integrin-αv heterodimer (e.g., integrin-αvβ1) antibody can be used in conjunction with another agent, e.g., another immunogenic agent, a standard cancer treatment, or another antibody, as described below.

Accordingly, provided herein are methods of treating cancer, e.g., by inhibiting growth of tumor cells, in a subject, comprising administering to the subject a therapeutically effective amount of an anti-integrin-αv heterodimer (e.g., integrin-αvβ1) antibody described herein, e.g., MICA.36, MICA.52, MICA.54, MICA.2, and 71C2 having a wild type IgG constant region or a constant region variant having altered effector function, or antigen-binding portion thereof. The antibody can be a human anti-integrin-αv heterodimer (e.g., integrin-αvβ1) antibody (such as any of the human anti-human integrin-αv heterodimer (e.g., integrin-αvβ1) antibodies described herein). Cancers whose growth can be inhibited using the antibodies of the disclosure include cancers typically responsive to immunotherapy and those that are not typically responsive to immunotherapy. Cancers that can be treated also include integrin-αv heterodimer (e.g., integrin-αvβ1) positive cancers. Cancers can be cancers with solid tumors or hematolotical malignancies (liquid tumors). Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, rectal cancer, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin (e.g., human papilloma virus (HPV-related or -originating tumors)); and any combinations of said cancers.

Non-limiting examples of cancers for treatment include hematological malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; and any combinations of said cancers.

The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In some aspects, the subject has a cancer selected from non-small cell lung cancer (NSCLC), head and neck squamous cell carcinoma (HNSCC), melanoma, bladder cancer, pancreatic cancer, gastric cancer, colon cancer, renal cell carcinoma (RCC), small-cell lung cancer (SCLC), mesothelioma, hepatocellular carcinoma, prostate cancer, multiple myeloma, and combinations of said cancers.

In some aspects, the subject has a cancer selected from non-small cell lung cancer (NSCLC), head and neck squamous cell carcinoma (HNSCC), melanoma, bladder cancer, pancreatic cancer, gastric cancer, colon cancer, and combinations of said cancers.

In some aspects, an anti-integrin-αv heterodimer (e.g., integrin-αvβ1) antibody is administered to patients having a cancer that exhibited an inadequate response to, or progressed on, a prior treatment, e.g., a prior treatment with an immuno-oncology or immunotherapy drug, or patients having a cancer that is refractory or resistant, either intrinsically refractory or resistant (e.g., refractory to a PD-1 pathway antagonist), or a wherein the resistance or refractory state is acquired. For example, subjects who are not responsive or not sufficiently responsive to a first therapy or who see disease progression following treatment, e.g., anti-PD-1 treatment, can be treated by administration of an anti-integrin-αv heterodimer (e.g., integrin-αvβ1) antibody alone or in combination with another therapy (e.g., with an anti-PD-1 therapy).

In some aspects, an anti-integrin-αv heterodimer (e.g., integrin-αvβ1) antibody is administered to patients who have not previously received (i.e., been treated with) an immuno-oncology agent, e.g., a PD-1 pathway antagonist.

In some aspects, a method of treating cancer in a subject comprises first determining whether the subject is integrin-αv heterodimer (e.g., integrin-αvβ1) positive, e.g., has tumor cells that express integrin-αv heterodimer (e.g., integrin-αvβ1), and if the subject has integrin-αv heterodimer (e.g., integrin-αvβ1) positive cancer, then administering to the subject an anti-integrin-αv heterodimer (e.g., integrin-αvβ1) antibody, e.g., described herein. A method of treating a subject having cancer with an anti-integrin-αv heterodimer (e.g., integrin-αvβ1) antibody may comprise administering to a subject who has cancer cells that express integrin-αv heterodimer (e.g., integrin-αvβ1), a therapeutically effective amount of a integrin-αv heterodimer (e.g., integrin-αvβ1) antibody. Also provided herein are methods for predicting whether a subject will respond to treatment with an anti-integrin-αv heterodimer (e.g., integrin-αvβ31) antibody, wherein the methods comprise determining the level of integrin-αv heterodimer (e.g., integrin-αvβ1) in cancer cells of the patient, and if cancer cells of the subject are integrin-αv heterodimer (e.g., integrin-αvβ1) positive, then the subject is likely to respond to a treatment with a integrin-αv heterodimer (e.g., integrin-αvβ1) antibody.

In some aspects, a method of treating cancer in a subject comprises first determining whether the subject is PD-L1 or PD-1 positive, e.g., has tumor cells or TILs that express PD-L1 or PD-1, and if the subject has PD-L1 or PD-1 positive cancer or TIL cells, then administering to the subject an anti-integrin-αv heterodimer (e.g., integrin-αvβ1) antibody (and optionally a PD-1 or PD-L1 antagonist), e.g., described herein. A method of treating a subject having cancer with an anti-integrin-αv heterodimer (e.g., integrin-αvβ1) antibody (and optionally a PD-1 or PD-L1 antagonist) may comprise administering to a subject who has cancer cells or TIL cells that express PD-L1 or PD-1, a therapeutically effective amount of an anti-integrin-αv heterodimer (e.g., integrin-αvβ1) antibody (and optionally a PD-1 or PD-L1 antagonist). Also provided herein are methods for predicting whether a subject will respond to treatment with an anti-integrin-αv heterodimer (e.g., integrin-αvβ1) antibody (and optionally a PD-1 or PD-L1 antagonist), wherein the methods comprise determining the level of PD-L1 or PD-1 in cancer or TIL cells of the patient, and if cancer or TIL cells of the subject are PD-L1 or PD-1 positive, then the subject is likely to respond to a treatment with a MICA antibody (and optionally a PD-1 or PD-L1 antagonist).

An anti-integrin-αv heterodimer (e.g., integrin-αvβ1) antibody can be administered with a standard of care treatment. An anti-integrin-αv heterodimer (e.g., integrin-αvβ1) antibody can be administered as a maintenance therapy, e.g., a therapy that is intended to prevent the occurrence or recurrence of tumors.

An anti-integrin-αv heterodimer (e.g., integrin-αvβ1) antibody can be administered with another treatment, e.g., radiation, surgery, or chemotherapy. For example, anti-integrin-αv heterodimer (e.g., integrin-αvβ1) antibody adjunctive therapy can be administered when there is a risk that micrometastases can be present and/or in order to reduce the risk of a relapse.

An anti-integrin-αv heterodimer (e.g., integrin-αvβ1) antibody can be administered as a monotherapy, or as the only immuno stimulating therapy. Antibodies to integrin-αv heterodimer (e.g., integrin-αvβ1), e.g., the anti-integrin-αv heterodimer (e.g., integrin-αvβ1), can also be combined with an immunogenic agent, such as cancerous cells, purified tumor antigens (including recombinant proteins, peptides, and carbohydrate molecules), cells, and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below).

In humans, some tumors have been shown to be immunogenic such as melanomas. By lowering the threshold of T cell activation by reducing the level of soluble integrin-αv heterodimer (e.g., integrin-αvβ1) in plasma, the tumor responses in the host can be activated, allowing treatment of non-immunogenic tumors or those having limited immunogenicity.

An anti-integrin-αv heterodimer (e.g., integrin-αvβ1) antibody, e.g., an anti-integrin-αv heterodimer (e.g., integrin-αvβ1) antibody described herein, can be combined with a vaccination protocol. Many experimental strategies for vaccination against tumors have been devised (see Rosenberg, S., 2000, Development of Cancer Vaccines, ASCO Educational Book Spring: 60-62; Logothetis, C, 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. Anti-integrin-αv heterodimer (e.g., integrin-αvβ1) antibody treatment can be used in conjunction with a collection of recombinant proteins and/or peptides expressed in a tumor in order to generate an immune response to these proteins. These proteins are normally viewed by the immune system as self-antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen, which can be used in conjunction with administration of an anti-integrin-αv heterodimer (e.g., integrin-αvβ1) antibody, is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269: 1585-1588; Tamura et al. (1997) Science 278: 117-120).

Dendritic cells (DC) are potent antigen presenting cells that can be used to prime antigen-specific responses. DCs can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with administration of an anti-integrin-αv heterodimer (e.g., integrin-αvβ1) antibody to activate more potent anti-tumor responses.

Administration of an anti-integrin-αv heterodimer (e.g., integrin-αvβ1) antibody can also be combined with standard cancer treatments (e.g., surgery, radiation, and chemotherapy). Administration of an anti-integrin-αv heterodimer (e.g., integrin-αvβ1) antibody can be effectively combined with chemotherapeutic regimes. In these instances, it can be possible to reduce the dose of chemotherapeutic reagent administered (Mokyr et al. (1998) Cancer Research 58: 5301-5304). An example of such a combination is an anti-integrin-αv heterodimer (e.g., integrin-αvβ1) antibody in combination with decarbazine for the treatment of melanoma. Another example of such a combination is an anti-integrin-αv heterodimer (e.g., integrin-αvβ1) antibody in combination with interleukin-2 (IL-2), e.g. pegyalated IL-2, for the treatment of melanoma. The scientific rationale behind the combined use of an anti-integrin-αv heterodimer (e.g., integrin-αvβ1) antibody and chemotherapy is that cell death, that is a consequence of the cytotoxic action of most chemotherapeutic compounds, should result in increased levels of tumor antigen in the antigen presentation pathway. Other combination therapies that can result in synergy with administration of an anti-integrin-αv heterodimer (e.g., integrin-αvβ1) antibody through cell death are radiation, surgery, and hormone deprivation. Each of these protocols creates a source of tumor antigen in the host. Angiogenesis inhibitors can also be combined with administration of an anti-integrin-αv heterodimer (e.g., integrin-αvβ1) antibody. Inhibition of angiogenesis leads to tumor cell death which can feed tumor antigen into host antigen presentation pathways.

The anti-integrin-αv heterodimer (e.g., integrin-αvβ1) antibodies described herein can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti-tumor antigen (e.g., Her-2/neu) bispecific antibodies have been used to target macrophages to sites of tumor. This targeting can more effectively activate tumor specific responses. The T cell arm of these responses would be augmented by the action of the anti-integrin-αv heterodimer (e.g., integrin-αvβ1) antibody. Alternatively, antigen can be delivered directly to DCs by the use of bispecific antibodies which bind to tumor antigen and a dendritic cell specific cell surface marker.

Tumors evade host immune surveillance by a large variety of mechanisms. Many of these mechanisms can be overcome by the inactivation of proteins which are expressed by the tumors and which are immunosuppressive. These include among others TGF-β (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with anti-integrin-αv heterodimer (e.g., integrin-αvβ1) antibodies to counteract the effects of the immunosuppressive agent and favor tumor immune responses by the host.

Other antibodies which activate host immune responsiveness can be used in combination with anti-integrin-αv heterodimer (e.g., integrin-αvβ1) antibodies. These include molecules on the surface of dendritic cells which activate DC function and antigen presentation. Anti-CD40 antibodies are able to substitute effectively for T cell helper activity (Ridge et al. (1998) Nature 393: 474-478) and can be used in conjunction with anti-integrin-αv heterodimer (e.g., integrin-αvβ1) antibodies. Activating antibodies to T cell costimulatory molecules such as CTLA-4 (e.g., U.S. Pat. No. 5,811,097), OX-40 (Weinberg et al. (2000) Immunol 164: 2160-2169), 4-1BB (Melero et al. (1997) Nature Medicine 3: 682-685 (1997), and ICOS (Hutloff et al. (1999) Nature 397: 262-266) can also provide for increased levels of T cell activation. Inhibitors of PD1 or PD-L1 can also be used in conjunction with an anti-integrin-αv heterodimer (e.g., integrin-αvβ1) antibody. Other combination are provided elsewhere herein.

Bone marrow transplantation is currently being used to treat a variety of tumors of hematopoietic origin. While graft versus host disease is a consequence of this treatment, therapeutic benefit can be obtained from graft vs. tumor responses. integrin-αv heterodimer (e.g., integrin-αvβ1) inhibition can be used to increase the effectiveness of the donor engrafted tumor specific T cells.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285: 546-51). These methods can also be used to activate T cell responses to infectious agents such as CMV. Ex vivo activation in the presence of anti-integrin-αv heterodimer (e.g., integrin-αvβ1) antibodies can increase the frequency and activity of the adoptively transferred T cells.

III.B. Combination Therapies

In addition to the combinations therapies provided above, anti-integrin-αv heterodimer (e.g., integrin-αvβ1) antibodies, e.g., those described herein, can also be used in combination therapy, e.g., for treating cancer, as described below.

Provided herein are methods of combination therapy in which an anti-integrin-αv heterodimer (e.g., integrin-αvβ1) antibody is coadministered with one or more additional agents, e.g., small molecule drugs, antibodies or antigen binding portions thereof, that are effective in stimulating immune responses to thereby further enhance, stimulate or upregulate immune responses in a subject.

Generally, an anti-integrin-αv heterodimer (e.g., integrin-αvβ1) antibody, e.g., described herein, can be combined with (i) an agonist of a stimulatory (e.g., co-stimulatory) molecule (e.g., receptor or ligand) and/or (ii) an antagonist of an inhibitory signal or molecule (e.g., receptor or ligand) on immune cells, such as T cells, both of which result in amplifying immune responses, such as antigen-specific T cell responses. In some aspects, an immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on cells, e.g., those inhibiting T cell activation or those involved in innate immunity, e.g., NK cells, and wherein the immuno-oncology agent enhances innate immunity. Such immuno-oncology agents are often referred to as immune checkpoint regulators, e.g., immune checkpoint inhibitor or immune checkpoint stimulator.

In some aspects, an anti-integrin-αv heterodimer (e.g., integrin-αvβ1) antibody is administered with an agent that targets a stimulatory or inhibitory molecule that is a member of the immunoglobulin super family (IgSF). For example, anti-integrin-αv heterodimer (e.g., integrin-αvβ1) antibodies, e.g., described herein, can be administered to a subject with an agent that targets a member of the IgSF family to increase an immune response. For example, an anti-integrin-αv heterodimer (e.g., integrin-αvβ31) antibody can be administered with an agent that targets (or binds specifically to) a member of the B7 family of membrane-bound ligands that includes B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor or ligand binding specifically to a B7 family member.

An anti-integrin-αv heterodimer (e.g., integrin-αvβ31) antibody can also be administered with an agent that targets a member of the TNF and TNFR family of molecules (ligands or receptors), such as CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fn 14, TWEAK, BAFFR, EDAR, XEDAR, TACI, APRIL, BCMA, LTpR, LIGHT, DcR3, HVEM, VEGI/TL1A, TRAMP/DR3, EDA1, EDA2, TNFR1, Lymphotoxin a/TNFp, TNFR2, TNFa, LTpR, Lymphotoxin a 102, FAS, FASL, RELT, DR6, TROY, and NGFR (see, e.g., Tansey (2009) Drug Discovery Today 00: 1).

In some aspects, the method comprises administering an anti-integrin-αv heterodimer (e.g., integrin-αvβ1) antibody disclosed herein and one or more of the following agents:

(1) An antagonist (inhibitor or blocking agent) of a protein that inhibits T cell activation (e.g., immune checkpoint inhibitors), such as CTLA-4, PD-1, PD-L1, PD-L2, GITR, and LAG-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, TIM-1, TIM-3, and TIM-4; and/or

(2) An agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H.

Exemplary agents that modulate one of the above proteins and can be combined with anti-integrin-αv heterodimer (e.g., integrin-αvβ1) antibodies, e.g., those described herein, for treating cancer, include: YERVOY® (ipilimumab) or Tremelimumab (to CTLA-4), galiximab (to B7.1), BMS-936558 (to PD-1), MK-3475 (to PD-1), atezolizumab (TECENTRIQ®), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, or any combination thereof.

Other molecules that can be combined with anti-integrin-αv heterodimer (e.g., integrin-αvβ1) antibodies for the treatment of cancer include antagonists of inhibitory receptors on NK cells or agonists of activating receptors on NK cells. For example, anti-integrin-αv heterodimer (e.g., integrin-αvβ1) antibodies can be combined with antagonists of KIR (e.g., lirilumab).

T cell activation is also regulated by soluble cytokines, and anti-integrin-αv heterodimer (e.g., integrin-αvβ1) antibodies can be administered to a subject, e.g., having cancer, with antagonists of cytokines that inhibit T cell activation or agonists of cytokines that stimulate T cell activation.

In some aspects, anti-integrin-αv heterodimer (e.g., integrin-αvβ1) antibodies can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

Anti-integrin-αv heterodimer (e.g., integrin-αvβ1) antibodies can also be administered with agents that inhibit TGF-β signaling.

Additional agents that can be combined with an anti-integrin-αv heterodimer (e.g., integrin-αvβ1) antibody include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that can be combined with an anti-integrin-αv heterodimer (e.g., integrin-αvβ1) antibody include therapies that deplete or block Treg cells, e.g., an agent that specifically binds to CD25.

Another therapy that can be combined with an anti-integrin-αv heterodimer (e.g., integrin-αvβ1) antibody is a therapy that inhibits a metabolic enzyme such as indoleamine dioxigenase (IDO), dioxigenase, arginase, or nitric oxide synthetase. Suitable IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) or F001287.

Another class of agents that can be used with an anti-integrin-αv heterodimer (e.g., integrin-αvβ1) antibody includes agents that inhibit the formation of adenosine, e.g., CD73 inhibitors, or inhibit the adenosine A2A receptor.

Other therapies that can be combined with an anti-integrin-αv heterodimer (e.g., integrin-αvβ1) antibody for treating cancer include therapies that reverse/prevent T cell anergy or exhaustion and therapies that trigger an innate immune activation and/or inflammation at a tumor site.

An anti-integrin-αv heterodimer (e.g., integrin-αvβ1) antibody can be combined with more than one immuno-oncology agent, and can be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells; a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

Anti-integrin-αv heterodimer (e.g., integrin-αvβ1) antibodies described herein can be used together with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some aspects, an anti-integrin-αv heterodimer (e.g., integrin-αvβ1) antibody is administered to a subject together with a BRAF inhibitor if the subject is BRAF V600 mutation positive.

In some aspects, the anti-integrin-αv heterodimer (e.g., integrin-αvβ1) antibody is administered to a subject together with an antibody that specifically binds PD-1, PD-L1, CTLA-4, LAG3, TIGIT, TIM3, NKG2a, OX40, ICOS, CD137, KIR, TGFβ, IL-10, IL-8, IL-2, B7-H4, Fas ligand, CXCR4, mesothelin, CD27, VISTA, CD96, GITR or any combination thereof.

The anti-integrin-αv heterodimer (e.g., integrin-αvβ1) antibodies and combination therapies described herein can also be used in conjunction with other well-known therapies that are selected for their particular usefulness against the indication being treated (e.g., cancer). Combinations of the anti-integrin-αv heterodimer (e.g., integrin-αvβ1) antibodies described herein can be used sequentially with known pharmaceutically acceptable agent(s).

For example, the anti-integrin-αv heterodimer (e.g., integrin-αvβ1) antibodies and combination therapies described herein can be used in combination (e.g., simultaneously or separately) with an additional treatment, such as irradiation and/or chemotherapy, e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, or camptothecin+apo21/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al, Cancer Research 2005; 65:4799-808), c-FLIP (cellular FLICE-inhibitory protein) modulators (e.g., natural and synthetic ligands of PPARy (peroxisome proliferator-activated receptor 7), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3P inhibitors, IAP inhibitors and/or genotoxic drugs.

The anti-integrin-αv heterodimer (e.g., integrin-αvβ1) antibodies and combination therapies described herein can further be used in combination with one or more anti-proliferative cytotoxic agents. Classes of compounds that can be used as anti-proliferative cytotoxic agents include, but are not limited to, the following: Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with anti-integrin-αv heterodimer (e.g., integrin-αvβ1) antibodies, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone BIO, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with anti-integrin-αv heterodimer (e.g., integrin-αvβ1) antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX®, can also be administered to the patient. When employing the methods or compositions described herein, other agents used in the modulation of tumor growth or metastasis in a clinical setting, such as antimimetics, can also be administered as desired.

In some aspects, the combination of the anti-integrin-αv heterodimer (e.g., integrin-αvβ1) antibody and a second agent discussed herein can be administered concurrently as a single composition in a pharmaceutically acceptable carrier, or concurrently as separate compositions with the anti-integrin-αv heterodimer (e.g., integrin-αvβ1) antibody and the second agent in a pharmaceutically acceptable carrier. In some aspects, the combination of the anti-integrin-αv heterodimer (e.g., integrin-αvβ1) antibody and the second agent can be administered sequentially. The administration of the two agents can start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent can start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In some aspects, an anti-neoplastic antibody that can be combined with an anti-integrin-αv heterodimer (e.g., integrin-αvβ1) antibody and/or a second agent includes RITUXAN® (rituximab), HERCEPTIN® (trastuzumab), BEXXAR® (tositumomab), ZEVALIN® (ibritumomab), CAMPATH® (alemtuzumab), LYMPHOCIDE® (eprtuzumab), AVASTIN® (bevacizumab), and TARCEVA® (erlotinib), or any combination thereof. In some aspects, the second antibody useful for the combination therapy with an anti-integrin-αv heterodimer (e.g., integrin-αvβ1) antibody can be an antibody drug conjugate.

In some aspects, an anti-integrin-αv heterodimer (e.g., integrin-αvβ1) antibody alone or in combination with another agent is used concurrently or sequentially with bone marrow transplantation to treat a variety of tumors of hematopoietic origin.

Provided herein are methods for altering an adverse event associated with treatment of a hyperproliferative disease (e.g., cancer) with an immuno stimulatory agent, comprising administering an anti-integrin-αv heterodimer (e.g., integrin-αvβ1) antibody with or without a second agent, to a subject. For example, the methods described herein provide for a method of reducing the incidence of immuno stimulatory therapeutic antibody-induced colitis or diarrhea by administering a non-absorbable steroid to the patient. As used herein, a “non-absorbable steroid” is a glucocorticoid that exhibits extensive first pass metabolism such that, following metabolism in the liver, the bioavailability of the steroid is low, i.e., less than about 20%. In some aspects described herein, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. In some aspects, an anti-integrin-αv heterodimer (e.g., integrin-αvβ1) antibody in conjunction with a non-absorbable steroid can be further combined with a salicylate. Salicylates include 5-ASA agents such as, for example: sulfasalazine (AZULFIDINE®, Pharmacia & Up John); olsalazine (DJPENTUM®, Pharmacia & Up John); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay).

III.C. Methods of Making Anti-Integrin αv Heterodimer Antibodies

Monoclonal anti-integrin αv heterodimer (e.g., integrin-αvβ1) antibodies described herein can be produced using a variety of known techniques, such as the standard somatic cell hybridization technique described by Kohler and Milstein, Nature 256: 495 (1975). Although somatic cell hybridization procedures are preferred, in principle, other techniques for producing monoclonal antibodies also can be employed, e.g., viral or oncogenic transformation of B lymphocytes, phage display technique using libraries of human antibody genes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized anti-integrin αv heterodimer (e.g., integrin-αvβ1) antibodies described herein can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see, e.g., U.S. Pat. No. 4,816,567 to Cabilly et al.). To create a humanized antibody, the murine CDR regions can be inserted into a human framework using methods known in the art (see, e.g., U.S. Pat. No. 5,225,539 to Winter, and U.S. Pat. Nos. 5,530,101; 5,585,089; 5,693,762 and 6,180,370 to Queen et al).

In some aspects, the anti-integrin αv heterodimer (e.g., integrin-αvβ1) antibodies described herein are human monoclonal antibodies. Such human monoclonal antibodies directed against integrin αv heterodimer (e.g., integrin-αvβ1) can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HUMAB-MOUSE® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see, e.g., Lonberg, et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGK monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al., (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) Immunol. 152:2912-2920; Taylor, L. et al. (1994) International Immunology 6: 579-591; and Fishwild, D. et al. (1996) Nature Biotechnology 14: 845-851. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In some aspects, the anti-integrin αv heterodimer (e.g., integrin-αvβ1) antibodies described herein are raised using a mouse that carries human immunoglobulin sequences on transgenes and transchomosomes, such as a mouse that carries a human heavy chain transgene and a human light chain transchromosome. Such mice, referred to herein as “KM mice,” are described in detail in PCT Publication WO 02/43478 to Ishida et al.

Still further, alternative transgenic animal systems expressing human immunoglobulin genes are available in the art and can be used to raise anti-integrin αv heterodimer (e.g., integrin-αvβ1) antibodies described herein. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-integrin αv heterodimer (e.g., integrin-αvβ1) antibodies described herein. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-integrin αv heterodimer (e.g., integrin-αvβ1) antibodies described herein.

Additional mouse systems described in the art for raising human antibodies, e.g., human anti-integrin αv heterodimer (e.g., integrin-αvβ1) antibodies, include (i) the VELOCLMMUNE® mouse (Regeneron Pharmaceuticals, Inc.), in which the endogenous mouse heavy and light chain variable regions have been replaced, via homologous recombination, with human heavy and light chain variable regions, operatively linked to the endogenous mouse constant regions, such that chimeric antibodies (human V/mouse C) are raised in the mice, and then subsequently converted to fully human antibodies using standard recombinant DNA techniques; and (ii) the MEMO® mouse (Merus Biopharmaceuticals, Inc.), in which the mouse contains unrearranged human heavy chain variable regions but a single rearranged human common light chain variable region. Such mice, and use thereof to raise antibodies, are described in, for example, WO 2009/15777, US 2010/0069614, WO 2011/072204, WO 2011/097603, WO 2011/163311, WO 2011/163314, WO 2012/148873, US 2012/0070861 and US 2012/0073004.

Human monoclonal anti-integrin αv heterodimer (e.g., integrin-αvβ1) antibodies described herein can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal anti-integrin αv heterodimer (e.g., integrin-αvβ1) antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1: Antibody Generation and Screening

Cell Lines and Culture Practices

The Chinese hamster ovary (CHO) integrin expressing cells were generated using a transposon based system that stably introduced ORF cDNA target gene sequences. The cells were cultured in Dulbecco's Modified Eagle medium (DMEM) with 10% fetal bovine serum (FBS). The cancer lung panel was obtained from the American Type Culture Collection (ATCC), and the cells were cultured according to ATCC procedures.

Ab Selections on Cellular Antigen

Fab-Phage were cycled through four rounds of binding selection using an antigen negative cell line as the background depleting step, and an antigen overexpressing cell line as the target selection step. The adherent cell lines were brought to suspension using phosphate-buffered saline (PBS) plus 10 mM ethylenediaminetetraacetic acid (EDTA). 10 million re-suspended cells were incubated under gentle rotation for 2 hours at 4° C. in 1 mL of cell growth medium with a library of 3×10¹³ Fab-phage. The Fab-phage were cycled from the background depleting cells (CHO parental) to the target selection cells (CHO AvB1) utilizing a temperature controlled micro-centrifuge to gently pellet the cells and transfer the supernatant. After labelling of the target antigen expressing cells, the cells were washed four times utilizing chilled PBS buffer supplemented with 1% bovine serum albumin (BSA). After the last cell wash, the Fab-phage were eluted from the cellular pellet using 0.1 M hydrochloric acid, incubated for 10 minutes at room temperature, and subsequently neutralized using 11M Tris (tris(hydroxymethyl)aminomethane) buffer. The cellular debris was removed by high-speed centrifugation and clear Fab-phage Elute transferred to a clean vial. Note, in round 4 the Fab-phage were eluted from the pellets of both lines (antigen negative and positive cells) to be analyzed using next-generation sequencing (NGS) methods.

Fab-Phage Amplification

After each selection round, a volume of 500 μl of Fab-phage eluate was used to inoculate 5 ml of OmniMAX E. coli cells in 2YT media at O.D. 600. The cells were incubated with the Fab-phage for 30 minutes at 37° C., 200 RPM. Subsequently, the cells were inoculated with M13KO7 helper phage at a final concentration of 1×108 pfu/ml and incubated at 37° C. for 45 minutes, 200 RPM. The cells were afterward transferred to a shaker flask and brought to a final volume of 50 ml 2YT Media supplemented with 100 pg/ml of carbenicillin and 25 pg/ml kanamycin and grown overnight at 37° C., 200 RPM. The next day the cells were centrifuged and the supernatant was transferred to a clean tube containing ⅕ volume of a solution of 20% polyethylene-glycol-8000 and 2.5 M sodium chloride. The supernatant was incubated at 4° C. for 20 minutes and then centrifuged at 13,000×g for 20 minutes at 4° C. The precipitated Fab-phage pellet was resuspended using PBS supplemented with 1% BSA and stored at 4° C.

Expression and Purification of Fab and IgG Proteins

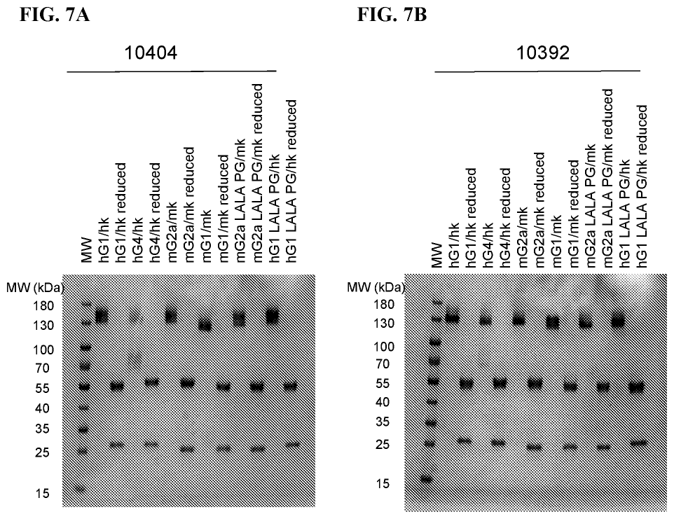

Fab DNA sequences were PCR amplified from Fab-phage phagemid or DNA synthesized. The Fab regions were subcloned into a modified pET21 protein expression plasmid, where the variable light and heavy regions were introduced into an operon cassette resulting in the periplasmic expression of Fabs. Consequently, the plasmids were transformed and expressed in Escherichia coli BL21 cells. The cells were cultured in 2× yeast-extract tryptone media containing carbenicillin to O.D.600 0.8-1.0, and induced with 1 mM Isopropyl β-D-1-thiogalactopyranoside (IPTG) for 3 h at 37° C. The cells were then harvested by centrifugation, and the cell pellets flash-frozen using liquid Nitrogen. Subsequently, the cell pellets were thawed, and re-suspended using a lysis buffer containing 50 mM Tris, 150 mM NaCl, 1% Triton X-100, 1 mg/ml lysozyme, 2 mM MgCl₂, and 10 units of benzonase, and incubated for 1 hour at 4° C. The lysates were cleared by centrifugation and applied to rProtein A-Sepharose columns, then washed with 10 column volumes of 50 mM Tris, 150 mM NaCl, pH 7.4. The Fab proteins were eluted with 100 mM phosphoric acid buffer, pH 2.5 (50 mM NaH₂PO₄, 140 mM NaCl, 100 mM H₃PO₄) into a neutralizing buffer consisting of 1 M Tris, pH 8.0. The eluted Fab proteins were buffer exchanged into PBS and concentrated using an Amicon-Ultra centrifugal filter unit. Subsequently, the Fabs were characterized for purity by SDS-PAGE gel chromatography and concentration by spectrophotometry at absorbance at 280 nm (FIGS. 7A-7B and data not shown).

For production of full-length IgG Abs, plasmids were DNA synthesized or subcloned from Fab-phage where DNA sequences of variable domains were subcloned into two mammalian expression vectors for heavy chain and light chain expression. The plasmids were co-transfected into Expi293 cells using the FuGENE® 6 Transfection Reagent, according to the manufacturer's instructions. The cell culture media was harvested 5-6 days following transfection, and applied to an rProtein A affinity column. IgG proteins were eluted with 25 mM H₃PO₄, pH 2.8, 100 mM NaCl and neutralized with 0.5 M Na₃PO₄ pH 8.6. Eluted fractions of interest were combined, concentrated, and dialyzed into PBS, pH 7.4.

Fab-Phage ELISAs and Cellular ELISAs

After four rounds of cellular selection, phages were produced from individual clones grown in a 96-well format. More specifically, colonies of E. coli Omnimax harboring phagemids were inoculated directly into 450 μl of 2YT broth supplemented with carbenicillin and M13-KO7 helper phage; the cultures were grown overnight at 37° C. in a 96 well format. Culture supernatants containing Fab-phage were diluted two-fold in PBS buffer supplemented with 1% BSA and incubated for 15 minutes at room temperature. For ELISAs Fab-phage were incubated against target antigens previously incubated overnight at 4° C. on Maxisorp 384-well plates. The plates were washed utilizing PBS+0.05% Tween and presented with anti-M13 HRP conjugated Ab for 30 minutes at room temperature and then assayed by colorimetric read-out. For cellular ELISA Fab-phage mixtures were added to the cellular media to plates coated with parental versus antigen expressing adherent cells at a 95-100% confluence and incubated for 30 minutes at room temperature. Following, the plates were gently washed with PBS, the cells were fixed using 4% paraformaldehyde, and then washed again with PBS. The cells were incubated for 30 minutes with horseradish peroxidase/anti-M13 Ab conjugate in PBS buffer supplemented with 1% BSA. The plates were washed, developed with TMB Microwell Peroxidase Substrate Kit, quenched with 1.0 M phosphoric acid, and absorbance determined at a wavelength of 450 nm. Positive binding clones were determined by setting a threshold of 1.5-fold or greater signal of antigen expressing cells over parental cells. All positive clones were then subjected to Sanger DNA sequence analysis.

Bio-Layer Interferometry

Affinity estimation and specificity measurements were performed using Octet HTX instrument. Recombinant integrin αv heterodimer (e.g., integrin-αvβ1) was captured on AHQ BLI sensors (18-5001, PALL), and then transferred into 100 nM antibody solutions in assay buffer (PBS, 1% BSA, 0.05% Tween20) and association was monitored for 300 seconds. Sensors were then transferred into assay buffer and dissociation was monitored for an additional 300 seconds. End-points response values were tabulated and curves fitted to Langmuir model using Octet Data Analysis Software version 9.0. Shake speed was 1000 rpm and temperate 25° C. for all assay steps.

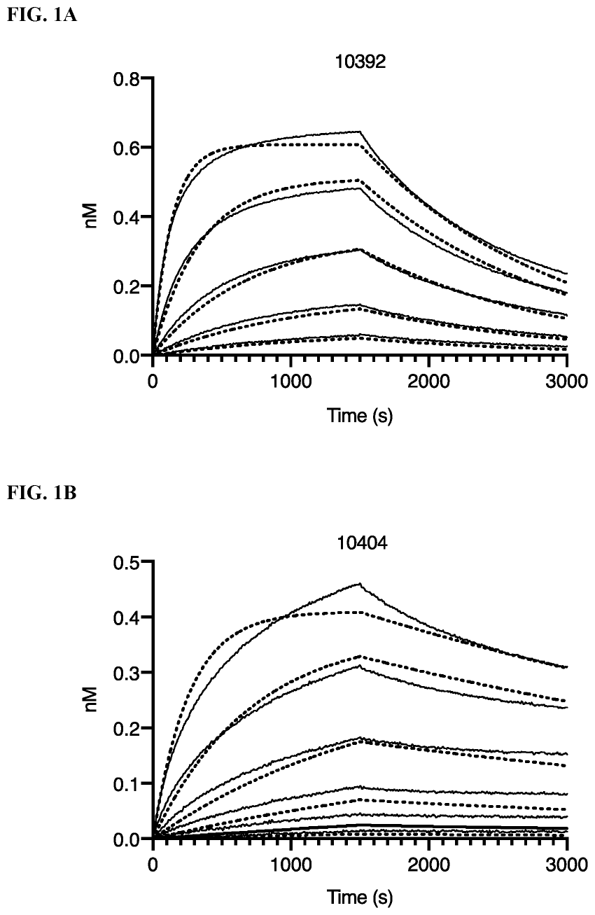

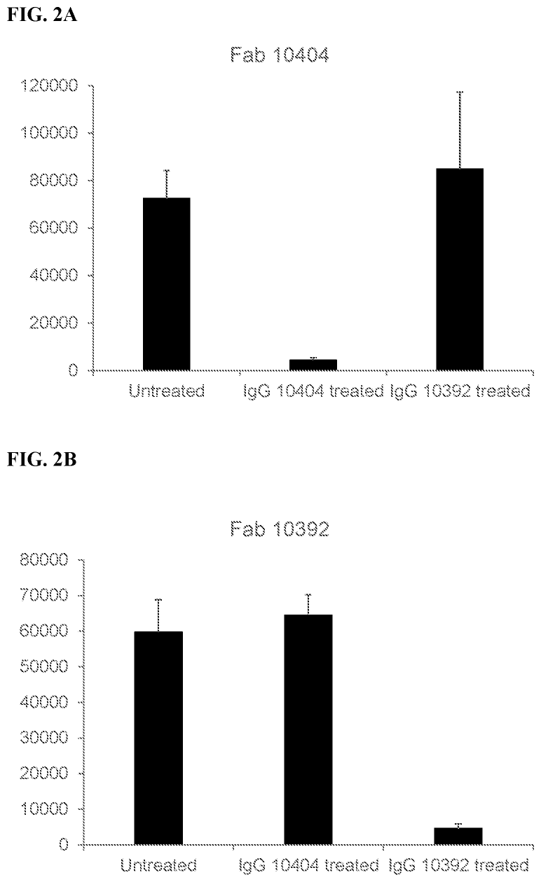

Affinity value titration graphs for IgG clones 10404 and 10392 are shown in FIGS. 1A and 1i, respectively. FIGS. 2A-2B provide a summary of epitope binning of the different Abs by labeling integrin αv heterodimer (e.g., integrin-αvβ1) CHO cells with IgG forms of each clone, and followed by Fab binding measurements. FIG. 9 provides a summary of integrin αv heterodimer (e.g., integrin-αvβ1) CHO cellular adhesion for LAP-TGFβ in the presence of different clone IgGs.

Flow-Cytometry Validations and Cellular Binding Titrations

Adherent cells were brought into suspension using PBS supplemented with 10 mM ethylenediaminetetraacetic acid (EDTA). The cells were washed with PBS, resuspended in PBS supplemented with 1% BSA, and incubated for 15 minutes at 4° C. The cells were labelled with 50 μl of Fab-phage culture supernatant, or 500 nM Fab, or IgG for 30 minutes at 4° C., then washed with PBS and resuspended in PBS supplemented with 1% BSA. Next, the cells were labelled with anti-Fab (for Fab-phage), or anti-Flag (for Fabs) or anti-Fc (for IgGs) conjugated Alexa-488 secondary Ab according to manufacturer's instructions. Data were collected using a CytoFLEX-S flow-cytometer using a 488-nm laser with 530/25 nm filter settings. The cells were analyzed in PBS, and all acquired live events were greater than 10,000 cells per sample. Quantitation analysis was carried out using FlowJo v10.2 Software. For Ab cellular titration analysis, the Abs were added to antigen positive cells in duplicate samples from a range of 500 pM to 1 μM. The mean fluorescence signal values were subtracted from the control antigen negative cells signals, and EC50 determined using Graph-pad Prism, where x is the Fab concentration:

$$Y = {Y}_{max} + \frac{{Y}_{min} - {Y}_{max}}{1 + {( \frac{EC 50}{X} )}^{HillSlope}}$$

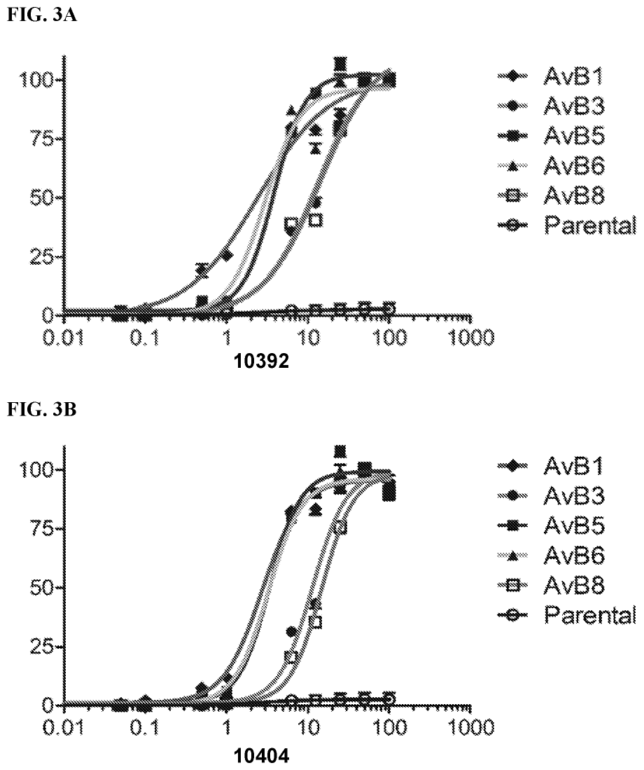

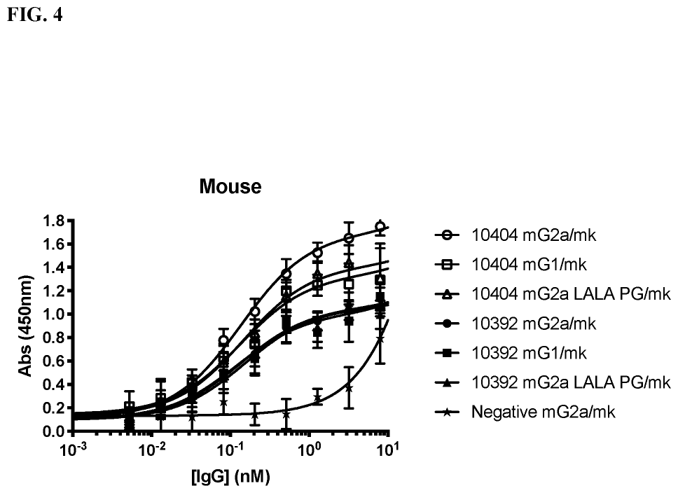

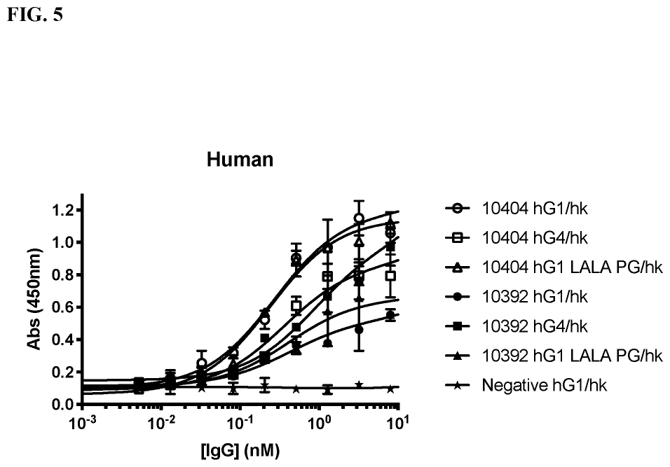

Affinity validations of the identified Ab clones for different cellular integrins are shown in FIGS. 3A-3B and Table 2. Table 2 shows unique clone IDs and corresponding EC50 affinity values for each of the different integrin CHO cell lines. Affinity validations of the different modalities of Ab clones for recombinant human and mouse integrin αv heterodimer (e.g., integrin-αvβ1) are shown in FIGS. 4 and 5, respectively, with specific values provided in Tables 3 and 4.

TABLE 2
Affinity Validations - Cellular Integrins

| EC50 (nM) | αvβ1 | αvβ3 | αvβ5 | αvβ6 | αvβ8 |
|---|---|---|---|---|---|
| 10392 | 2.72 | 14.84 | 3.40 | 3.26 | 11.45 |
| 10404 | 2.43 | 13.09 | 2.45 | 2.96 | 14.27 |

TABLE 3
Affinity Validations - Recombinant Human Integrins

| | 10404 | 10404 | 10404 hG1 | 10392 | 10392 | 10392 hG1 |
|---|---|---|---|---|---|---|
| | hG1/hk | hG4/hk | LALA PG/hk | hG1/hk | hG4/hk | LALA PG/hk |
| One site -- Total | | | | | | |
| Best-fit values | | | | | | |
| Bmax | 1.09 | 0.7817 | 1.09 | 0.4119 | 0.8498 | 0.5548 |
| Kd (nM) | 0.2705 | 0.3584 | 0.2437 | 0.4207 | 0.842 | 0.4112 |
| NS | 0.002864 | 0.004551 | −0.000281 | 0.00458 | 0.0101 | 0.0008385 |
| Background | 0.1011 | 0.08922 | 0.06215 | 0.1142 | 0.1474 | 0.1039 |
| Std. Error | | | | | | |
| Bmax | 0.04875 | 0.04479 | 0.04263 | 0.02603 | 0.05716 | 0.06411 |
| Kd | 0.05209 | 0.08571 | 0.04138 | 0.1086 | 0.203 | 0.1947 |
| NS | 0.001524 | 0.001385 | 0.001335 | 0.0007963 | 0.001611 | 0.001964 |
| Background | 0.03057 | 0.02557 | 0.0276 | 0.014 | 0.02257 | 0.03479 |
| 95% CI (profile | | | | | | |
| likelihood) | | | | | | |
| Bmax | 0.9915 to | 0.693 to | 1.004 to | 0.359 to | 0.7326 to | 0.4289 to |
| | 1.19 | 0.8729 | 1.177 | 0.4704 | 0.9955 | 0.6926 |
| Kd | 0.1848 to | 0.233 to | 0.1762 to | 0.2291 to | 0.4596 to | 0.1493 to |
| | 0.3958 | 0.5494 | 0.3377 | 0.8085 | 1.581 | 1.04 |
| NS | −0.0002564 to | 0.00178 to | −0.002989 to | 0.002767 to | 0.006188 to | −0.003312 to |
| | 0.005908 | 0.007244 | 0.002374 | 0.006234 | 0.01352 | 0.0047 |
| Background | 0.0384 to | 0.03814 to | 0.006268 to | 0.08343 to | 0.09737 to | 0.02984 to |
| | 0.1618 | 0.1388 | 0.1166 | 0.1438 | 0.1955 | 0.172 |
| Goodness of Fit | | | | | | |
| Degrees of | 32 | 32 | 32 | 32 | 32 | 32 |
| Freedom | | | | | | |
| R square | 0.9656 | 0.9564 | 0.9693 | 0.9608 | 0.9721 | 0.826 |
| Absolute Sum of | 0.272 | 0.2069 | 0.2146 | 0.06492 | 0.2012 | 0.3982 |
| Squares | | | | | | |
| Sy.x | 0.0922 | 0.0804 | 0.08189 | 0.04504 | 0.07928 | 0.1115 |
| Number of | | | | | | |
| points | | | | | | |
| # of X values | 36 | 36 | 36 | 36 | 36 | 36 |
| # Y values | 36 | 36 | 36 | 36 | 36 | 36 |
| analyzed | | | | | | |

TABLE 4
Affinity Validations - Recombinant Mouse Integrins

| | 10404 | 10404 | 10404 hG1 | 10392 | 10392 | 10392 hG1 |
|---|---|---|---|---|---|---|
| | hG1/hk | hG4/hk | LALA PG/hk | hG1/hk | hG4/hk | LALA PG/hk |
| One site -- Total | | | | | | |
| Best-fit values | | | | | | |
| Bmax | 1.09 | 0.7817 | 1.09 | 0.4119 | 0.8498 | 0.5548 |
| Kd (nM) | 0.2705 | 0.3584 | 0.2437 | 0.4207 | 0.842 | 0.4112 |
| NS | 0.002864 | 0.004551 | −0.000281 | 0.00458 | 0.0101 | 0.0008385 |
| Background | 0.1011 | 0.08922 | 0.06215 | 0.1142 | 0.1474 | 0.1039 |
| Std. Error | | | | | | |
| Bmax | 0.04875 | 0.04479 | 0.04263 | 0.02603 | 0.05716 | 0.06411 |
| Kd | 0.05209 | 0.08571 | 0.04138 | 0.1086 | 0.203 | 0.1947 |
| NS | 0.001524 | 0.001385 | 0.001335 | 0.0007963 | 0.001611 | 0.001964 |
| Background | 0.03057 | 0.02557 | 0.0276 | 0.014 | 0.02257 | 0.03479 |
| 95% CI (profile | | | | | | |
| likelihood) | | | | | | |
| Bmax | 0.9915 to | 0.693 to | 1.004 to | 0.359 to | 0.7326 to | 0.4289 to |
| | 1.19 | 0.8729 | 1.177 | 0.4704 | 0.9955 | 0.6926 |
| Kd | 0.1848 to | 0.233 to | 0.1762 to | 0.2291 to | 0.4596 to | 0.1493 to |
| | 0.3958 | 0.5494 | 0.3377 | 0.8085 | 1.581 | 1.04 |
| NS | −0.0002564 to | 0.00178 to | −0.002989 to | 0.002767 to | 0.006188 to | −0.003312 to |
| | 0.005908 | 0.007244 | 0.002374 | 0.006234 | 0.01352 | 0.0047 |
| Background | 0.0384 to | 0.03814 to | 0.006268 to | 0.08343 to | 0.09737 to | 0.02984 to |
| | 0.1618 | 0.1388 | 0.1166 | 0.1438 | 0.1955 | 0.172 |
| Goodness of Fit | | | | | | |
| Degrees of | 32 | 32 | 32 | 32 | 32 | 32 |
| Freedom | | | | | | |
| R square | 0.9656 | 0.9564 | 0.9693 | 0.9608 | 0.9721 | 0.826 |
| Absolute Sum of | 0.272 | 0.2069 | 0.2146 | 0.06492 | 0.2012 | 0.3982 |
| Squares | | | | | | |
| Sy.x | 0.0922 | 0.0804 | 0.08189 | 0.04504 | 0.07928 | 0.1115 |
| Number of | | | | | | |
| points | | | | | | |
| # of X values | 36 | 36 | 36 | 36 | 36 | 36 |
| # Y values | 36 | 36 | 36 | 36 | 36 | 36 |
| analyzed | | | | | | |

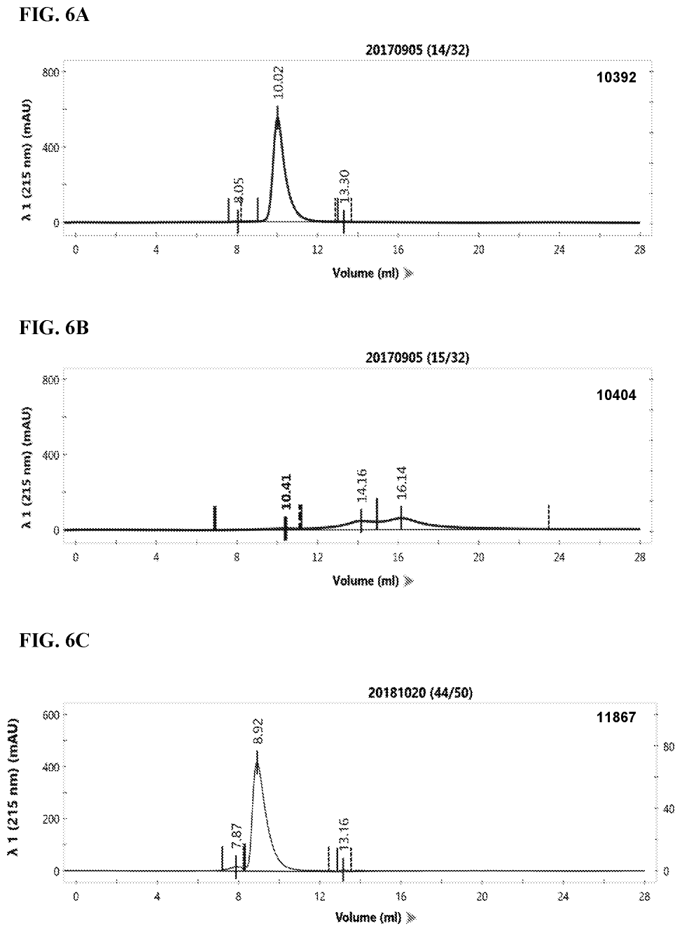

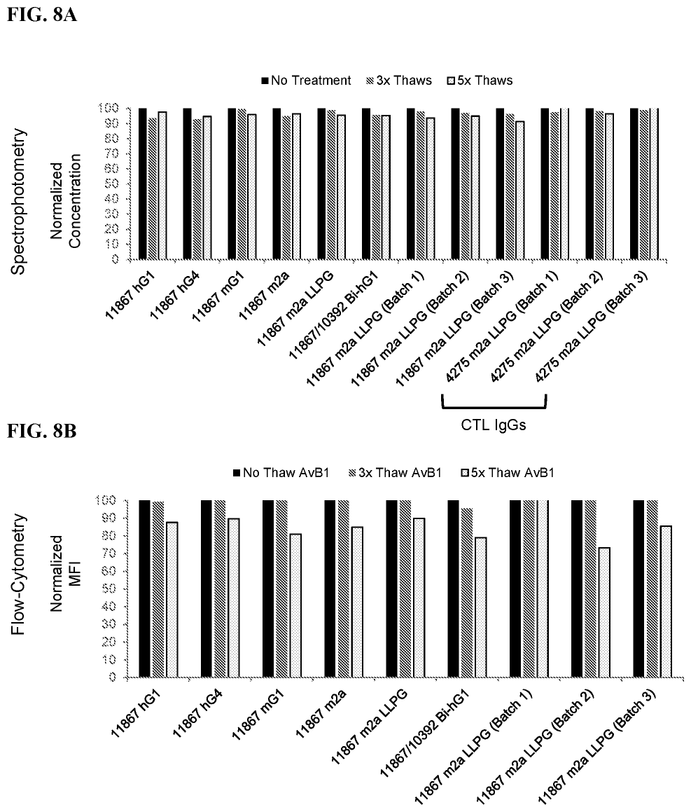

Size exclusion chromatography was used to further characterize the various IgG clones (FIGS. 6A-6C). The mobile phase used was PBS, pH 7.4 with injection of 50 ug IgG at 1 g/l at flow rate of 0.5 ml/min. Freeze-thaw analysis shows stability after at least three thaws (FIGS. 8A-8B).

Cell Attachment Assay

A 96-well plate was coated with 10 ug/ml of collagen-I ligand in PBS and incubated overnight at 4° C. The next day, each well was blocked using 2% heat-inactivated BSA in PBS for 1 hour at 37° C. Next, parental or integrin αv heterodimer (e.g., integrin-αvβ1) expressing CHO cells were brought to suspension utilizing TrypLE Express cell dissociation reagent, and then washed two times in PBS (with calcium and magnesium). These cells were then presented with Fab protein or IgG and incubated for 30 minutes at 37° C. The 96-well plate containing BSA was washed three times using PBS (with calcium and magnesium). The cells were then plated in triplicate onto the 96-well plate and incubated for 60 minutes at 37° C., and then gently washed three times using PBS (with calcium and magnesium). Cellular confluence counts were determined utilizing an IncuCyte S2 microscope and analyzed utilizing IncuCyte S2 software.

Cellular Proliferation and Migration Assays

Cells were plated using 100 μl growth media in 96-well tissue culture treated plates and grown in tissue culture incubator. Triplicate samples were treated with 200 nM IgG and measured by phase-contrast microscopy every 2 hours with four images per well using IncuCyte S2 microscope. All samples were analyzed using Incucyte S2 software for cellular confluence percentages in proliferation assays, and wound healing in migration assays.

NGS Samples Preparation and Illumina Read-Out

PCR amplicons were generated utilizing expanded fourth round Fab-phage pools where the forward and reverse primers flanked the Fab region between the complementary determining region (CDR) VL3 and VH3. Both, forward and reverser primers, included a 24 base pair length template annealing regions followed by a 6-8 base pair length unique nucleotide “barcode” identifiers and the Illumina universal adapter tag (PE1 for the reverse and PE2 for the forward primer). Amplicons were isolated by gel electrophoresis followed by agarose gel extraction according to manufacturer's instructions. Sample concentrations were determined in duplicates utilizing a micro-volume spectrophotometer. All the isolated amplicons were normalized, pooled, and sequenced using an Illumina HiSeq 2500 instrument with paired-end 300 cycles setting. In order to cover the entire CDRs, in addition to the PE1 and PE2 Illumina primers, a custom primer annealing prior the VH2 and VH1 regions was also included.

Scoring Separation Between Distributions of Frequencies in Positive and Negative Selections

To score the separation between the two distributions of frequencies of the motifs in the positive and the negative selection pools we utilized the Welch-Satterthwaite version of the t-test in conjunction with the rank transformation. This approach has the advantage to counteract simultaneously the undesirable effects of both non-normality and unequal variances in our distributions of frequencies. First, the two distributions of frequencies are combined and arranged in ascending order, with tied frequencies receiving a rank equal to the average of their positions. Given the two distributions of ranks x_{P} and x_{N} that correspond to the frequencies in the positive and the negative selection pools, and x̅_{P} and x̅_{N} are the means, s_{P} and s_{N} are the standard deviations, and n_{P} and n_{P} are the sizes, all respectively. We calculate the p-value to evaluate the statistical significance of the t-score following the procedure described below

We first calculate the t-score by:

$$t = \frac{{\overline{x}}_{P} - {\overline{x}}_{N}}{\sqrt{\frac{{s}_{P}^{2}}{{n}_{P}} + \frac{{s}_{N}^{2}}{{n}_{N}}}}$$

We then calculate the degree of freedom by:

$$f = \frac{{( \frac{{s}_{P}^{2}}{{n}_{P}} + \frac{{s}_{N}^{2}}{{n}_{N}} )}^{2}}{\frac{{( \frac{{s}_{P}^{2}}{{n}_{P}} )}^{2}}{{n}_{P} - 1} + \frac{{( \frac{{s}_{N}^{2}}{{n}_{N}} )}^{2}}{{n}_{N} - 1}}$$

We finally calculate the p-value by:

$$p = \frac{1}{\sqrt{f π}} \frac{Γ ( 1 \frac{f + 1}{2} )}{Γ ( \frac{f}{2} )} {\int }_{- ∞}^{t} \frac{1}{{( 1 + \frac{{t}^{2}}{f} )}^{\frac{f + 1}{2}}} dt$$

Where t is the t-score, f is the degree of freedom, Γ(⋅) is the Gamma function, and p is the probability that a single observation from the t distribution with f degrees of freedom will fall in the interval [−∞, t]. In other terms, the p is the probability to have by chance any t-score that is equal or below to the t-score t. Thus, the lower is the p-value p, the higher is the significance of the t-score t, and consequently the higher is the separation between the two distributions of frequencies in the positive and the negative selections. We filtered the p-values using a stringent cut-off of [symbol] 10 [symbol] \hat{ }(−10) to identify highly specific Ab clones. To complement the p-values, we calculated Cohen's d effect size coefficient to evaluate the difference between the means of the frequencies in the positive and the negative selections, and we kept p-values with huge effect size (d≥2).

$$d = \frac{{\overline{x}}_{P} - {\overline{x}}_{N}}{\sqrt{\frac{( {n}_{P} - 1 ) {s}_{P}^{2} + ( {n}_{N} - 1 ) {s}_{N}^{2}}{{n}_{P} + {n}_{N} - 2}}}$$

Enumeration of Consensus Motifs in the CDR Sequences

Consensus motifs are utilized to represent the linear information that is shared among groups of sequences. While certain positions in the motifs are defined (e.g. “P” or “R” in “P..R”), others do not and are called wildcards (e.g. “.” in “P..R”). We utilize here consensus motifs to explore the linear information that is shared between the CDR sequences of the candidate Ab and the rest of the Abs in the positive and the negative selections. To this end, we adapted the algorithm DALEL that was first developed to explore the linear information in proteins. To avoid the explosion of the number of motifs, we restricted the number of allowed wildcards in each motif to 55% of its length. In addition, we considered only motifs with wildcards matching different amino acids in the matching sequences in the positive selection, e.g. wildcard in motif “P.R” matching amino acids “Y” and “S” in matching sequences “PYR” and “PSR”. Finally, we restricted the number of motifs to the most representatives by limiting the minimum number of sequences in the positive selection matching every motif to a 100.

### Example 2: Generation and Characterization of Bispecific Antibodies Targeting Integrin αv Heterodimer (e.g., Integrin-αvβ1)

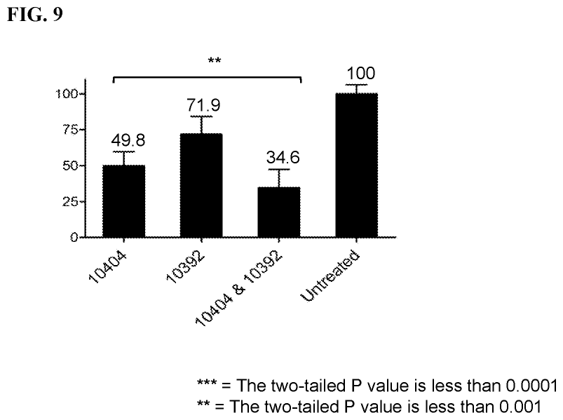

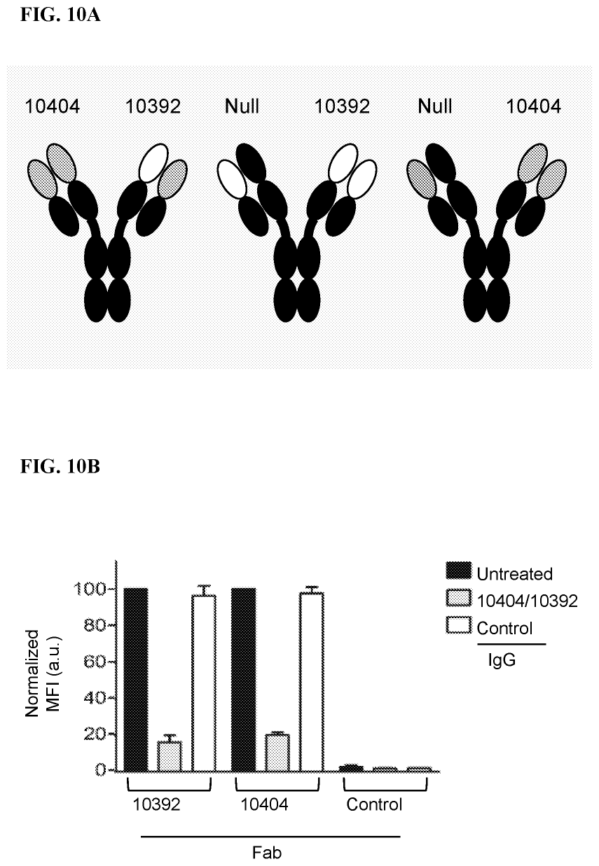

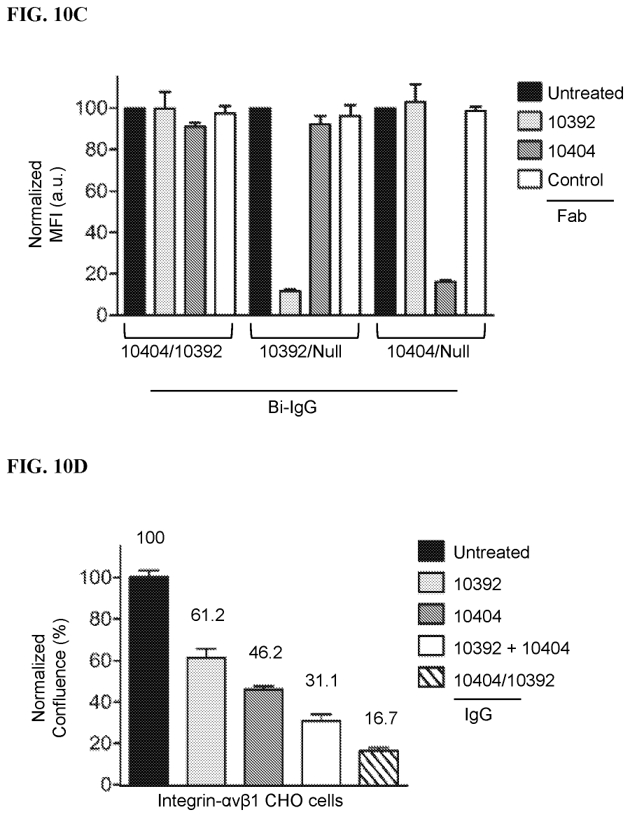

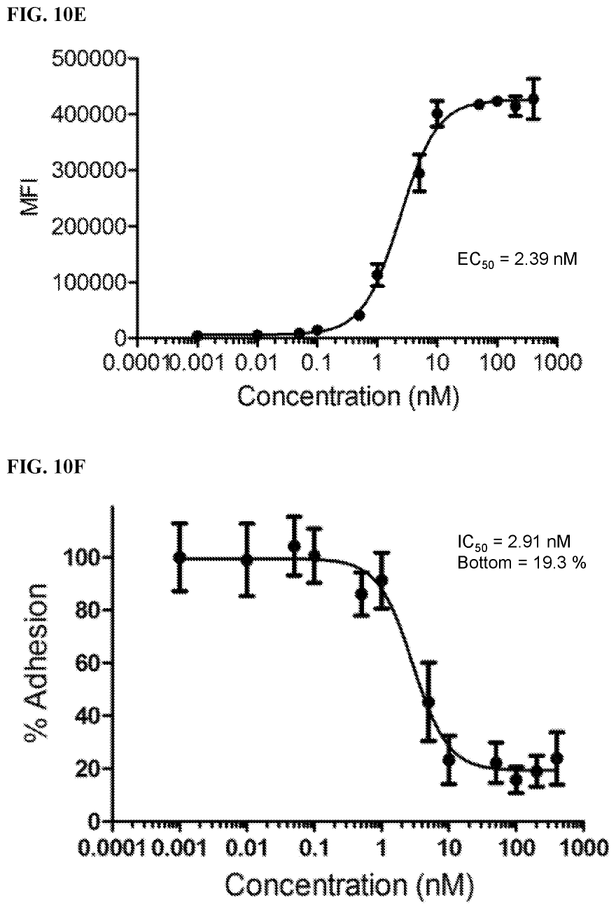

To further target integrin αv heterodimer (e.g., integrin-αvβ1), two bispecific antibodies were generated using components of various IgG monospecific antibodies described in Example 1. In particular, IgG clones 10404, 10392, and 11867 heavy chains and light chains were exchanged, and functional properties and cellular binding titrations were assessed. FIG. 10A provides illustrations of different bi-specific IgGs, where null indicates a non-selective (random CDRs) bi-IgG arm. Fab binding activity for integrin αv heterodimer (e.g., integrin-αvβ1) CHO cells in the presence of bi-specific IgG 10404/10392 is shown in FIG. 10B. The bi-specific IgG 10404/10392 comprises (i) a heavy chain 10392 sequence (SEQ ID NO: 31), comprising modifications relative to SEQ ID NO: 1 that introduce a knob motif, (ii) a heavy chain 10404 sequence (SEQ ID NO: 34), comprising modifications relative to SEQ ID NO: 11 that introduce a hole motif, and (iii) two 10404 light chain sequences (SEQ ID NO: 16). The heavy chain knob and hole sequences facilitate heterodimerization of the two heavy chains.

Binding affinity of 10404-10392, 10392/null, and 10404/null bi-specific IgGs for integrin αv heterodimer (e.g., integrin-αvβ1) CHO cells in the presence of different Fab clones is shown in FIG. 10C. integrin αv heterodimer (e.g., integrin-αvβ1) CHO cellular adhesion for LAP-TGFβ in the presence of different clone IgGs is shown in FIG. 10D, and FIG. 10E provides a titration graph for integrin αv heterodimer (e.g., integrin-αvβ1) CHO cells by flow-cytometry measurements. Inhibitory titration of integrin αv heterodimer (e.g., integrin-αvβ1) CHO cellular for adhesion for LAP-TGFβ is shown in FIG. 10E.

The inhibitory properties of the 10404/10392 bispecific antibody were then compared with those of the 10404 and 10392 monospecific antibodies (FIGS. 11A-11F).

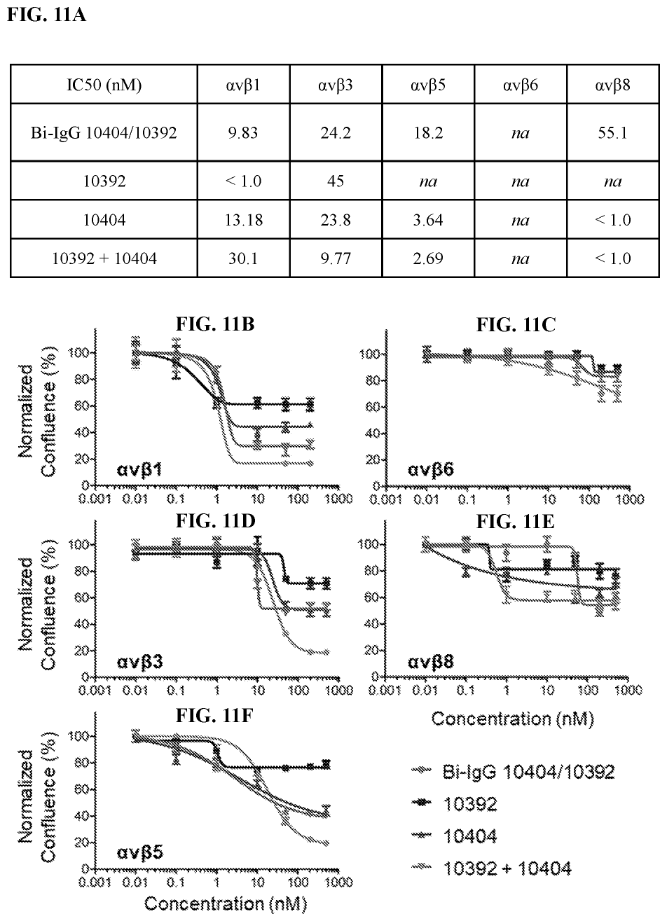

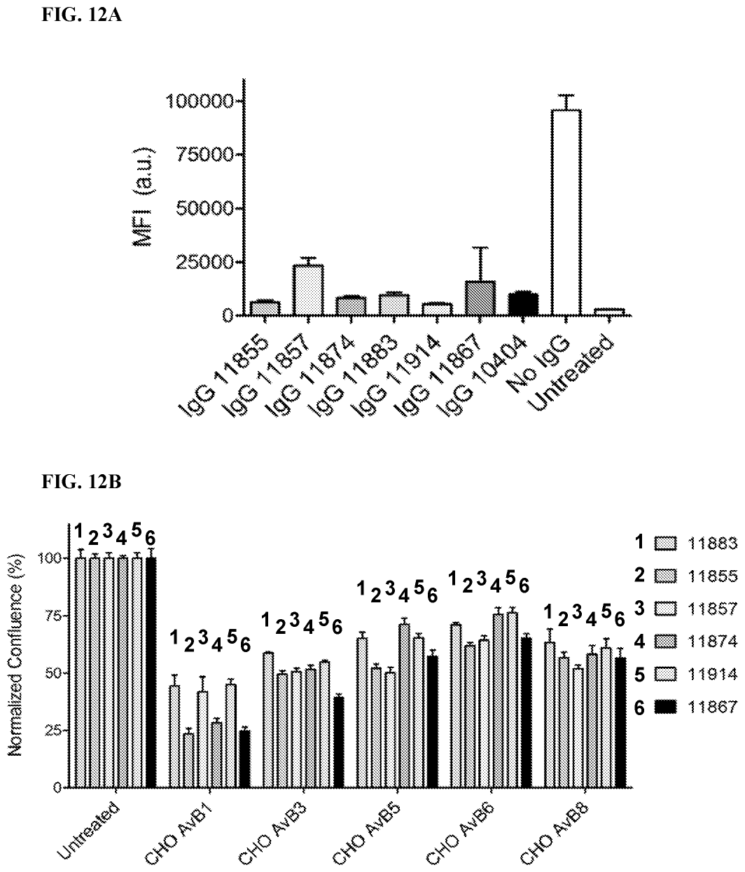

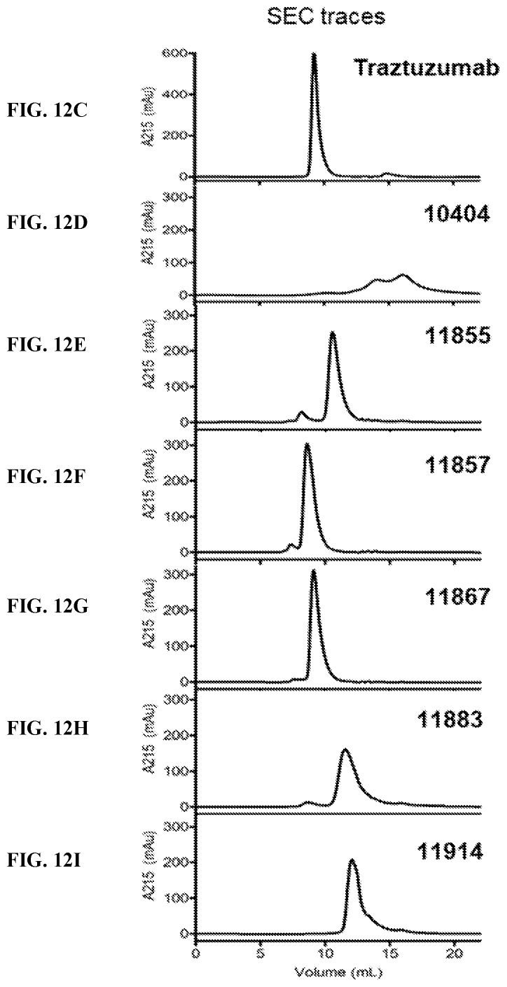

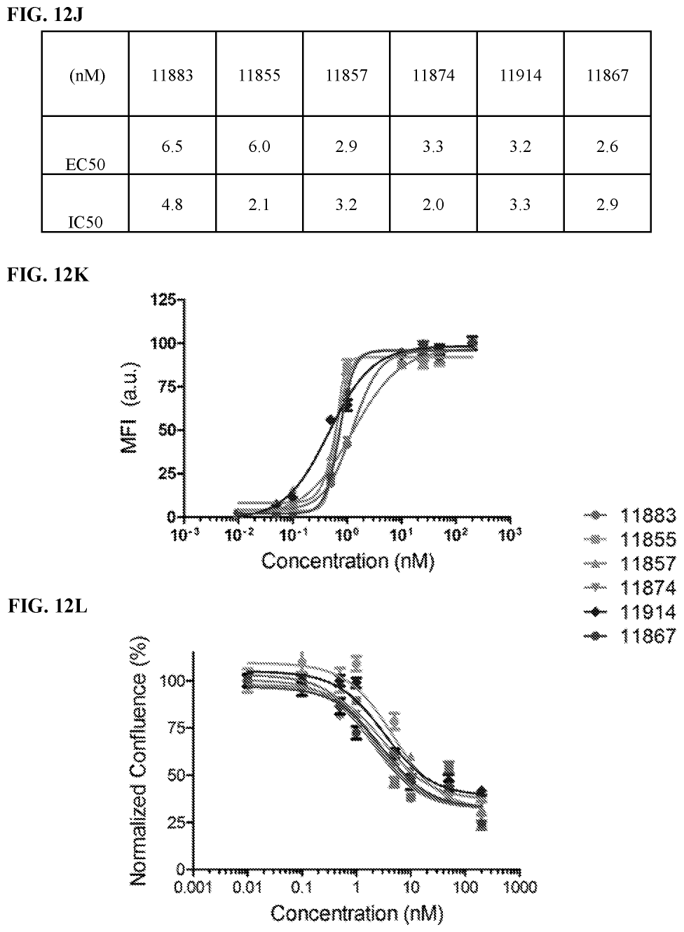

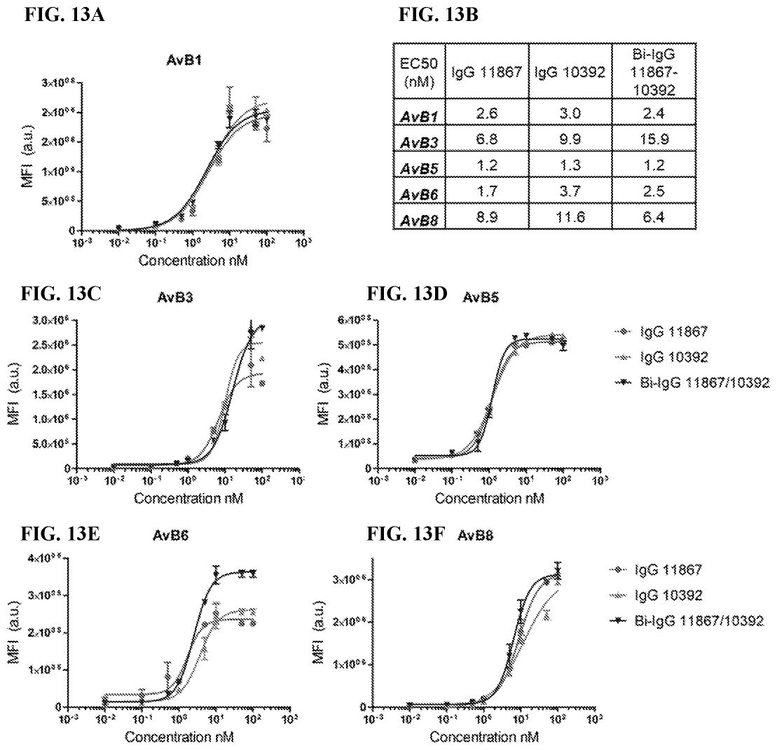

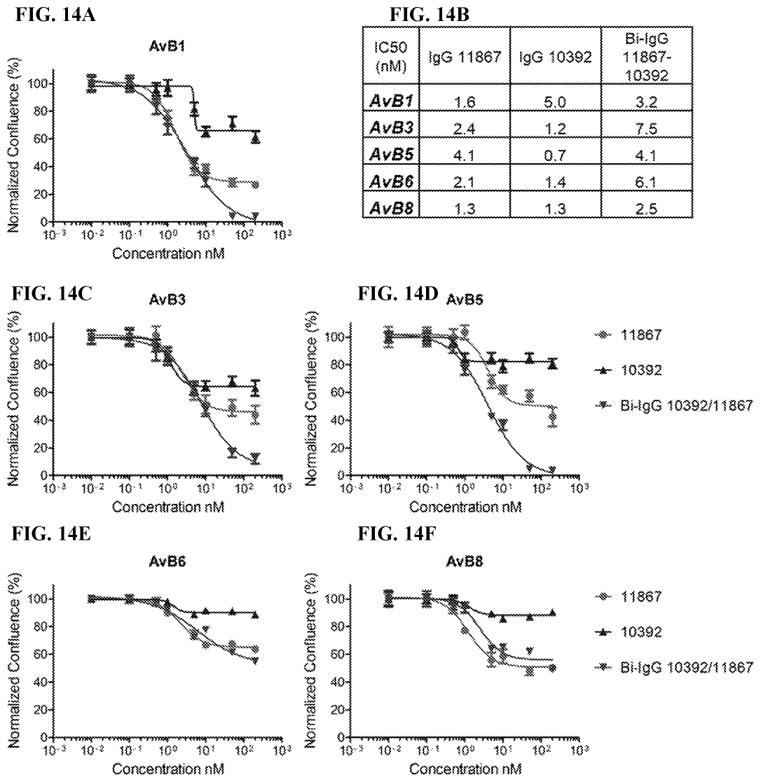

Additional variants of the 10404 clone were developed and characterized (FIGS. 12A-12L). IgG clone 11867 was identified as having improved properties, and a second bispecific antibody was generated comprising (i) a heavy chain 10392 sequence (SEQ ID NO: 31), comprising modifications relative to SEQ ID NO: 1 that introduce a knob motif, (ii) a heavy chain 11867 sequence (SEQ ID NO: 37), comprising modifications relative to SEQ ID NO: 21 that introduce a hole motif, and (iii) two 11867 light chain sequences (SEQ ID NO: 26). The inhibitory properties of the 11867/10392 bispecific antibody were then compared with those of the 11867 and 10392 monospecific antibodies (FIGS. 13A-13F and 14A-14F).

### Example 3: In Vitro Inhibition of Tumor Cell Proliferation

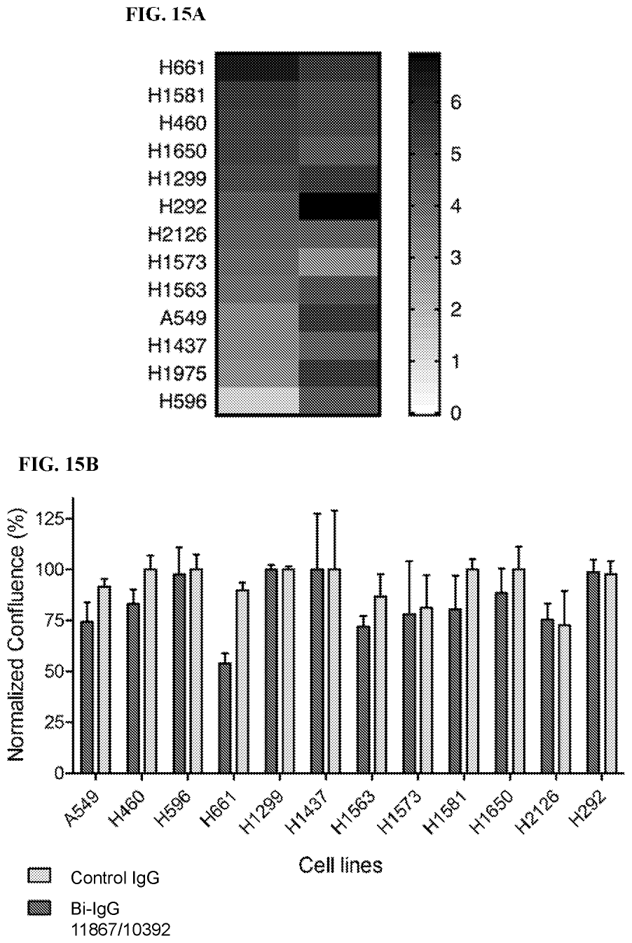

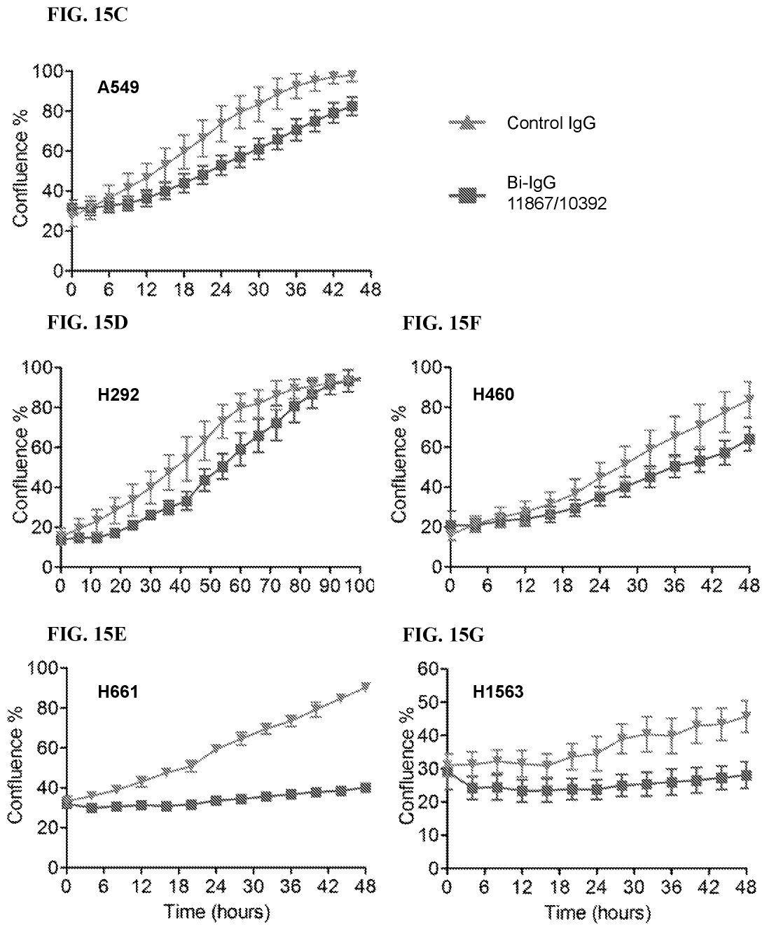

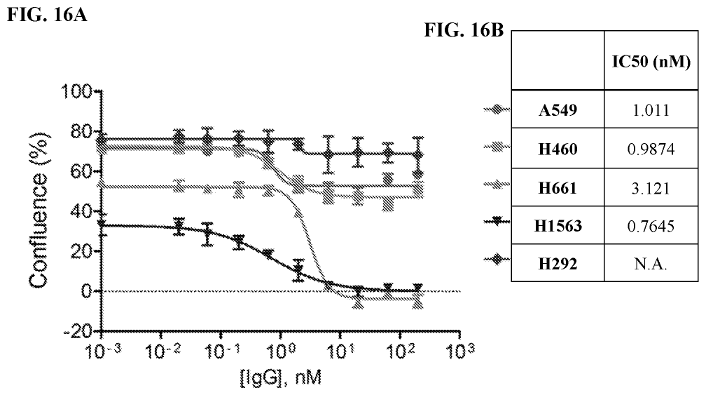

The bispecific IgG 11867/10392 antibody was then assayed for the ability to inhibit tumor cell proliferation in vitro. Various lung cancer cell lines, including A549, H292, H661, H460, and H1563, were cultured in the presence of bispecific IgG 11867/10392 or a control IgG and assayed for cell confluence over 48 hours (FIGS. 15B-15G). Tumor cells cultured in the presence of bispecific IgG 11867/10392 showed decreased proliferation in several cell types (FIG. 16A), suggesting that bispecific IgG 11867/10392 is capable of inhibiting tumor cell proliferation.

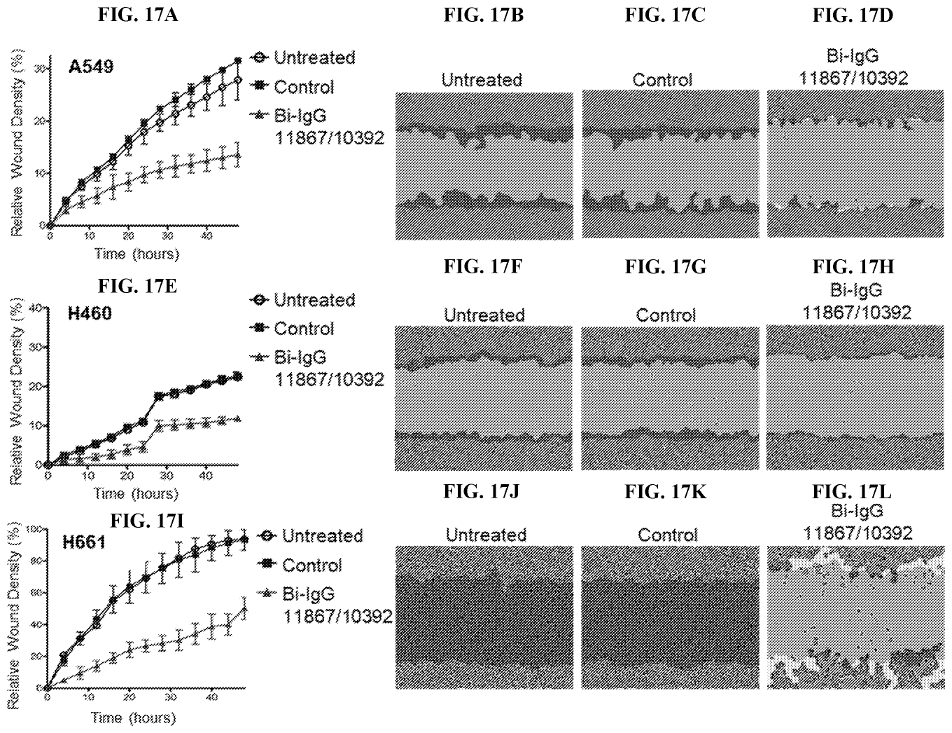

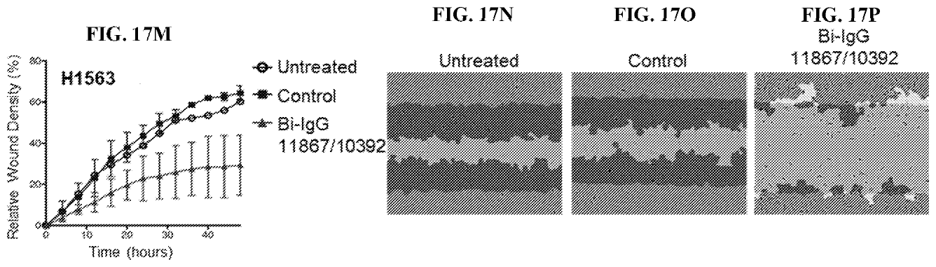

As integrin αv heterodimer (e.g., integrin-αvβ1) plays a role in cell migration, the ability of bispecific IgG 11867/10392 to inhibit tumor cell migration was then assayed. Wound healing assays using lung tumor cell lines A549, H460, H661, and H1563 showed decreased relative wound density when cells were cultured in the presence of bispecific IgG 11867/10392 as compared to untreated cells and control cells (FIGS. 17A-17P).

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

What is claimed:

1. A bispecific antibody or an antigen-binding portion thereof that specifically binds an integrin-αv heterodimer and inhibits integrin-αv-mediated activation of TGFβ.

2. The bispecific antibody or antigen-binding portion thereof of claim 1, comprising at least a first paratope and a second paratope, wherein the first paratope binds a first epitope on an integrin αvβ1 heterodimer.

3. The bispecific antibody or antigen-binding portion thereof of claim 2, wherein the second paratope binds a second epitope on the integrin αvβ1 heterodimer.

4. The bispecific antibody or antigen-binding portion thereof of claim 3, wherein the first epitope and the second epitope are not the same.

5. The bispecific antibody or antigen-binding portion thereof of any one of claims 1 to 4, comprising a first heavy chain, a first light chain, a second heavy chain, and second light chain.

6. The bispecific antibody or antigen-binding portion thereof of claim 5, wherein the first heavy chain and the second heavy chain are different.

7. The bispecific antibody or antigen-binding portion thereof of claim 5 or 6, wherein the first heavy chain and the second heavy chain are different, and wherein the first light chain and the second light chain are the same.

8. The bispecific antibody or antigen-binding portion thereof of any one of claims 5 to 7, wherein the first heavy chain comprises a first variable heavy region (“VH1”), comprising a variable heavy complementarity determining region (VH1-CDR) 1, a VH1-CDR2, and a VH1-CDR3; wherein the VH1-CDR3 comprises an amino acid sequence selected from SEQ ID NOs: 5, 15, and 25.

9. The bispecific antibody or antigen-binding portion thereof of claim 8, wherein the VH1-CDR2 comprises an amino acid sequence selected from SEQ ID NOs: 4, 14, and 24.

10. The bispecific antibody or antigen-binding portion thereof of claim 8 or 9, wherein the VH1-CDR1 comprises an amino acid sequence selected from SEQ ID NOs: 3, 13, and 23.

11. The bispecific antibody or antigen-binding portion thereof of any one of claims 5 to 10, wherein the first light chain comprises a first variable light region (“VL1”), comprising a VL1-CDR1, a VL1-CDR2, and a VL1-CDR3; wherein the VL1-CDR3 comprises an amino acid sequence selected from SEQ ID NOs: 10, 20, and 30.

12. The bispecific antibody or antigen-binding portion thereof of claim 11, wherein the VL1-CDR2 comprises an amino acid sequence selected from SEQ ID NOs: 9, 19, and 29.

13. The bispecific antibody or antigen-binding portion thereof of claim 11 or 12, wherein the VL1-CDR1 comprises an amino acid sequence selected from SEQ ID NOs: 8, 18, and 28.

14. The bispecific antibody or antigen-binding portion thereof of any one of claims 1 to 13, comprising:
(a) a VH1-CDR1 comprising the amino acid sequence set forth in SEQ ID NO: 3;
(b) a VH1-CDR2 comprising the amino acid sequence set forth in SEQ ID NO: 4;
(c) a VH1-CDR3 comprising the amino acid sequence set forth in SEQ ID NO: 5;
(d) a VL1-CDR1 comprising the amino acid sequence set forth in SEQ ID NO: 18;
(e) a VL1-CDR2 comprising the amino acid sequence set forth in SEQ ID NO: 19; and
(f) a VL1-CDR3 comprising the amino acid sequence set forth in SEQ ID NO: 20.

15. The bispecific antibody or antigen-binding portion thereof of any one of claims 1 to 13, comprising:
(a) a VH1-CDR1 comprising the amino acid sequence set forth in SEQ ID NO: 13;
(b) a VH1-CDR2 comprising the amino acid sequence set forth in SEQ ID NO: 14;
(c) a VH1-CDR3 comprising the amino acid sequence set forth in SEQ ID NO: 15;
(d) a VL1-CDR1 comprising the amino acid sequence set forth in SEQ ID NO: 18;
(e) a VL1-CDR2 comprising the amino acid sequence set forth in SEQ ID NO: 19; and
(f) a VL1-CDR3 comprising the amino acid sequence set forth in SEQ ID NO: 20.

16. The bispecific antibody or antigen-binding portion thereof of any one of claims 1 to 13, comprising:
(a) a VH1-CDR1 comprising the amino acid sequence set forth in SEQ ID NO: 3;
(b) a VH1-CDR2 comprising the amino acid sequence set forth in SEQ ID NO: 4;
(c) a VH1-CDR3 comprising the amino acid sequence set forth in SEQ ID NO: 5;
(d) a VL1-CDR1 comprising the amino acid sequence set forth in SEQ ID NO: 28;
(e) a VL1-CDR2 comprising the amino acid sequence set forth in SEQ ID NO: 29; and
(f) a VL1-CDR3 comprising the amino acid sequence set forth in SEQ ID NO: 30.

17. The bispecific antibody or antigen-binding portion thereof of any one of claims 1 to 13, comprising:
(a) a VH1-CDR1 comprising the amino acid sequence set forth in SEQ ID NO: 23;
(b) a VH1-CDR2 comprising the amino acid sequence set forth in SEQ ID NO: 24;
(c) a VH1-CDR3 comprising the amino acid sequence set forth in SEQ ID NO: 25;
(d) a VL1-CDR1 comprising the amino acid sequence set forth in SEQ ID NO: 28;
(e) a VL1-CDR2 comprising the amino acid sequence set forth in SEQ ID NO: 29; and
(f) a VL1-CDR3 comprising the amino acid sequence set forth in SEQ ID NO: 30.

18. The bispecific antibody or antigen-binding portion thereof of any one of claims 5 to 17, wherein the second heavy chain comprises a second variable heavy region (“VH2”), comprising a VH2-CDR1, a VH2-CDR2, and a VH2-CDR3; wherein the VH2-CDR3 comprises an amino acid sequence selected from SEQ ID NOs: 5, 15, and 25.

19. The bispecific antibody or antigen-binding portion thereof of claim 18, wherein the VH2-CDR2 comprises an amino acid sequence selected from SEQ ID NOs: 4, 14, and 24.

20. The bispecific antibody or antigen-binding portion thereof of claim 18 or 19, wherein the VH2-CDR1 comprises an amino acid sequence selected from SEQ ID NOs: 3, 13, and 23.

21. The bispecific antibody or antigen-binding portion thereof of any one of claims 18 to 20, wherein the second light chain comprises a second variable light region (“VL1”), comprising a VL2-CDR1, a VL2-CDR2, and a VL2-CDR3; wherein the VL2-CDR3 comprises an amino acid sequence selected from SEQ ID NOs: 20 and 30.

22. The bispecific antibody or antigen-binding portion thereof of claim 21, wherein the VL2-CDR2 comprises an amino acid sequence selected from SEQ ID NOs: 19 and 29.

23. The bispecific antibody or antigen-binding portion thereof of claim 21 or 22, wherein the VH2-CDR1 comprises an amino acid sequence selected from SEQ ID NOs: 18 and 28.

24. The bispecific antibody or antigen-binding portion thereof of any one of claims 1 to 23, comprising:
(a) a first variable heavy region (VH1), comprising
a. a VH1-CDR1 comprising the amino acid sequence set forth in SEQ ID NO: 3,
b. a VH1-CDR2 comprising the amino acid sequence set forth in SEQ ID NO: 4, and
c. a VH1-CDR3 comprising the amino acid sequence set forth in SEQ ID NO: 5;
(b) a first variable light region (VL1), comprising
a. a VL1-CDR1 comprising the amino acid sequence set forth in SEQ ID NO: 18,
b. a VL1-CDR2 comprising the amino acid sequence set forth in SEQ ID NO: 19, and
c. a VL1-CDR3 comprising the amino acid sequence set forth in SEQ ID NO: 20;
(c) a second variable heavy region (VH2), comprising
a. a VH2-CDR1 comprising the amino acid sequence set forth in SEQ ID NO: 13;
b. a VH2-CDR2 comprising the amino acid sequence set forth in SEQ ID NO: 14;
c. a VH2-CDR3 comprising the amino acid sequence set forth in SEQ ID NO: 15; and
(d) a second variable light region (VL2), comprising
a. a VL2-CDR1 comprising the amino acid sequence set forth in SEQ ID NO: 18;
b. a VL2-CDR2 comprising the amino acid sequence set forth in SEQ ID NO: 19; and
c. a VL2-CDR3 comprising the amino acid sequence set forth in SEQ ID NO: 20.

25. The bispecific antibody or antigen-binding portion thereof of any one of claims 1 to 23, comprising:
(a) a first variable heavy region (VH1), comprising
a. a VH1-CDR1 comprising the amino acid sequence set forth in SEQ ID NO: 3,
b. a VH1-CDR2 comprising the amino acid sequence set forth in SEQ ID NO: 4, and
c. a VH1-CDR3 comprising the amino acid sequence set forth in SEQ ID NO: 5;
(b) a first variable light region (VL1), comprising
a. a VL1-CDR1 comprising the amino acid sequence set forth in SEQ ID NO: 28,
b. a VL1-CDR2 comprising the amino acid sequence set forth in SEQ ID NO: 29, and
c. a VL1-CDR3 comprising the amino acid sequence set forth in SEQ ID NO: 30;
(c) a second variable heavy region (VH2), comprising
a. a VH2-CDR1 comprising the amino acid sequence set forth in SEQ ID NO: 23;
b. a VH2-CDR2 comprising the amino acid sequence set forth in SEQ ID NO: 24;
c. a VH2-CDR3 comprising the amino acid sequence set forth in SEQ ID NO: 25; and
(d) a second variable light region (VL2), comprising
a. a VL2-CDR1 comprising the amino acid sequence set forth in SEQ ID NO: 28;
b. a VL2-CDR2 comprising the amino acid sequence set forth in SEQ ID NO: 29; and
c. a VL2-CDR3 comprising the amino acid sequence set forth in SEQ ID NO: 30.

26. The bispecific antibody or antigen-binding portion thereof of any one of claims 1 to 25, comprising a first variable heavy region (VH1) and a first variable light region (VL1), wherein the VH1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22.

27. The bispecific antibody or antigen-binding portion thereof of claim 26, wherein the VL1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

28. The bispecific antibody or antigen-binding portion thereof of claim 26 or 27, comprising a second variable heavy region (VH2) and a second variable light region (VL2), wherein the VH2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2, 12, or 22.

29. The bispecific antibody or antigen-binding portion thereof of any one of claims 26 to 28, wherein the VL2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 17, or 27.

30. The bispecific antibody or antigen-binding portion thereof of claim 28 or 29, wherein:
a. the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2;
b. the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17;
c. the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12; and
d. the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17.

31. The bispecific antibody or antigen-binding portion thereof of any one of claims 28 to 30, wherein:
a. the VH1 comprises the amino acid sequence set forth in SEQ ID NO: 2;
b. the VL1 comprises the amino acid sequence set forth in SEQ ID NO: 17;
c. the VH2 comprises the amino acid sequence set forth in SEQ ID NO: 12; and
d. the VL2 comprises the amino acid sequence set forth in SEQ ID NO: 17.

32. The bispecific antibody or antigen-binding portion thereof of claim 28 or 29, wherein:
a. the VH1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2;
b. the VL1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27;
c. the VH2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 22; and
d. the VL2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

33. The bispecific antibody or antigen-binding portion thereof of any one of claims 28, 29, and 32, wherein:
a. the VH1 comprises the amino acid sequence set forth in SEQ ID NO: 2;
b. the VL1 comprises the amino acid sequence set forth in SEQ ID NO: 27;
c. the VH2 comprises the amino acid sequence set forth in SEQ ID NO: 22; and
d. the VL2 comprises the amino acid sequence set forth in SEQ ID NO: 27.

34. The bispecific antibody or antigen-binding portion thereof of any one of claims 1 to 33, comprising a first heavy chain (H1) and a first light chain (L1), wherein the H1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37.

35. The bispecific antibody or antigen-binding portion thereof of claim 34, wherein the L1 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

36. The bispecific antibody or antigen-binding portion thereof of claim 34 or 35, comprising a second heavy chain (H2) and a second light chain (L2), wherein the H2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 1, 11, 21, 31, 34, or 37.

37. The bispecific antibody or antigen-binding portion thereof of any one of claims 34 to 36, wherein the L2 comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 6, 16, or 26.

38. The bispecific antibody or antigen-binding portion thereof of any one of claims 5 to 37, wherein the first heavy chain is associated with the second heavy chain.

39. The bispecific antibody or antigen-binding portion thereof of any one of claims 5 to 38, wherein the first heavy chain is associated with the second heavy chain by a covalent bond.

40. The bispecific antibody or antigen-binding portion thereof of any one of claims 5 to 39, wherein each of the first heavy chain and the second heavy chain comprises an IgG constant region or an IgG constant region comprising one or more amino acid substitutions.

41. The bispecific antibody or antigen-binding portion thereof of claim 40, wherein the one or more amino acid substitutions promotes heterodimerization of the first heavy chain and the second heavy chain.

42. The bispecific antibody or antigen-binding portion thereof of any one of claims 5 to 41, wherein the first heavy chain comprises a substitution of one or more amino acids in a constant region of the first heavy chain, creating a knob; wherein the second heavy chain comprises a substitution or antigen-binding portion thereof of one or more amino acids in a constant region of the second heavy chain, creating a hole; wherein the knob of the first heavy chain associates with the hole of the second heavy chain.

43. The bispecific antibody or antigen-binding portion thereof of any one of claims 5 to 41, wherein the first heavy chain comprises a substitution of one or more amino acids in a constant region of the first heavy chain, creating a hole; wherein the second heavy chain comprises a substitution of one or more amino acids in a constant region of the second heavy chain, creating a knob; wherein the hole of the first heavy chain associates with the knob of the second heavy chain.

44. The bispecific antibody or antigen-binding portion thereof of any one of claims 34 to 43, wherein:
a. the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31;
b. the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16;
c. the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 34; and
d. the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16.

45. The bispecific antibody or antigen-binding portion thereof of any one of claims 34 to 44, wherein:
a. the H1 comprises the amino acid sequence set forth in SEQ ID NO: 31;
b. the L1 comprises the amino acid sequence set forth in SEQ ID NO: 16;
c. the H2 comprises the amino acid sequence set forth in SEQ ID NO: 34; and
d. the L2 comprises the amino acid sequence set forth in SEQ ID NO: 16.

46. The bispecific antibody or antigen-binding portion thereof of any one of claims 34 to 43, wherein:
a. the H1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 31;
b. the L1 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26;
c. the H2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 37; and
d. the L2 comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26.

47. The bispecific antibody or antigen-binding portion thereof of any one of claims 34 to 43 and 46, wherein:
a. the H1 comprises the amino acid sequence set forth in SEQ ID NO: 31;
b. the L1 comprises the amino acid sequence set forth in SEQ ID NO: 26;
c. the H2 comprises the amino acid sequence set forth in SEQ ID NO: 37; and
d. the L2 comprises the amino acid sequence set forth in SEQ ID NO: 26.

48. The bispecific antibody or antigen-binding portion thereof of any one of claims 1 to 47, wherein the bispecific antibody has one or more properties selected from the group consisting of:
(a) the bispecific antibody inhibits binding of integrin-αvβ1 to LAP-TGFβ1;
(b) the bispecific antibody is capable of binding an integrin αv selected from αvβ1, αvβ3, αvβ5, αvβ6, αvβ8, and any combination thereof,
(c) the bispecific antibody inhibits cell adhesion;
(d) the bispecific antibody inhibits tumor growth and/or metastasis;
(e) the bispecific antibody increases progression-free survival;
(f) the bispecific antibody increases overall survival; and
(g) any combination thereof.

49. The bispecific antibody or antigen-binding portion thereof of any one of claims 1 to 48, which is capable of (i) inhibiting the binding of integrin-αvβ1 to LAP-TGFβ1 and (ii) inhibiting cell adhesion.

50. The bispecific antibody or antigen-binding portion thereof of any one of claims 1 to 49, which binds integrin-αv with a K_{D} of about 10⁻⁶ M or less, about 10⁻⁷ M or less, about 10⁻⁸ M or less, about 10⁻⁹ M or less, about 10⁻¹⁰ M or less, about 10⁻¹¹ M or less, or about 10⁻¹² M or less.

51. An isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein the VH-CDR3 comprises an amino acid sequence of SEQ ID NOs: 5, 15, or 25.

52. The antibody or antigen binding-portion thereof of claim 51, wherein the VH-CDR2 comprises an amino acid sequence of SEQ ID NO: 4, 14, or 24.

53. The antibody or antigen binding-portion thereof of claim 51 or 52, wherein the VH-CDR1 comprises an amino acid sequence of SEQ ID NO: 3, 13, or 23.

54. The antibody or antigen binding-portion thereof of any one of claims 51 to 53, wherein the VL-CDR3 comprises an amino acid sequence SEQ ID NO: 10, 20, or 30.

55. The antibody or antigen binding-portion thereof of claim 54, wherein the VL-CDR2 comprises an amino acid sequence of SEQ ID NO: 9, 19, or 29.

56. The antibody or antigen binding-portion thereof of claim 51 or 52, wherein the VL-CDR1 comprises an amino acid sequence of SEQ ID NOs: 8, 18, or 28.

57. An isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein
a. the VH-CDR1 comprises the amino acid sequence set forth in SEQ ID NO: 3;
b. the VH-CDR2 comprises the amino acid sequence set forth in SEQ ID NO: 4;
c. the VH-CDR3 comprises the amino acid sequence set forth in SEQ ID NO: 5;
d. the VL-CDR1 comprises the amino acid sequence set forth in SEQ ID NO: 8;
e. the a VL-CDR2 comprises the amino acid sequence set forth in SEQ ID NO: 9; and
f. the VL-CDR3 comprises the amino acid sequence set forth in SEQ ID NO: 10.

58. An isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein
a. the VH-CDR1 comprises the amino acid sequence set forth in SEQ ID NO: 13;
b. the VH-CDR2 comprises the amino acid sequence set forth in SEQ ID NO: 14;
c. the VH-CDR3 comprises the amino acid sequence set forth in SEQ ID NO: 15;
d. the VL-CDR1 comprises the amino acid sequence set forth in SEQ ID NO: 18;
e. the VL-CDR2 comprises the amino acid sequence set forth in SEQ ID NO: 19; and
f. the a VL-CDR3 comprises the amino acid sequence set forth in SEQ ID NO: 20.

59. An isolated antibody or antigen-binding portion thereof that specifically binds to an integrin-αv heterodimer, wherein the antibody or antigen binding portion thereof comprises a variable heavy region (“VH”) comprising a variable heavy complementarity determining region (VH-CDR) 1, a VH-CDR2, and a VH-CDR3 and a variable light region (“VL”) comprising VL-CDR1, VL-CDR2, and VL-CDR3; wherein
a. the VH-CDR1 comprises the amino acid sequence set forth in SEQ ID NO: 23;
b. the VH-CDR2 comprises the amino acid sequence set forth in SEQ ID NO: 24;
c. the VH-CDR3 comprises the amino acid sequence set forth in SEQ ID NO: 25;
d. the VL-CDR1 comprises the amino acid sequence set forth in SEQ ID NO: 28;
e. the VL-CDR2 comprises the amino acid sequence set forth in SEQ ID NO: 29; and
f. the VL-CDR3 comprises the amino acid sequence set forth in SEQ ID NO: 30.

60. The antibody or antigen-binding portion thereof of any one of claims 51 to 59, wherein the antibody or antigen binding portion thereof comprises a heavy chain variable region (“VH”) and a light chain variable region (“VL”); wherein the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 2, 12, and 22.

61. The antibody or antigen-binding portion thereof of any one of claims 51 to 60, wherein the antibody or antigen binding portion thereof comprises a heavy chain variable region (“VH”) and a light chain variable region (“VL”); wherein the VL comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 17, and 27.

62. The antibody or antigen-binding portion thereof of any one of claims 51 to 61, wherein:
a. the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 2; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7;
b. the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 12; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17; or
c. the VH comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 22; and wherein the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

63. The antibody or antigen-binding portion thereof of any one of claims 51 to 62, wherein:
a. the VH comprises the amino acid sequence set forth in SEQ ID NO: 2, and wherein the VL comprises the amino acid sequence set forth in SEQ ID NO: 7;
b. the VH comprises the amino acid sequence set forth in SEQ ID NO: 12, and wherein the VL comprises the amino acid sequence set forth in SEQ ID NO: 17; or
c. the VH comprises the amino acid sequence set forth in SEQ ID NO: 22, and wherein the VL comprises the amino acid sequence set forth in SEQ ID NO: 27.

64. The antibody or antigen-binding portion thereof of any one of claims 51 to 63, wherein the antibody or antigen binding portion thereof comprises a heavy chain (“HC”) and a light chain (“LC”); wherein the HC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 11, and 21.

65. The antibody or antigen-binding portion thereof of claim 64, wherein the LC comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 6, 16, and 16.

66. The antibody or antigen-binding portion thereof of any one of claims 51 to 65, wherein the antibody or antigen binding portion thereof comprises a heavy chain (“HC”) and a light chain (“LC”); wherein the HC comprises an amino acid sequence selected from SEQ ID NOs: 1, 11, and 21.

67. The antibody or antigen-binding portion thereof of any one of claims 64 to 66, wherein the LC comprises an amino acid sequence selected from SEQ ID NOs: 6, 16, and 26.

68. The antibody or antigen-binding portion thereof of any one of claims 51 to 67, wherein the antibody or antigen binding portion thereof comprises a heavy chain (“HC”) and a light chain (“LC”); wherein:
a. the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 1; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 6;
b. the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16; or
c. the HC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 21; and the LC comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26.

69. The antibody or antigen-binding portion thereof of any one of claims 51 to 68, wherein the antibody or antigen binding portion thereof comprises a heavy chain (“HC”) and a light chain (“LC”); wherein the HC comprises the amino acid sequence set forth in SEQ ID NO: 1; and the LC comprises the amino acid sequence set forth in SEQ ID NO: 6.

70. The antibody or antigen-binding portion thereof of any one of claims 51 to 68, wherein the antibody or antigen binding portion thereof comprises a heavy chain (“HC”) and a light chain (“LC”); wherein the HC comprises the amino acid sequence set forth in SEQ ID NO: 11; and the LC comprises the amino acid sequence set forth in SEQ ID NO: 16.

71. The antibody or antigen-binding portion thereof of any one of claims 51 to 68, wherein the antibody or antigen binding portion thereof comprises a heavy chain (“HC”) and a light chain (“LC”); wherein the HC comprises the amino acid sequence set forth in SEQ ID NO: 21; and the LC comprises the amino acid sequence set forth in SEQ ID NO: 26.

72. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to an amino acid sequence of SEQ ID NO: 2, 12, or 22.

73. The antibody or antigen-binding portion thereof of claim 72, wherein the VL comprises an amino acid sequence having at least about 80%, at least about 85%, at least about 90%, at least about 95%, at least about 96%, at least about 97%, at least about 98%, or at least about 99% sequence identity to an amino acid sequence of SEQ ID NO: 7, 17, or 27.

74. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7.

75. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17.

76. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 22; and the VL comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 27.

77. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 2; and the VL comprises the amino acid sequence set forth in SEQ ID NO: 7.

78. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 12; and the VL comprises the amino acid sequence set forth in SEQ ID NO: 17.

79. An isolated antibody or antigen-binding portion thereof that specifically binds to an integrin-αv heterodimer, wherein the antibody or antigen binding portion thereof comprises a variable heavy region (“VH”) and a variable light region (“VL”); wherein the VH comprises the amino acid sequence set forth in SEQ ID NO: 22; and the VL comprises the amino acid sequence set forth in SEQ ID NO: 27.

80. The antibody or antigen-binding portion thereof of any one of claims 51 to 79, which is an antigen-binding portion.

81. The antigen-binding portion of claim 80, which is a Fab, Fab′, F(ab′)2, single chain Fv (scFv), disulfide linked Fv, IgNar, intrabody, IgGΔCH2, minibody, F(ab′)3, tetrabody, triabody, diabody, single-domain antibody, DVD-Ig, Fcab, mAb2, (scFv)2, or scFv-Fc.

82. A bispecific antibody comprising the antibody or antigen-binding portion thereof of any one of claims 51 to 81, wherein the bispecific antibody is capable of inhibiting integrin-αv-mediated activation of TGFβ.

83. A multispecific antibody comprising the bispecific antibody of any one of claims 1 to 50 and 82 or the antibody or antigen-binding portion thereof of any one of claims 51 to 81, wherein the multispecific antibody is capable of inhibiting integrin-αv-mediated activation of TGFβ.

84. An antibody drug conjugate comprising the bispecific antibody or antigen-binding portion thereof of any one of claims 1 to 50 and 82, the antibody or antigen-binding portion thereof of any one of claims 51 to 81, or the multispecific antibody of claim 83.

85. The bispecific antibody or antigen-binding portion thereof of any one of claims 1 to 50 and 82, the antibody or antigen-binding portion thereof of any one of claims 51 to 81, or the multispecific antibody of claim 83, which is fused to a detectable marker.

86. A polynucleotide or a set of polynucleotides encoding the bispecific antibody of any one of claims 1 to 50 and 82, the antibody or antigen-binding portion thereof of any one of claims 51 to 81, or the multispecific antibody of claim 83.

87. A set of polynucleotides comprising:
a. a first polynucleotide encoding the first heavy chain of any one of claims 5 to 50,
b. a second polynucleotide encoding the second heavy chain of any one of claims 5 to 50, and
c. a third polynucleotide encoding the first light chain of any one of claims 5 to 50.

88. A vector or a set of vectors comprising the polynucleotide of the set of polynucleotides of claim 86 or the set of polynucleotides of claim 87.

89. A cell comprising the bispecific antibody of any one of claims 1 to 50 and 82, the antibody or antigen-binding portion thereof of any one of claims 51 to 81, the multispecific antibody of claim 83, the polynucleotide of the set of polynucleotides of claim 86, or the set of polynucleotides of claim 87.

90. A pharmaceutical composition comprising the bispecific antibody of any one of claims 1 to 50 and 82, the antibody or antigen-binding portion thereof of any one of claims 51 to 81, the multispecific antibody of claim 83, the polynucleotide of the set of polynucleotides of claim 86, or the set of polynucleotides of claim 87, and a pharmaceutically acceptable excipient.

91. A method of making a bispecific antibody or an antigen-binding portion thereof, an antibody or an antigen-binding portion thereof, or a multispecific antibody or an antigen-binding portion thereof, comprising culturing the host cell of claim 90 under suitable conditions and isolating the bispecific antibody.

92. A method of treating a cancer in a subject in need thereof, comprising administering to the subject the bispecific antibody or antigen-binding portion thereof of any one of claims 1 to 50 and 82, the antibody or antigen-binding portion thereof of any one of claims 51 to 81, the multispecific antibody or antigen-binding portion thereof of claim 83, the antibody drug conjugate of claim 84, the polynucleotide of the set of polynucleotides of claim 86, the set of polynucleotides of claim 87, the vector or set of vectors of claim 88, the cell of claim 89, or the pharmaceutical composition of claim 90.

93. The method of claim 92, wherein administration of the bispecific antibody reduces or inhibits metastasis of the cancer in the subject.

94. A method of reducing or inhibiting cancer metastasis in a subject in need thereof, comprising administering to the subject the bispecific antibody or antigen-binding portion thereof of any one of claims 1 to 50 and 87, the antibody or antigen-binding portion thereof of any one of claims 51 to 86, the multispecific antibody or antigen-binding portion thereof of claim 88, the antibody drug conjugate of claim 89, the polynucleotide of the set of polynucleotides of claim 91, the set of polynucleotides of claim 92, the vector or set of vectors of claim 93, the cell of claim 94, or the pharmaceutical composition of claim 95.

95. A method of inhibiting integrin-αvβ1-mediated activation of TGFβ in a subject in need thereof, comprising administering to the subject the bispecific antibody or antigen-binding portion thereof of any one of claims 1 to 50 and 82, the antibody or antigen-binding portion thereof of any one of claims 51 to 81, the multispecific antibody or antigen-binding portion thereof of claim 83, the antibody drug conjugate of claim 84, the polynucleotide of the set of polynucleotides of claim 86, the set of polynucleotides of claim 87, the vector or set of vectors of claim 88, the cell of claim 89, or the pharmaceutical composition of claim 90.

96. The method of claim 95, wherein the subject is afflicted with a cancer.

97. The method of any one of claims 92 to 94 and 96, wherein the cancer comprises a tumor.

98. The method of any one of claims 92 to 93, 96, and 97, wherein the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

99. The method of any one of claims 92 to 98, further comprising administering to the subject an additional anti-cancer therapy.

100. The method of claim 99, wherein the additional anti-cancer therapy comprises a chemotherapy, an immunotherapy, a surgery, a radiotherapy, or any combination thereof.

101. The method of claim 99 or 100, wherein the additional anti-cancer therapy comprises a standard of care therapy.

102. The method of any one of claims 99 to 101, wherein the additional anti-cancer therapy comprises a checkpoint inhibitor.

103. The method of any one of claims 99 to 102, wherein the additional anti-cancer therapy comprises an antibody or an antigen binding portion thereof that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof.

104. The method of claim 103, wherein the anti-PD-1 antibody comprises nivolumab or pembrolizumab.


SEQUENCE LISTING (48 sequences)

SEQ ID NO: 1 - protein, 453 aa, Artificial Sequence
  pSCSTa-hG1 (10392) heavy chain
EVQLVESGGGLVQPGGSLRLSCAASGFNIYYSSIHWVRQAPGKGLEWVASISSSSGSTSTYADSVKGRFTISADTSKNTAYLQMNSLRAEDTAVYYCARSYYGGGYPYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 123 aa, Artificial Sequence
  pSCSTa-hG1 (10392) VH
EVQLVESGGGLVQPGGSLRLSCAASGFDIYYSSIHWVRQAPGKGLEWVASISSSSGSTSTYADSVKGRFTISADTSKNTAYLQMNSLRAEDTAVYYCARSYYGGGYPYALDYWGQGTLVTVSS

SEQ ID NO: 3 - protein, 6 aa, Artificial Sequence
  pSCSTa-hG1 (10392) VH-CDR1
IYYSSI

SEQ ID NO: 4 - protein, 11 aa, Artificial Sequence
  pSCSTa-hG1 (10392) VH-CDR2
SISSSSGSTST

SEQ ID NO: 5 - protein, 11 aa, Artificial Sequence
  pSCSTa-hG1 (10392) VH-CDR3
SYYGGGYPYAL

SEQ ID NO: 6 - protein, 214 aa, Artificial Sequence
  pSCSTa-hK (10392) light chain
DIQMTQSPSSLSASVGDRVTITCRASQSVSSAVAWYQQKPGKAPKLLIYSASSLYSGVPSRFSGSRSGTDFTLTISSLQPEDFATYYCQQYFGGLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Artificial Sequence
  pSCSTa-hK (10392) VL
DIQMTQSPSSLSASVGDRVTITCRASQSVSSAVAWYQQKPGKAPKLLIYSASSLYSGVPSRFSGSRSGTDFTLTISSLQPEDFATYYCQQYFGGLITFGQGTKVEIK

SEQ ID NO: 8 - protein, 5 aa, Artificial Sequence
  pSCSTa-hK (10392) VL-CDR1
SVSSA

SEQ ID NO: 9 - protein, 7 aa, Artificial Sequence
  pSCSTa-hK (10392) VL-CDR2
SASSLYS

SEQ ID NO: 10 - protein, 6 aa, Artificial Sequence
  pSCSTa-hK (10392) VL-CDR3
YFGGLI

SEQ ID NO: 11 - protein, 453 aa, Artificial Sequence
  pSCSTa-hG1 (10404) heavy chain
EVQLVESGGGLVQPGGSLRLSCAASGFNLYSSSIHWVRQAPGKGLEWVAYIYPSYGYTSTYADSVKGRFTISADTSKNTAYLQMNSLRAEDTAVYYCARPAPYYSWYPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 123 aa, Artificial Sequence
  pSCSTa-hG1 (10404) VH
EVQLVESGGGLVQPGGSLRLSCAASGFNLYSSSIHWVRQAPGKGLEWVAYIYPSYGYTSTYADSVKGRFTISADTSKNTAYLQMNSLRAEDTAVYYCARPAPYYSWYPAMDYWGQGTLVTVSS

SEQ ID NO: 13 - protein, 6 aa, Artificial Sequence
  pSCSTa-hG1 (10404) VH-CDR1
LYSSSI

SEQ ID NO: 14 - protein, 11 aa, Artificial Sequence
  pSCSTa-hG1 (10404) VH-CDR2
YIYPSYGYTST

SEQ ID NO: 15 - protein, 11 aa, Artificial Sequence
  pSCSTa-hG1 (10404) VH-CDR3
PAPYYSWYPAM

SEQ ID NO: 16 - protein, 214 aa, Artificial Sequence
  pSCSTa-hK (10404) light chain
DIQMTQSPSSLSASVGDRVTITCRASQSVSSAVAWYQQKPGKAPKLLIYSASSLYSGVPSRFSGSRSGTDFTLTISSLQPEDFATYYCQQASGSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 107 aa, Artificial Sequence
  pSCSTa-hK (10404) VL
DIQMTQSPSSLSASVGDRVTITCRASQSVSSAVAWYQQKPGKAPKLLIYSASSLYSGVPSRFSGSRSGTDFTLTISSLQPEDFATYYCQQASGSPFTFGQGTKVEIK

SEQ ID NO: 18 - protein, 5 aa, Artificial Sequence
  pSCSTa-hK (10404) VL-CDR1
SVSSA

SEQ ID NO: 19 - protein, 7 aa, Artificial Sequence
  pSCSTa-hK (10404) VL-CDR2
SASSLYS

SEQ ID NO: 20 - protein, 6 aa, Artificial Sequence
  pSCSTa-hK (10404) VL-CDR3
ASGSPF

SEQ ID NO: 21 - protein, 452 aa, Artificial Sequence
  pSCSTa-hG1 (11867) heavy chain
EVQLVESGGGLVQPGGSLRLSCAASGFDFDSYAIHWVRQAPGKGLEWVAYFYPGHDYSTYADSVKGRFTISADTSKNTAYLQMNSLRAEDTAVYYCARPSPYFSWHQAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 122 aa, Artificial Sequence
  pSCSTa-hG1 (11867) VH
EVQLVESGGGLVQPGGSLRLSCAASGFDFDSYAIHWVRQAPGKGLEWVAYFYPGHDYSTYADSVKGRFTISADTSKNTAYLQMNSLRAEDTAVYYCARPSPYFSWHQAMDYWGQGTLVTVSS

SEQ ID NO: 23 - protein, 6 aa, Artificial Sequence
  pSCSTa-hG1 (11867) VH-CDR1
FDSYAI

SEQ ID NO: 24 - protein, 10 aa, Artificial Sequence
  pSCSTa-hG1 (11867) VH-CDR2
YFYPGHDYST

SEQ ID NO: 25 - protein, 11 aa, Artificial Sequence
  pSCSTa-hG1 (11867) VH-CDR3
PSPYFSWHQAM

SEQ ID NO: 26 - protein, 214 aa, Artificial Sequence
  pSCSTa-hK (11867) light chain
DIQMTQSPSSLSASVGDRVTITCRASQSVSSAVAWYQQKPGKAPKLLIYSASSLYSGVPSRFSGSRSGTDFTLTISSLQPEDFATYYCQQATGSEVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 69 aa, Artificial Sequence
  pSCSTa-hK (11867) VL
DIQMTQSPSSLSASVGDRVTITCRASQSVSSAVAWYQQKPGKAPKLLIYSASSLYSGVPSRFSGSRSGT

SEQ ID NO: 28 - protein, 5 aa, Artificial Sequence
  pSCSTa-hK (11867) VL-CDR1
SVSSA

SEQ ID NO: 29 - protein, 7 aa, Artificial Sequence
  pSCSTa-hK (11867) VL-CDR2
SASSLYS

SEQ ID NO: 30 - protein, 6 aa, Artificial Sequence
  pSCSTa-hK (11867) VL-CDR3
ATGSEV

SEQ ID NO: 31 - protein, 452 aa, Artificial Sequence
  pSCSTa-Fck79 (10392) knob heavy chain
EVQLVESGGGLVQPGGSLRLSCAASGFNIYYSSIHWVRQAPGKGLEWVASISSSSGSTSYADSVKGRFTISADTSKNTAYLQMNSLRAEDTAVYYCARSYYGGGYPYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 122 aa, Artificial Sequence
  pSCSTa-Fck79 (10392) knob VH
EVQLVESGGGLVQPGGSLRLSCAASGFNIYYSSIHWVRQAPGKGLEWVASISSSSGSTSYADSVKGRFTISADTSKNTAYLQMNSLRAEDTAVYYCARSYYGGGYPYALDYWGQGTLVTVSS

SEQ ID NO: 33 - protein, 330 aa, Artificial Sequence
  pSCSTa-Fck79 (10392) knob Constant Heavy
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 453 aa, Artificial Sequence
  pSCSTa Fch8c (10404) hole heavy chain
EVQLVESGGGLVQPGGSLRLSCAASGFNLYSSSIHWVRQAPGKGLEWVAYIYPSYGYTSTYADSVKGRFTISADTSKNTAYLQMNSLRAEDTAVYYCARPAPYYSWYPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 123 aa, Artificial Sequence; identical to SEQ ID NO: 12
  pSCSTa Fch8c (10404) hole VH

SEQ ID NO: 36 - protein, 330 aa, Artificial Sequence
  pSCSTa Fch8c (10404) hole Constant Heavy
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 452 aa, Artificial Sequence
  pSCSTa Fch8c (11867) hole heavy chain
EVQLVESGGGLVQPGGSLRLSCAASGFDFDSYAIHWVRQAPGKGLEWVAYFYPGHDYSTYADSVKGRFTISADTSKNTAYLQMNSLRAEDTAVYYCARPSPYFSWHQAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 122 aa, Artificial Sequence; identical to SEQ ID NO: 22
  pSCSTa Fch8c (11867) hole VH

SEQ ID NO: 39 - protein, 330 aa, Artificial Sequence; identical to SEQ ID NO: 36
  pSCSTa Fch8c (11867) hole Constant Heavy

SEQ ID NO: 40 - protein, 330 aa, Artificial Sequence [sequence omitted]

SEQ ID NO: 41 - protein, 330 aa, Artificial Sequence
  alotypic variant IgG1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, Artificial Sequence
  IgG1.1f effectorless human IgG1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, Artificial Sequence
  IgG1.3f effectorless human IgG1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 327 aa, Artificial Sequence
  wild-type human IgG4 constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 107 aa, Artificial Sequence [sequence omitted]

SEQ ID NO: 46 - protein, 5 aa, Artificial Sequence
  heavy chain C-terminus peptide
LSPGK

SEQ ID NO: 47 - protein, 4 aa, Artificial Sequence
  heavy chain C-terminus peptide
LSPG

SEQ ID NO: 48 - protein, 3 aa, Artificial Sequence
  heavy chain C-terminus peptide
LSP